(19) United States
(12) Patent Application Publication
(10) Pub. No.: US 2005/0289669 A1
(43) Pub. Date: Dec. 29, 2005

(54) TTG3 deficient plants, nucleic acids, polypeptides and methods of use thereof

(76) Inventors: Jiangxin Wan, Kingston (CA); Yafan Huang, Kingston (CA); Angela Patricia Gilley Sample, Inverary (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/070,992

(22) Filed: Mar. 3, 2005

Related U.S. Application Data
(60) Provisional application No. 60/549,655, filed on Mar. 3, 2004.

(51) Int. Cl. A01H 1/00; C12N 15/82; C07H 21/04; C12N 5/04

(52) U.S. Cl. 800/282; 435/419; 435/468; 536/23.2

(57) ABSTRACT

The present invention provides novel isolated polynucleotides and polypeptides encoded by the TTG3 polynucleotides. The invention additionally provides methods of constructing transgenic plants that have altered levels of TTG3 polynucleotides and polypeptides.

### RELATED APPLICATIONS

This application claims priority to U.S. Ser. No. 60/549,655 filed Mar. 3, 2004 and which is incorporated herein by reference in its entirety.

### FIELD OF THE INVENTION

The invention relates to novel plant transparent testa glabra polynucleotides, polypeptides and promoter. Also included are transgenic plants expressing the novel polynucleotides and polypeptides. Also included are transgenic plant cells, tissues and plants having novel phenotypes resulting from the expression of these polynucleotides in either the sense or antisense orientation. Also included are plant cells, tissues and plants having novel phenotypes resulting from a non-naturally occurring mutation of these polynucleotides.

### BACKGROUND OF THE INVENTION

A goal of plant biology is the improvement of agronomically important traits in plants. This includes both improvements to existing crop species, as well as modification of species such that they become viable crop commodities. Many traits have additional applications outside agriculture in such fields as horticulture and landscaping plant species. Among the traits that are desirably altered include the fatty acid composition of seed oil and quantity thereof, seed or seed coat composition, including protein, oil or fiber qualities and quantities. Further traits include pest resistance, environmental and nutritional stress resistance, herbicide tolerance and modification of various phenotypic traits such as flower color or morphology, flowering pattern, leaf structure or stem structure. Plants that are altered in these or other traits have scientific, commercial or social benefit.

The production and identification of transgenic plants is a vital component of present day plant genetic and biochemical research. Selection of a plant cell, tissue, seed or plant which has undergone a transformation event from one that has not is a critical step and one that can be arduous. To facilitate this selection, a selectable marker is often introduced that provides a novel trait to the transgenic plant cell, tissue, seed or plant. Commonly, this selectable marker confers a trait that can be used to identify the plant cell, tissue, seed or plant that is transgenic; for example, herbicide resistance, antibiotic resistance or growth on restrictive carbon sources or metabolites. These methods are referred to as positive selection mechanisms and are generally preferred over negative selection mechanisms. Negative selection provides an environment in which a non-transformed plant cell, tissue, seed or plant thrives while the transgenic plant cell, tissue, seed or plant is restricted in its development. Transformation being a rare event, large numbers of plant cells, tissues, seeds or plants must be screened to identify those that are potentially transgenic.

The selectable marker is often not the gene of interest, which can be a second heterologous gene sequence transformed at the same time, often on the same construct, to generate the transgenic plant. The transgenic plants identified through the selectable marker screen are then often subjected to molecular analysis to confirm the presence of the transgene(s) and optionally, to determine gene expression levels. Molecular analysis is performed on T2 plants, or in some cases, later generations are required for analysis due to zygosity considerations of the genes under scrutiny. In some cases, molecular analysis is performed after the zygosity of the transgenic material is assessed, often in the T3 generation.

Preservation of a selectable marker or gene encoding such a marker in subsequent generations can be undesirable. An efficiency drag or fitness limitation may result from the presence of the selectable marker or, from a public acceptability perspective; it may be undesirable to maintain the selectable marker in the genetic background. A means of selecting transformed plants at an early stage on a visual basis without requiring a non-plant gene insertion would be clearly advantageous.

Further requirements of genetic engineering are the components required for gene expression; namely, a promoter, an expressed gene sequence and, optionally, a transcription terminator. Useful promoters are often a key to the success of transgenic strategies and the availability of characterized promoters is of significant importance.

### SUMMARY OF THE INVENTION

The present invention is based in part upon the discovery of novel transparent testa glabrous (TTG3) nucleic acid sequences, promoters and polypeptides isolated from Arabidopsis thaliana The nucleic acids, polynucleotides, proteins and polypeptides, or fragments thereof described herein are collectively referred to as TTG3 nucleic acids and polypeptides.

Accordingly, in one aspect, the invention provides an isolated nucleic acid molecule that includes the sequence of SEQ ID NO:1, SEQ ID NO:2, SEQ ID NO:6 or SEQ ID NO:7 or fragment, homolog, analog or derivative thereof. The nucleic acid can be, e.g., a genomic DNA fragment, a cDNA molecule or an RNA molecule. Preferably, the nucleic acid is naturally occurring. The invention also provides a nucleic acid sequence that is complementary to the nucleic acid sequence of SEQ ID NO:1, SEQ ID NO:2, SEQ ID NO:6 or SEQ ID NO:7 or fragment thereof. Also included in the invention is a vector containing one or more of the nucleic acids described herein, and a cell containing the vectors or nucleic acids described herein. The fragment contains 20, 50, 100, 120, 150, 200, 300, 400, 500, 600, 700, 750, 800, 900, or more consecutive nucleotides of SEQ ID NO:1, SEQ ID NO:2, SEQ ID NO:6 or SEQ ID NO:7 (i.e., sense orientation) Optionally the fragment contains 20, 50, 100, 120, 150, 200, 300, 400, 500, 600, 700, 750, 800, 900, or more consecutive nucleotides that are complementary to SEQ ID NO:1, SEQ ID NO:2, SEQ ID NO:6 or SEQ ID NO:7 (i.e., anti-sense orientation). If the nucleic acid fragment is in the sense orientation it is capable of rescuing the ttg3 phenotype. By rescuing the mutant ttg3 phenotype is meant that when the nucleic acid fragment is used to transform a plant cell from a plant having the mutant ttg3 phenotype the progeny of the transformed cell has a normal phenotype, i.e., wild-type phenotype. Alternatively, if the nucleic acid fragment is in the anti-sense orientation it is capable of inducing the ttg3 phenotype. By inducing the mutant ttg3 phenotype is meant that when the nucleic acid fragment is used to transform a plant cell from a plant having normal, e.g., wild-type phenotype the progeny of the transformed cell has mutant ttg3 phenotype.

In another aspect, the invention provides an isolated TTG3 promoter nucleic acid molecule that includes the sequence of SEQ ID NO:5 or SEQ ID NO:8 or a fragment, homolog, analog or derivative thereof. The TTG3 promoter nucleic acid is less than 1215 nucleotides in length. Preferably, the TTG3 promoter is less than 800, 750, 600, 400, or 200 nucleotides in length. Accordingly, in another aspect, the invention provides nucleic acid constructs including a TTG3 promoter (e.g., SEQ ID NO:5 or SEQ ID NO:8) fragment, homolog, analog or derivative thereof operably linked to a nucleotide sequence encoding a gene. Alternatively, the TTG3 promoter is operably linked to a non-translatable mRNA molecule of a gene. A non-translatable mRNA molecule includes, for example, an antisense nucleic acid, a hairpin RNA or a microRNA.

In a further aspect, the invention includes host cells transformed with a vector comprising any of the nucleic acid molecules described above. The invention is also directed to plants and cells transformed with a TTG3 nucleic acid or a vector comprising a TTG3 nucleic acid.

In a further aspect, the invention includes a substantially purified TTG3 polypeptide, e.g., any of the TTG3 polypeptides encoded by an TTG3 nucleic acid, and fragments, homologs, analogs, and derivatives thereof. Accordingly, in one aspect, the invention provides an isolated polypeptide molecule that includes the sequence of SEQ ID NO:4.

In another aspect, the invention provides a plant having altered, e.g., increased or decreased expression or activity of the TTG3 gene, e.g., SEQ ID NO:1, SEQ ID NO:2, SEQ ID NO:6 or SEQ ID NO:7. Altered TTG3 expression or activity is a result of for example a non-naturally occurring mutation in TTG3 gene. When TTG3 expression or activity is decreased the plant has a phenotype including, but not limited to, yellow seed color, transparent testa, reduced anthocyanin production, reduced proanthocyaninidin production, reduced flavanol production, reduced trichome presence, increased oil biosynthesis, oil quantity or oil quality, and decreased fiber composition compared to a wild-type plant. Alternatively, when the TTG3 expression or activity is increased, the plant has a phenotype including, but not limited to, decreased oil biosynthesis, oil quantity or oil quality, and increased fiber composition compared to a wild-type plant. Mutation in a TTG3 gene is achieved by a variety of means known in the art such as, but not limited to, chemical or fast-neutron irradiation mutagenesis or insertional inactivation. Additionally, modifications in a regulatory element, e.g., SEQ ID NO:5 or SEQ ID NO:8, of TTG3 gene can be used to alter the expression of the gene. Such a TTG3 regulatory sequence is shown as SEQ ID NO:5 or SEQ ID NO:8.

The invention also includes a method of producing a transgenic plant. The plant has increased or decreased TTG3 expression or activity or an altered phenotype such as altered seed oil content, altered seed fiber content, altered trichome production, altered trichome structure, transparent testa, altered anthocyanin production, altered proanthocyaninidin production and altered flavanol production compared to a wild type plant. Transgenic plants are produced by introducing into one or more cells of a plant a compound that alters (e.g., increases or decreases) TTG3 expression or activity in the plant. In one aspect the compound is a TTG3 nucleic acid or polypeptide. Alternatively, the compound is a TTG3 double stranded RNA-inhibition hair-pin nucleic acid or TTG3 antisense nucleic acid. For example, when the compound decreases the production of a TTG3 gene, or gene product, the resulting plant has a phenotype such as increased seed oil content, decreased seed fiber content, reduced trichome production, altered trichome structure, transparent testa, reduced anthocyanin production reduced proanthocyaninidin production and reduced flavanol production compared to a wild type plant. In contrast, when the compound increases the production of a TTG3 gene, or gene product, the resulting plant has a phenotype such as decreased seed oil content, or increased seed fiber content compared to a wild type plant. A wild-type plant is a plant that does not have altered expression of TTG3 expression or activity. For example, a wild-type plant is a plant that has not been contacted with a compound that alters TTG3 expression or activity or does not have a naturally occurring mutation in the TTG3 gene

Transformed plants or plants cells are identified and selected by the reversion of a ttg3 phenotype to a wild-type phenotype through the introduction of a gene construct comprising a TTG3 nucleic acid sequence, e.g., SEQ ID NO:1, SEQ ID NO:2, SEQ ID NO:6 or SEQ ID NO:7 or fragment thereof. The presence of the introduced TTG3 nucleic acid sequence restores the wild-type gene function and loss of at least a distinguishing feature of the mutant plant cell, or seed. Alternatively, transformed plants or plants cells are identified and selected by introducing a gene construct that inhibits or eliminates TTG3 gene expression or function thereby resulting in the appearance of at least a ttg3 mutant phenotype. For example, an antisense copy of SEQ ID NO:1, SEQ ID NO:2, SEQ ID NO:6 or SEQ ID NO:7 or a portion thereof, is introduced into a wild-type background resulting in at least a distinguishing feature of the ttg3 mutant plant cell, seed or plant thereby indicating a transformation event. Optionally, a second nucleic acid sequence operably associated with a promoter is included within said first construct having a TTG3 gene or anti-sense TTG3 gene of fragment thereof. The second nucleic acid encodes a heterologous gene of interest.

Also included in the invention is the seed, and progeny of the transformed plants or cells produced by the methods of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF INVENTION

The present invention provides a novel transparent testa glabra (TTG) nucleic acid sequences (SEQ ID NO:1, SEQ ID NO:2, SEQ ID NO:6 and SEQ ID NO:7), encoded polypeptide (SEQ ID NO:4,) and fragments and mutants thereof isolated from Arabidopsis thaliana (At). More specifically, the invention provides a gene and a gene product associated with morphological and biochemical features such as trichome morphology, pigment production and seed characteristics including color, fiber and oil composition. Also provide by the invention are methods of manipulating the gene in plants to down-regulate or up-regulate the endogenous gene expression or function resulting in a plant with an altered phenotype. Also include is gene promoter sequence useful in directing transcription of an operably linked downstream nucleic acid sequence. The wild-typoe sequences are collectively referred to herein as “TTG3 nucleic acids”, TTG3 polynucleotides”, “TTG3 antisense nucleic acids” or TTG3 promoter sequence” and the corresponding encoded polypeptide is referred to as a “TTG3 polypeptide” or “TTG3 protein”. The mutant sequences are are collectively referred to herein as “ttg3 nucleic acids”, ttg3 polynucleotides”, “ttg3 antisense nucleic acids” or ttg3 promoter sequence” and the corresponding encoded polypeptide is referred to as a “ttg3 polypeptide” or “ttg3” protein”. Unless indicated otherwise, “TTG, TTG3,TTG mutant or TTG3 mutant” is meant to refer to any of the novel sequences disclosed herein.

Table A below summarizes the nucleic acids and polypeptides according to the invention.

TABLE 1

| | | |
|---|---|---|
| SEQ | | |
| ID | | |
| NO: | Description | Sequence |

| | | |
|---|---|---|
| 1 | Genomic ttg DNA | |
| 2 | SEQ ID NO:1 + | |
| | Newtranscribed Seq. | |
| 3 | cDNA (AC018664) | |
| 4 | predicted polypeptide | |
| | (AC018664) | |
| 5 | Predicted promoter A | |
| 6 | TTG3 rescue seq | |
| 7 | transcribed Seq. B (EST) | |
| 8 | predicted promoter B | |
| 9 | HPR promoter | |
| 10 | BAN promoter | |
| 11 | SC promoter | |
| 12 | Napin promoter | |
| 13 | Promoter P-TTG3 | |
| 14 | Promoter P700 | |
| 15 | Promoter P400 | |
| 16 | Promoter P200 | |
| 17 | Promoter P-TDNA | |
| 18 | Adapter | CTAATACGACTCACTATAGGGCTCGAGCGGCCGCCCGGGCAGGT |
| 19 | Adapter | ACCTGCCC-NH₂ |
| 20 | Primer AP1 | GGATCCTAATACGACTCACTATAGGGC |
| 21 | Primer 28walk1 | AGCTGGCGTAATAGCGAAGA |
| 22 | Primer AP2 | CTATAGGGCTCGAGCGGC |
| 23 | Primer 28walk2 | CGTTGGAGTCCACGTTCTTT |
| 24 | Primer TTG-XbaFW | AAATCTAGAATGATGGTGAGTGGCCTATATTC |
| 25 | Primer TTG-SacRV | AAAGAGCTCTTAGCATCGAATACACATGTAACCTGAGGAGA |
| 26 | Primer PTTG-XbaFW | AAATCTAGAGCGAATACGTAAACGTATGACCTGGTTTTGTTC |
| 27 | TTG1-FW | AAAGAGCTCATGGATAATTCAGCTCCAGA |
| 28 | TTG1-RV | AAAGAGCTCACTCTAAGGAGCTGCATTTTGTT |
| 29 | Primer b-tubulin-FW | GAGATTCTTCACATCCAGGG |
| 30 | Primer b-tubulin-RV | CATCTCGTCCATTCCCTCAC |
| 31 | Primer TTG-SmaRV | AAACCCGGGTACAAGTCCACTGTCCACCACACATCAAAG |
| 32 | Primer TTG-BamFW | AAAGGATCCATGATGGTGAGTGGCCTATATTC |
| 33 | Primer TTG-XbaRV | AAATCTAGATTAGCATCGAATACACATGTAACCTGAGGAGA |
| 34 | Primer TTG-B-XbaFW | AAATCTAGAGATTCTTAAAGACAAAATTAAAAT GACTCTG |
| 35 | Primer TTG-B-BamRV | AAAGGATCCCACAAGTGACGATTTTATTAAGATGAACAT |
| 36 | Primer 35S FW | GCCGACAGTGGTCCCAAAGATGG |
| 37 | Primer Nosterm | GCAAGACCGGCAACAGGA |
| 38 | Primer HP-TTG3-2BamFW | AAAGGATCCACAGCAGTACATGGTCGGCCCTTGT |
| 39 | Primer HP-TTG3-2XbaRV | AAATCTAGATGAACATAACATACAACGAACGAGCA |
| 40 | Primer HP-TTG3-2SacFW | AAAGAGCTCACAGCAGTACATGGTCGGCCCTTGT |
| 41 | Primer HP-TTG3-2SacRV | AAAGAGCTCTGAACATAACATACAACGAACGAGCA |
| 42 | Primer HP-TTG3-3BamFW | AAAGGATCCGACCGTTGATTGTCTTTAGATGATCAGA |
| 43 | Primer HP-TTG3-3XbaRV | AAATCTAGACCATCTATCATCCTTCGCCGTGAAGCCA |
| 44 | Primer HP-TTG3-3SacFW | AAAGAGCTCGACCGTTGATTGTCTTTAGATGATCAGA |
| 45 | Primer HP-TTG3-3SacRV | AAAGAGCTCCCATCTATCATCCTTCGCCGTGAAGCCA |
| 46 | Primer TTG3-SacFW | AAAGAGCTCATGATGGTGAGTGGCCTATATTC |
| 47 | Primer BAN-HindFW | AAAAAGCTTATTTGCTTAAGGCCAGATTCT |
| 48 | Primer BAN-XbaRV | AAATCTAGACTTGATGAGACTTGATGAGAATAG |
| 49 | Primer ScPr-XbaRV: | AAATCTAGACTTTTGATTGTTTTTATCTTTTGG |
| 50 | Primer ScPr-HindFW | AAAAAGCTTAAATTGACATTATATATGAAAGACAA |
| 51 | Primer NAP-A-HIND-FW | AAAAGCTTTTAAACCAACTTAGTAAACGTTTTTTT |
| 52 | Primer NAP-A-XBA-RF | AAATCTAGACGTGTATGTTTTTAATCTACATTGTATTGA |
| 53 | HPRP1Hind | AAAAAGCTTGAAGCAGCAGAAGCCTTGAT |
| 54 | HPRP2Bam | AAAGGATCCCGCCATGGTAGAGAAAAGAGA |
| 55 | Primer HPR1 | GAAGCAGCAGAAGCCTTGAT |
| 56 | Primer TTG3 Pro-700FW | AAATCTAGATATGGCTTCATCTCTTAAGAAATACTTTCCA |
| 57 | Primer TTG3 Pro-400FW | AAATCTAGACATCCTATGAAATTTAACTCATAAAAGTGTCA |
| 58 | Primer TTG3 Pro-200FW | AAATCTAGAATTCTAAGGTCTATAAACATATTGGATGCA |
| 59 | Primer TTG3 Pro-TDNAFW | AAATCTAGAAATTCTTTACTTACCAATCCGGTGGAGAC |
| 60 | Primer TTG3 Pro-SmaRV | AATCCCGGGAGAGAGAAGAGAGAGAGGGCCAGAGTCAT |
| 61 | T5-Sma-FW | ATTCCCGGGATTCTTAAAGACAAAATTAAAATGACTCTG |
| 62 | T3-Bam-RV | AAAGGATCCCACAAGTGACGATTTTATTAAGATGAACAT |
| 63 | T5-200Sma-FW | ATTCCCGGGTCTAGCATCATCGGCTCTTTCAG |
| 64 | T5-400Sma-FW | ATTCCCGGGTATCCTTTGAGTTTAAGGTTTACGGAT |
| 65 | T5-600Sma-FW | ATTCCCGGGTCCTTACTTCTGTTTTGTAGTTGTTGGA |
| 66 | T5-800Sma-FW | ATTCCCGGGTCTCTAAATGTAATGTGGGCCAGTT |
| 67 | T3-200Bam-RV | AAAGGATCCATCTATCATCCTTCGCCGTGA |
| 68 | T3-400Bam-RV | AAAGGATCCAGCTTGAATATAAGAGATACAGCTAAAG |
| 69 | T3-600Bam-RV | AAAGGATCCTCTAAAGACAATCAACGGTCGAAACGT |
| 70 | T3-800Bam-RV | AAAGGATCCTCAACCATCACCGACGACATCAATTTA |
| 71 | pGAD-TTG3 FL | Full length expression construct 915 bp |
| 72 | pGAD-T5-D200 | 5′ deletion construct 718 bp |
| 73 | pGAD-T5-D400 | 5′ deletion construct 517 bp |
| 74 | pGAD-T5-D600 | 5′ deletion construct 319 bp |
| 75 | pGAD-T5-D800 | 5′ deletion construct 120 bp |
| 76 | pGAD-T3-D200 | 3′ deletion construct 717 bp |
| 77 | pGAD-T5-D400 | 3′ deletion construct 520 bp |
| 78 | pGAD-T5-D600 | 3′ deletion construct 319 bp |
| 79 | pGAD-T5-D800 | 3′ deletion construct 123 bp |
| 80 | Complement | |
| | of SEQ ID NO:2 | |

The TTG3 nucleic acid was identified in a population of plants carrying a T-DNA insert. The plants were observed to lack trichomes on the leaves and stems, although a few were visible on the leaf margins, albeit with altered morphology. At maturation the plant was observed to produce seeds having a yellow seed color rather than the darker color present in wild-type Arabidopsis. Based upon the nature of the displayed phenotypes (e.g., transparent testa phenotype and a glabrous phenotype) the affected gene is related to TTG1 and TTG2 and was named TTG3. Molecular analysis identified the gene sequence (i.e., SEQ ID NO:1, SEQ ID NO:2, SEQ ID NO:6 or SEQ ID NO:7) and the location of the T-DNA indicated that the endogenous gene had been down-regulated by disruption of normal promoter function.

Trichomes (leaf hairs) are specialized epidermal cells having three radial projecting branches and are believed to provide physical protection from predator attack ion many plant species. In wild-type Arabidopsis plants, trichomes are distributed evenly over the leaf surfaces and can also be found on the stem, cauline leaves and sepals. The epidermis of the hypocotyle, cotyledon, petal, stamen and carpel normally do not possess trichome structures. Flavonoid compounds are a class of secondary metabolites which include the anthocyanins, proanthocyanidins and flavonols. These subclasses of flavonoid compounds contribute to plant pigmentation in that anthocyanins contribute the red, purple and blue pigments in vegetative tissues; proanthocyanidins (condensed tannins) give the brown pigment to seed coat and flavonols give flowers, seeds and vegetative tissues a colorless or yellowish hue.

Arabidopsis is a model plant system in plant molecular genetics and substantial work has produced and characterized numerous mutants and generated the nucleic acid sequence of the Arabidopsis genome. Mutants of trichome development and anthocyanin production have been identified which exert control over either one or both phenotypes (Koornneef, 1981). Mutants affecting tannin synthesis in the seed coat resulting in pale or yellow seed color are termed transparent testa (tt). Mutants affecting trichome production or structure are termed glabra (gl). Mutants that exert influence on both trichome production or structure and flavonoid biosynthesis resulting in the phenotypes of both tt and gl mutants are termed transparent testa glabra (ttg). To date, 25 independent loci have been identified by mutational approaches (TT1 to TT19, GL1 to GL3, and TTG1 and TTG2) (Koornneef et al., 1982; Koornneef, 1990; Shirley et al., 1992; 1995; Focks et al.,1999; Debeaujon et al., 2000; Nesi et al., 2002; Winkel-Shirley, 2002; Shikazono et al., 2003; Kitamura et al., 2004; Herman and Marks, 1989; Ramsay et al., 2003; Bernhardt et al., 2003; Walker et al., 1999; Johnson et al., 2002). In addition, a locus named BANYULS (BAN) has been shown to be involved in the tannin biosynthetic pathway (Devic et al., 1999; and Debeaujon et al., 2003).

A “ttg3 phenotype” refers to a plant having reduced expression of a TTG3 gene and displaying the phenotypic characteristics including transparent testa, reduced or eliminated anthocyanin synthesis, reduced proanthocyaninidin production, reduced flavanol production, reduced trichome presence, altered oil biosynthesis (i.e. decreased oil), oil quantity or oil quality, or altered fiber composition (i,e., increased fiber). The seed testae of many seeds are colored as a result of proanthocyaninidin accumulation in this cell layer. In ttg3 the lack of the proanthocyaninidins (tannins) accumulation results in a transparent cell layer thereby allowing the visualization of underlying cells, the cotyledons in dicot species, which generally give the seed a yellow color.

A “TTG3 phenotype” refers to a plant having normal expression of a TTG3 gene and displaying the characteristics of a wild-type plant.

“TTG3” refers to a wild-type plant phenotype or wild-type gene sequence as contextually appropriate.

“ttg3” refers to a mutant plant deficient in TTG3 expression and displaying a ttg3 phenotype or mutant gene sequence as contextually appropriate.

The terms oil and lipid may be used herein as interchangeable terms unless specifically stated to the contrary.

TTG1 encodes a WD40 repeat protein that may not directly act as a transcription factor, but rather bind to other proteins to promote the initiation of trichome and flavonoid biosynthesis. Except for a few unbranched trichomes which may arise at the leaf margins, the ttg1 mutant lacks trichomes on leaf and stem surfaces and does not accumulate anthocyanins in vegetative tissue or tannins in the seed coat.

TTG2 encodes a zinc finger-like WRKY transcription factor that regulates trichome development and production of tannins and mucilage in seed coat. ttg2 mutant plants have a reduced number of trichomes that are structurally unbranched and pale seed coats, albeit not as pale as seen in ttg1 mutants. TTG2 has reduced mucilage production in the seed coat, while the level of anthocyanidins is normal. Characterization of the ttg1ttg2 double mutant suggests that while TTG1 function apparently precedes TTG2 in trichome and seed coat tannin production, TTG2 does not share functions with TTG1 in anthocyanidin and root hair development. Although the forgoing has been demonstrated in the dicot species, Arabidopsis, monocots may share a similar mechanism for trichome development and flavonoid production. For example, the maize R gene is a specific transcriptional activator required for anthocyanidin production in maize that, when expressed in a ttg1 Arabidopsis mutant, resulted in the restoration of anthocyanidin and trichome production, thereby indicating that TTG1 and R may be functional homologues or that TTG1 activates a R homologue (Lioyd, et al., 1992). Additionally, a required gene for anthocyanidin production, PAC1, encoding a WD40 motif, closely resembles the Arabidopsis TTG1 gene; although, the pac1 mutant does not possess all the ttg1 phenotypes observed in Arabidopsis (Carey et al., 2004). Over-expression of PAC1 in an Arabidopsis ttg1 background results in phenotypic rescue of the ttg1 phenotypes.

Based on their structural and functional relatedness to known TTG nucleic acids, the TTG3 nucleic acids are novel members of the TTG family of nucleic acids and proteins. TTG3 nucleic acids, and their encoded polypeptides, according to the invention are useful in a variety of applications and contexts. For example, the nucleic acids (i.e., sense or antisense TTG3 nucleic acids) are used produce transgenic plants that have an altered seed oil content, altered seed fiber content, reduced trichome production, altered trichome structure, transparent testa, reduced anthocyanin production reduced proanthocyaninidin production and reduced flavanol production compared to a wild type plant.

This invention includes methods to up-regulate the TTG3 activity in transgenic plants, cells and tissue cultures by using an over-expression vector construct and methods to down-regulate the TTG3 activity in transgenic plants, cells and tissue cultures by using a double stranded RNA-inhibition, hairpin vector constructs or antisense constructs. Alteration (i.e., upregulation or downregulation) of TTG3 activity or expression results in transgenic plants with altered phenotypes as described below. These methods are by way of example to produce the up-regulation or down-regulation effects and are not meant to be limiting as to the method of achieving this outcome.

Additionally the nucleic acids and polypeptides according to the invention are used for determining the successful transformation of a plant. Optionally, the plant is a further tramsformed with a heterologous gene.

The nucleic acids and polypeptides according to the invention may be used as targets for the identification of small molecules that modulate or inhibit, TTG3 activity. Alternatively, the TTG3 nucleic acids and polypeptides can be used to identify proteins that are members of the TTG family of nucleic acids and proteins.

Additional utilities for TTG3 nucleic acids and polypeptides according to the invention are disclosed herein.

TTG3 Nucleic Acids

The nucleic acids of the invention include those that encode a TTG3 gene, a transcribed TTG3 RNA or TTG3 polypeptide or protein. As used herein, the terms polypeptide and protein are interchangeable. A TTG3 RNA is translated. Alternatively, the TTG3 RNA is non-translated.

In some embodiments, a TTG3 nucleic acid encodes a mature TTG3 polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation, farnesylation, geranylgeranylation or phosphorylation. In general, a mature polypeptide or protein may result from the operation of only one of these processes, or a combination of any of them.

Among the TTG3 nucleic acids is the nucleic acid whose sequence is provided in SEQ ID NO: 1, SEQ ID NO:2, SEQ ID NO:6 or SEQ ID NO:7 or a fragment thereof. Additionally, the invention includes mutant or variant nucleic acids of SEQ ID NO: 1, SEQ ID NO:2, SEQ ID NO:6 or SEQ ID NO:7 or a fragment thereof, any of whose bases may be changed from the corresponding base shown in SEQ ID NO: 1, SEQ ID NO:2, SEQ ID NO:6 or SEQ ID NO:7, while still encoding a RNA molecule or a protein that maintains at least one of its TTG3-like activities and physiological functions. The invention further includes the complement of the nucleic acid sequence of SEQ ID NO: 1, SEQ ID NO:2, SEQ ID NO:6 or SEQ ID NO:7, including fragments, derivatives, analogs and homologs thereof. Complement nucleic acid TTG3 sequences include SEQ ID NO: 80. The invention additionally includes nucleic acids or nucleic acid fragments, or complements thereto, whose structures include chemical modifications.

One aspect of the invention pertains to isolated nucleic acid molecules that encode TTG3 proteins or biologically active portions thereof. Also included are nucleic acid fragments sufficient for use as hybridization probes to identify TTG3-encoding nucleic acids (e.g., TTG3 mRNA) and fragments for use as polymerase chain reaction (PCR) primers for the amplification or mutation of TTG3 nucleic acid molecules. As used herein, the term “nucleic acid molecule” is intended to include DNA molecules (e.g., cDNA or genomic DNA), RNA molecules (e.g, mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TTG3 nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, SEQ ID NO:2, SEQ ID NO:6 or SEQ ID NO:7 or a complement of any of this nucleotide sequence, can be isolated using standard molecular biology techniques and the sequence information provided herein. Using all or a portion of the nucleic acid sequence of SEQ ID NO: 1, SEQ ID NO:2, SEQ ID NO:6 or SEQ ID NO:7 as a hybridization probe, TTG3 nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, NY, 1989; and Ausubel, et al., eds., CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to TTG3 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at least 6 contiguous nucleotides of SEQ ID NO: 1, SEQ ID NO:2, SEQ ID NO:6 or SEQ ID NO:7, or a complement thereof. Optionally, the olignucleotides comprise at least 10, 15, 20, 25, 50, 100, 120, 150, 200, 300, 400, 500, 750 or more ontiguous nucleotides of SEQ ID NO: 1, SEQ ID NO:2, SEQ ID NO:6 or SEQ ID NO:7, or a complement thereof. Oligonucleotides may be chemically synthesized and may be used as probes.

In another embodiment, an isolated nucleic acid molecule of the invention includes a nucleic acid molecule that is a complement of the nucleotide sequence shown in SEQ ID NO: 1, SEQ ID NO:2, SEQ ID NO:6 or SEQ ID NO:7. In another embodiment, an isolated nucleic acid molecule of the invention comprises a nucleic acid molecule that is a complement of the nucleotide sequence shown in SEQ ID NO: 1, SEQ ID NO:2, SEQ ID NO:6 or SEQ ID NO:7, or a portion of these nucleotide sequence. A nucleic acid molecule that is complementary to the nucleotide sequence shown in SEQ ID NO: 1, SEQ ID NO:2, SEQ ID NO:6 or SEQ ID NO:7 is one that is sufficiently complementary to the nucleotide sequence shown in SEQ ID NO: 1, SEQ ID NO:2, SEQ ID NO:6 or SEQ ID NO:7 that it can hydrogen bond with little or no mismatches to the nucleotide sequence shown in SEQ ID NO: 1, SEQ ID NO:2, SEQ ID NO:6 or SEQ ID NO:7, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, SEQ ID NO:2, SEQ ID NO:6 or SEQ ID NO:7, e.g., a fragment that can be used as a probe or primer, or a fragment encoding a biologically active portion of TTG3. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety). A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a TTG3 polypeptide. Isoforms can be expressed in different tissues of the same organism as a result of, for example, alternative splicing of RNA. Alternatively, isoforms can be encoded by different genes. Homologous nucleotide sequences also include, but are not limited to, naturally occurring allelic variations and mutations of the nucleotide sequences set forth herein.

The nucleotide sequence determined from the cloning of the Arabidopsis thaliana TTG3 gene allows for the generation of probes and primers designed for use in identifying and/or cloning TTG3 homologues in other cell types, e.g., from other tissues, as well as TTG3 homologues from other plants. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, SEQ ID NO:2, SEQ ID NO:6, or SEQ ID NO:7; or an anti-sense strand nucleotide sequence of SEQ ID NO: 1, SEQ ID NO:2, SEQ ID NO:6, or SEQ ID NO:7; or of a naturally occurring mutant of SEQ ID NO: 1, SEQ ID NO:2, SEQ ID NO:6, or SEQ ID NO:7.

Probes based on the Arabidopsis thaliana, TTG3 nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TTG3 protein, such as by measuring a level of a TTG3-encoding nucleic acid in a sample of cells from a subject e.g., detecting TTG3 mRNA levels or determining whether a genomic TTG3 gene has been mutated or deleted.

A “polypeptide having a biologically active portion of TTG3” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of TTG3” can be prepared by isolating a portion of SEQ ID NO: 1, SEQ ID NO:2, SEQ ID NO:6 or SEQ ID NO:7 that encodes a RNA or a polypeptide having a TTG3 biological activity (biological activities of the TTG3 proteins are described below), expressing the encoded portion of TTG3 proteins (e.g., by recombinant expression in vitro) and assessing the activity of the encoded portion of TTG3. In another embodiment, a nucleic acid fragment encoding a biologically active portion of TTG3 includes one or more regions.

TTG3 Variants

The invention further encompasses nucleic acid molecules that differ from the nucleotide sequences shown in SEQ ID NO: 1, SEQ ID NO:2, SEQ ID NO:6 or SEQ ID NO:7 due to the degeneracy of the genetic code. These nucleic acids thus encode the same TTG3 protein as that encoded by the nucleotide sequence shown in SEQ ID NO: 1, SEQ ID NO:2, SEQ ID NO:6 or SEQ ID NO:7, e.g., the polypeptide of SEQ ID NO:4. In another embodiment, an isolated nucleic acid molecule of the invention has a nucleotide sequence encoding a protein having an amino acid sequence shown in SEQ ID NO:4.

In addition to the Arabidopsis thaliana, TTG3 nucleotide sequence shown in SEQ ID NO: 1, SEQ ID NO:2, SEQ ID NO:6 or SEQ ID NO:7, it will be appreciated by those skilled in the art that DNA sequence polymorphisms that lead to changes in the amino acid sequences of TTG3 may exist within a population (e.g., the plant). Such genetic polymorphism in the TTG3 gene may exist among individuals within a population due to natural allelic variation. As used herein, the terms “gene” and “recombinant gene” refer to nucleic acid molecules comprising an open reading frame encoding a TTG3 protein, preferably a plant TTG3 protein. Such natural allelic variations can typically result in 1-5% variance in the nucleotide sequence of the TTG3 gene. Any and all such nucleotide variations and resulting amino acid polymorphisms in TTG3 that are the result of natural allelic variation and that do not alter the functional activity of TTG3 are intended to be within the scope of the invention.

Moreover, nucleic acid molecules encoding TTG3 proteins from other species, and thus that have a nucleotide sequence that differs from the sequence of SEQ ID NO: 1, SEQ ID NO:2, SEQ ID NO:6 or SEQ ID NO:7 are intended to be within the scope of the invention. Nucleic acid molecules corresponding to natural allelic variants and homologues of the TTG3 cDNAs of the invention can be isolated based on their homology to the Arabidopsis thaliana TTG3 nucleic acids disclosed herein using the cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, SEQ ID NO:2, SEQ ID NO:6 or SEQ ID NO:7. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding TTG3 proteins derived from species other than Arabidopsis thaliana, or other related sequences (e.g., paralogs) can be obtained by low, moderate or high stringency hybridization with all or a portion of the particular sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different depending upon circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO: 1, SEQ ID NO:2, SEQ ID NO:6 or SEQ ID NO:7 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, SEQ ID NO:2, SEQ ID NO:6 or SEQ ID NO:7, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, SEQ ID NO:2, SEQ ID NO:6 or SEQ ID NO:7 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et aL (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the TTG3 sequence that may exist in the population, the skilled artisan will further appreciate that changes can be introduced by mutation into the nucleotide sequence of SEQ ID NO: 1, SEQ ID NO:2, SEQ ID NO:6 or SEQ ID NO:7, thereby leading to changes in the amino acid sequence of the encoded TTG3 protein, without altering the functional ability of the TTG3 protein. For example, nucleotide substitutions leading to amino acid substitutions at “non-essential” amino acid residues can be made in the sequence of SEQ ID NO: 1,SEQ ID NO:2, SEQ ID NO:6 or SEQ ID NO:7. A “non-essential” amino acid residue is a residue that can be altered from the wild-type sequence of TTG3 without altering the biological activity, whereas an “essential” amino acid residue is required for biological activity. For example, amino acid residues that are conserved among the TTG3 proteins of the present invention, are predicted to be particularly unamenable to alteration.

Another aspect of the invention pertains to nucleic acid molecules encoding TTG3 proteins that contain changes in amino acid residues that are not essential for activity. Such TTG3 proteins differ in amino acid sequence from SEQ ID NO:4, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO:4. Preferably, the protein encoded by the nucleic acid is at least about 80% homologous to S SEQ ID NO:4 more preferably at least about 90%, 95%, 98%, and most preferably at least about 99% homologous to SEQ ID NO:4.

An isolated nucleic acid molecule encoding a TTG3 protein homologous to the protein of SEQ ID NO:4 can be created by introducing one or more nucleotide substitutions, additions or deletions into the nucleotide sequence of SEQ ID NO: 1, SEQ ID NO:2, SEQ ID NO:6 or SEQ ID NO:7 such that one or more amino acid substitutions, additions or deletions are introduced into the encoded protein.

Mutations can be introduced into the nucleotide sequence of SEQ ID NO: 1, SEQ ID NO:2, SEQ ID NO:6 or SEQ ID NO:7 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in TTG3 is replaced with another amino acid residue from the same side chain family. Alternatively, in another embodiment, mutations can be introduced randomly along all or part of a TTG3 coding sequence, such as by saturation mutagenesis, and the resultant mutants can be screened for TTG3 biological activity to identify mutants that retain activity. Following mutagenesis of SEQ ID NO: 1, SEQ ID NO:2, SEQ ID NO:6 or SEQ ID NO:7 the encoded protein can be expressed by any recombinant technology known in the art and the activity of the protein can be determined.

In one embodiment, a mutant TTG3 protein can be assayed for (1) the ability to form protein:protein interactions with other TTG3 proteins, other cell-surface proteins, or biologically active portions thereof, (2) complex formation between a mutant TTG3 protein and a TTG3 receptor; (3) the ability of a mutant TTG3 protein to bind to an intracellular target protein or biologically active portion thereof; (e.g., avidin proteins); (4) the ability to bind TTG3 protein; or (5) the ability to specifically bind an anti-TTG3 protein antibody.

Antisense TTG3 Nucleic Acids

Another aspect of the invention pertains to isolated antisense nucleic acid molecules that are hybridizable to or complementary to the nucleic acid molecule comprising the nucleotide sequence of SEQ ID NO: 1, SEQ ID NO:2, SEQ ID NO:6 or SEQ ID NO:7, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire TTG3 coding strand, or to only a portion thereof. Nucleic acid molecules encoding fragments, homologs, derivatives and analogs of a TTG3 protein of SEQ ID NO: 4 or antisense nucleic acids complementary to a TTG3 nucleic acid sequence of SEQ ID NO: 1, SEQ ID NO:2, SEQ ID NO:6 or SEQ ID NO:7 are additionally provided, see for example SEQ ID NO:80.

In one embodiment, an antisense nucleic acid molecule is antisense to a “coding region” of the coding strand of a nucleotide sequence encoding TTG3. The term “coding region” refers to the region of the nucleotide sequence comprising codons which are translated into amino acid residues (e.g., the protein coding region of Arabidopsis thaliana TTG3 corresponds to SEQ ID NO: 3. Furthermore, the term coding may refer to a nucleic acid sequence that is transcribed but is not translated into a polypeptide. In this respect the term coding refers to the region of a nucleic acid sequence that is transcribed and produces a mature RNA molecule. Mature RNA molecules are produced as a result of an intron splicing process, wherein the introns are removed from the transcribed sequence. The spliced RNA can be refered to as a mature RNA. For example, nature RNA can be a mRNA (message RNA) or a tRNA (transfer RNA) or a ribozyme. RNAs that are detected as expressed sequence tags (EST) are considered mature RNAs. In another embodiment, the antisense nucleic acid molecule is antisense to a “noncoding region” of the coding strand of a nucleotide sequence encoding TTG3. The term “noncoding region” refers to 5′ and 3′ sequences which flank the coding region that are not translated into amino acids (i.e., also referred to as 5′ and 3′ untranslated regions).

Given the coding strand sequences encoding TTG3 disclosed herein (e.g., SEQ ID NO: 1, SEQ ID NO:2, SEQ ID NO:6 or SEQ ID NO:7), antisense nucleic acids of the invention can be designed according to the rules of Watson and Crick or Hoogsteen base pairing. The antisense nucleic acid molecule can be complementary to the entire coding region of TTG3 mRNA, but more preferably is an oligonucleotide that is antisense to only a portion of the coding or noncoding region of TTG3 mRNA. For example, the antisense oligonucleotide can be complementary to the region surrounding the translation start site of TTG3 mRNA, see for example SEQ ID NO:80. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are generated in situ such that they hybridize with or bind to cellular mRNA and/or genomic DNA encoding a TTG3 protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in applications.

Double Stranded RNA Inhibition (RNAi) by Hairpin Nucleic Acids

Another aspect of the invention pertains to the use of post transcriptional gene silencing (PTGS) to repress gene expression. Double stranded RNA can initiate the sequence specific repression of gene expression in plants and animals. Double stranded RNA is processed to short duplex oligomers of 21-23 nucleotides in length. These small interfering RNA's suppress the expression of endogenous and heterologous genes in a sequence specific manner (Fire et al. Nature 391:806-811, Carthew, Curr. Opin. in Cell Biol., 13:244-248, Elbashir et al., Nature 411:494-498). A RNAi suppressing construct can be designed in a number of ways, for example, transcription of a inverted repeat which can form a long hair pin molecule, inverted repeats separated by a spacer sequence that could be an unrelated sequence such as a portion of a β-glucuronidase gene GUS or an intron sequence. Transcription of sense and antisense strands by opposing promoters or cotranscription of sense and antisense genes.

TTG3 Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave TTG3 mRNA transcripts to thereby inhibit translation of TTG3 mRNA. A ribozyme having specificity for a TTG3-encoding nucleic acid can be designed based upon the nucleotide sequence of a TTG3 DNA disclosed herein (i.e., SEQ ID NO: 1, SEQ ID NO:2, SEQ ID NO:6 or SEQ ID NO:7). For example, a derivative of a Tetrahymena L-19 IVS RNA can be constructed in which the nucleotide sequence of the active site is complementary to the nucleotide sequence to be cleaved in a TTG3-encoding mRNA. See, e.g., Cech et al. U.S. Pat. No. 4,987,071; and Cech et al. U.S. Pat. No. 5,116,742. Alternatively, TTG3 mRNA can be used to select a catalytic RNA having a specific ribonuclease activity from a pool of RNA molecules. See, e.g., Bartel et al., (1993) Science 261:1411-1418.

Alternatively, TTG3 gene expression can be inhibited by targeting nucleotide sequences complementary to the regulatory region of the TTG3 (e.g., the TTG3 promoter and/or enhancers) to form triple helical structures that prevent transcription of the TTG3 gene in target cells. See generally, Helene. (1991) Anticancer Drug Des. 6: 569-84; Helene. et al. (1992) Ann. N.Y Acad. Sci. 660:27-36; and Maher (1992) Bioassays 14: 807-15.

In various embodiments, the nucleic acids of TTG3 can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g. DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of TTG3 can be used in therapeutic and diagnostic applications. For example, PNAs can be used as antisense or antigene agents for sequence-specific modulation of gene expression by, e.g., inducing transcription or translation arrest or inhibiting replication. PNAs of TTG3 can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of TTG3 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of TTG3 can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

TTG3 Promoters and Promoter Constructs

The invention provides previously unidentified promoter nucleic acid sequences isolated from the Arabidopsis thaliana (At) TTG3 gene. The term “promoter” refers to a region of DNA upstream from the translational start codon which is involved in recognition and binding of RNA polymerase and other proteins to initiate transcription. The TTG3 promoter sequence of the invention includes the nucleic acid sequence of SEQ ID NO:5 or SEQ ID NO:8. The TTG3 promoter sequences of the invention are typically identical to or show substantial sequence identity nucleic acid sequence depicted in SEQ ID NO:5 or SEQ ID NO:8. or fragments thereof. A TTG3 promoter sequence is at least 25%, 50%, 75%, 80%, 85%, 90%, 95%, 96%, 97%, 98% 99 identical to SEQ ID NO:5 or SEQ ID NO:8.

The TTG3 promoter sequence is 1215 nucleotides in length. All or part (i.e., fragment) of the TTG3 promoter may be used to specifically direct expression of a sequence or gene to plant tissue. Optionally, the TTG3 promoter contains additional nucleic acid sequences at the 5′ or 3′ end. The additional nucleic acid sequence is a coding sequence. Alternatively, the additional nucleic acid sequence is a non-coding sequence. For example, the promoter includes other transcriptional and translational regulatory nucleic acid sequences (also termed “control sequences”) that are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A TTG3 promoter sequence is less than 1215 nucleotides in length, e.g., less than or equal to 1100, 1000, 800, 750, 600, 625, 600, 550, 525, 500, 450, 433, 400, 200 nucleotides in length.

The TTG3 promoters are capable of conferring high levels (i.e., strong promoter) of transcription in plant tissue when used as a promoter for a heterologous coding sequence. As used herein, “promoter strength” refers to the level of promoter-regulated expression of a heterologous gene in a plant tissue or tissues, relative to a standard (a standard gene promoter, e.g., the 35S CaMV promoter or the CsVMV promoter). Expression levels is measured by linking the promoter to a suitable reporter gene such as GUS (beta.-glucuronidase). Expression of the reporter gene can be easily measured by fluorometric, spectrophotometric or histochemical assays.

Various modifications are made to the promoters of the invention to provide promoters with different properties (e.g., tissue specificity, promoter strength, and the like). For example, truncated forms of a TTG3 promoter are constructed by mapping restriction enzyme sites in the promoter and then using the constructed map to determine appropriate restriction enzyme cleavage to excise a subset of the sequence. The modified promoters are then inserted into a suitable vector and tested for their ability to drive expression of a marker gene. Tissue specificity of the modified promoters is tested in regenerated plants. An exemplary modified TTG3 promoter includes the nucleic acid of SEQ ID NO:5 which allows for expression of a gene of interest in the root tissue of a plant.

TTG3 promoters are isolated in a variety of ways know in the art. For example, TTG3 promoters are isolated from genomic DNA fragments encoding a TTG3 protein and which also contain sequences upstream from the sequence encoding the TTG3 protein. Genomic fragments encoding TTG3 proteins are isolated by methods known in the art. TTG3 promoter sequences are isolated by screening plant DNA libraries with oligonucleotide probes having sequences derived from the DNA sequence of the TTG3 promoter of SEQ ID NO:5 or SEQ ID NO:8. Other methods known to those of skill in the art can also be used to isolate plant DNA fragments containing TTG3 promoters. See Sambrook, et al. for a description of other techniques for the isolation of DNAs related to DNA molecules of known sequence. For instance, deletion analysis and a promoterless reporter gene (e.g., GUS) can be used to identify those regions which can drive expression of a structural gene. Sequences characteristic of promoter sequences can also be used to identify the promoter. Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions-80 to -100, there is typically a promoter element with a series of adenines surrounding the trinucleotide G (or T) N G. J. Messing et al., in Genetic Engineering in Plants, pp. 221-227 (Kosage, Meredith and Hollaender, eds. 1983).

The TTG3 promoter is useful in ligating or fusing (i.e., operably linked) to the 5′ end of one or more nucleic acid sequences (e.g., gene) thereby producing a TTG3 promoter—gene construct. The term “operably linked” as used herein refers to linkage of a promoter upstream from a DNA sequence such that the promoter mediates transcription of the DNA sequence. The TTG3 promoter is ligated in frame upstream of a sequence to be expressed. Downstream or 3′ of the sequence to be expressed may be suitable transcription termination signals, including a polyadenylation signal or other sequences found helpful in the processing of the 3′ mRNA terminus. The promoter sequence also includes transcribed sequences between the transcriptional start and the translational start codon. Optionally, the construct contains a nucleic acid encoding a reporter gene or a selectable marker ligated 3′ of the TTG3 promoter. The reporter/marker sequence provides a means to easily identify the cells expressing the sequences under control of the TTG3 promoter. For example, selectable marker genes encode a polypeptide that permits selection of transformed plant cells containing the gene by rendering the cells resistant to an amount of an antibiotic that would be toxic to non-transformed plant cells. Selectable marker genes include the neomycin phosphotransferase (nptII) resistance gene, hygromycin phosphotransferase (hpt), bromoxynil-specific nitrilase (bxn), phosphinothricin acetyltransferase enzyme (BAR) and the spectinomycin resistance gene (spt), wherein the selective agent is kanamycin, hygromycin, geneticin, the herbicide glufosinate-ammonium (“Basta”) or spectinomycin, respectively.

The nucleic acid sequences is heterologous (i.e., exogenous) to the promoter. Exogenous and heterologous, as used herein, denote a nucleic acid sequence which is not obtained from and would not normally form a part of the genetic make-up of the plant or the cell to be transformed, in its untransformed state. Foreign genes and sequences, for purposes of the present invention, are those which are not naturally occurring in the plant into which they are delivered. Portions of the above mentioned heterologous or exogenous sequences and foreign genes and sequences are of plant origin, however, the TTG3 promoter-gene construct forms a combination or variant not naturally occurring in the plant The nucleic acid encodes for a protein of interest or fragment thereof. The gene is for example a structural gene, an enzyme (e.g., farnesyl transferase, alpha or beta or CaaX prenyl protease), a chaperonin protein (e.g., HSP or Ras)), a scaffolding protein, or a transcriptional regulator. For example, the nucleic acid encodes for a gene capable of altering an agronomic trait such as disease resistance, herbicide resistance, environmental stress resistance or increased yield. Alteration of prenylation by increasing or decreasing farnesyl transferase, CaaX prenyl protease activity has been shown to elicit plants with altered agronomic traits. (See for example, PCT US 98/15664, US 03/26894, WO 02/097097 and WO 03/012116, each of which are incorporated by reference in their entireties)

The nucleic acid sequences are DNA, such as cDNA and genomic DNA or RNA, such as mRNA and tRNA. For example, the nucleic acid sequence is a non-translated mRNA molecule of a gene or fragment thereof that encodes a protein of interest. Non-translated mRNA includes, e.g., antisense, hairpin RNA, microRNA, or ribozymes. The non-translated mRNA may alter agronomic traits, including those identified above. Alternatively, the non-translated mRNA may prevent the translation of sequences which are detrimental to the plant.

The TTG3 promoter-gene contains one promoter nucleic acid sequence. Alternatively, the TTG3 promoter-gene construct contains 2, 3, 4, 5, or more promoter nucleic acid sequences. Optionally, the promoter sequences are linked together by a spacer. No particular length is implied by the term spacer. The spacer is less than 1000 nucleotides in length, e.g., less than or equal to 900, 800, 700, 500, 250, 100, 75, 50, 35, 25, or 10 nucleotides in length.

The promoter regulates expression of the nucleic acid sequence of interest constitutively. Alternatively, the promoter is inducible by a stimulus such as light or an environmental stress, such as drought, chilling stress, salt stress, a pathogen, a herbicide, or wounding. The terms “constitutive promoter” as used herein refer to a promoter which is capable of expressing operably linked DNA sequences in all tissues or nearly all tissues of a plant. The terms “inducible promoter”, as used herein, refer to plant promoters that are capable of selectively expressing operably linked DNA sequences at particular times in response to endogenous or external stimuli.

Also included in the invention are vectors containing the TTG3 promoter-gene constructs. Suitable plant expression vectors systems include tumor inducing (Ti) plasmid or portion thereof found in Agrobacterium, cauliflower mosaic virus (CaMV) DNA and vectors such as pBI121 .

For expression of TTG3 promoter gene construct in plants, the recombinant expression cassette will contain in addition to the TTG3 promoter and nucleic acid of interest, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Additional regulatory elements that may be connected to the TTG3 promoter gene construct for expression in plant cells include terminators, polyadenylation sequences, and nucleic acid sequences encoding signal peptides that permit localization within a plant cell or secretion of the protein from the cell. Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)) are useful in the invention.

A number of types of cells may act as suitable host cells for expression of the vectors. Plant host cells include cells from monocots and dicots. For example, plant cells include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutum and Glycine max.

TTG3 Polypeptides

A TTG3 polypeptide of the invention includes the protein whose sequence is provided in SEQ ID NO: 4. The invention also includes a mutant or variant protein any of whose residues may be changed from the corresponding residue shown in SEQ ID NO: 4 while still encoding a protein that maintains its TTG3-like activities and physiological functions, or a functional fragment thereof. In some embodiments, up to 20% or more of the residues may be so changed in the mutant or variant protein. In some embodiments, the TTG3 polypeptide according to the invention is a mature polypeptide.

In general, a TTG3-like variant that preserves TTG3-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated TTG3 proteins, and biologically active portions thereof, or derivatives, fragments, analogs or homologs thereof. Also provided are polypeptide fragments suitable for use as immunogens to raise anti-TTG3 antibodies. In one embodiment, native TTG3 proteins can be isolated from cells or tissue sources by an appropriate purification scheme using standard protein purification techniques. In another embodiment, TTG3 proteins are produced by recombinant DNA techniques. Alternative to recombinant expression, a TTG3 protein or polypeptide can be synthesized chemically using standard peptide synthesis techniques.

An “isolated” or “purified” protein or biologically active portion thereof is substantially free of cellular material or other contaminating proteins from the cell or tissue source from which the TTG3 protein is derived, or substantially free from chemical precursors or other chemicals when chemically synthesized. The language “substantially free of cellular material” includes preparations of TTG3 protein in which the protein is separated from cellular components of the cells from which it is isolated or recombinantly produced. In one embodiment, the language “substantially free of cellular material” includes preparations of TTG3 protein having less than about 30% (by dry weight) of non-TTG3 protein (also referred to herein as a “contaminating protein”), more preferably less than about 20% of non-TTG3 protein, still more preferably less than about 10% of non-TTG3 protein, and most preferably less than about 5% non-TTG3 protein. When the TTG3 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TTG3 protein in which the protein is separated from chemical precursors or other chemicals that are involved in the synthesis of the protein. In one embodiment, the language “substantially free of chemical precursors or other chemicals” includes preparations of TTG3 protein having less than about 30% (by dry weight) of chemical precursors or non-TTG3 chemicals, more preferably less than about 20% chemical precursors or non-TTG3 chemicals, still more preferably less than about 10% chemical precursors or non-TTG3 chemicals, and most preferably less than about 5% chemical precursors or non-TTG3 chemicals.

Biologically active portions of a TTG3 protein include peptides comprising amino acid sequences sufficiently homologous to or derived from the amino acid sequence of the TTG3 protein, e.g., the amino acid sequence shown in SEQ ID NO: 4 that include fewer amino acids than the full length TTG3 proteins, and exhibit at least one activity of a TTG3 protein, e.g substrate binding. Typically, biologically active portions comprise a domain or motif with at least one activity of the TTG3 protein. A biologically active portion of a TTG3 protein can be a polypeptide which is, for example, 10, 25, 50, 100 or more amino acids in length.

A biologically active portion of a TTG3 protein of the present invention may contain at least one of the above-identified domains conserved between the TTG3 proteins, e.g. Moreover, other biologically active portions, in which other regions of the protein are deleted, can be prepared by recombinant techniques and evaluated for one or more of the functional activities of a native TTG3 protein.

A biologically active portion or a. TTG3 protein can be the N-terminal domain of the TTG3 polypeptide. Alternatively, a biologically active portion or a TTG3 protein can be the C-terminal domain of the TTG3 polypeptide. Preferably, the biologically active portion comprises at least 75 amino acids of the C-terminal domain. More preferably, the biologically active portion comprises at least 25 amino acids of the C-terminal domain. Most preferably, the biologically active portion comprises at least 10 amino acids of the C-terminal.

In an embodiment, the TTG3 protein has an amino acid sequence shown in SEQ ID NO: 4. In other embodiments, the TTG3 protein is substantially homologous to SEQ ID NO: 4 and retains the functional activity of the protein of SEQ ID NO: 4, yet differs in amino acid sequence due to natural allelic variation or mutagenesis, as described in detail below. Accordingly, in another embodiment, the TTG3 protein is a protein that comprises an amino acid sequence at least about 45% homologous to the amino acid sequence of S SEQ ID NO: 4 and retains the functional activity of the TTG3 proteins of SEQ ID NO: 4.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (ie., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits-a degree of identity preferably of at least 70%, 75%, 80%, 85%, 90%, 95%, 98%, or 99%, with the CDS (encoding) part of the DNA sequence shown in SEQ ID NO:1 or SEQ ID NO:2, SEQ ID NO:6or SEQ ID NO:7.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i. e., the window size), and multiplying the result by 100 to yield the percentage of positive residues.

TTG3 Recombinant Expression Vectors and Host Cells

Another aspect of the invention pertains to vectors, preferably expression vectors, containing a nucleic acid encoding a TTG3 protein, gene or derivatives, fragments, analogs or homologs thereof. As used herein the term expression vector includes vectors which are designed to provide transcription of the nucleic acid sequence. The gene may encode a RNA molecule that has biological activity as the RNA molecule. Additionally, the transcribed nucleic acid may be translated into a polypeptide or protein product. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). Examples of suitable promoters include for example constitutive promoters, ABA inducible promoters, tissue specific promters or guard cell specific promoters. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TTG3 proteins, mutant forms of TTG3 proteins, fusion proteins, etc.).

The recombinant expression vectors of the invention can be designed for expression of TTG3 genes or TTG3 proteins in prokaryotic or eukaryotic cells. For example, TTG3 genes or TTG3 proteins can be expressed in bacterial cells such as Escherichia coli, insect cells (using baculovirus expression vectors) yeast cells, plant cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of genes or proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the TTG3 expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). Alternatively, TTG3 can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In yet another embodiment, a nucleic acid of the invention is expressed in plants cells using a plant expression vector. Examples of plant expression vectors systems include tumor inducing (Ti) plasmid or portion thereof found in Agrobacterium, cauliflower mosaic virus (CAMV) DNA and vectors such as pBI121.

For expression in plants, the recombinant expression cassette will contain in addition to the TTG3 nucleic acids, a plant promoter region, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector. Examples of suitable promotors include promoters from plant viruses such as the 35S promoter from cauliflower mosaic virus (CaMV). Odell, et al., Nature, 313: 810-812 (1985). and promoters from genes such as rice actin (McElroy, et al., Plant Cell, 163-171 (1990)); ubiquitin (Christensen, et al., Plant Mol. Biol., 12: 619-632 (1992); and Christensen, et al., Plant Mol. Biol., 18: 675-689 (1992)); pEMU (Last, et al., Theor. Appl. Genet., 81: 581-588 (1991)); MAS (Velten, et al., EMBO J., 3: 2723-2730 (1984)); maize H3 histone (Lepetit, et al., Mol. Gen. Genet., 231: 276-285 (1992); and Atanassvoa, et al., Plant Journal, 2(3): 291-300 (1992)), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as, Rsyn7, SCP and UCP promoters, ribulose-1,3-diphosphate carboxylase, fruit-specific promoters, heat shock promoters, seed-specific promoters and other transcription initiation regions from various plant genes, for example, include the various opine initiation regions, such as for example, octopine, mannopine, and nopaline. Additional regulatory elements that may be connected to a TTG3 encoding nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements TTG3 gene are known, and include, but are not limited to, 3′ termination and/or polyadenylation regions such as those of the Agrobacterium tumefaciens nopaline synthase (nos) gene (Bevan, et al., Nucl. Acids Res., 12: 369-385 (1983)); the potato proteinase inhibitor II (PINII) gene (Keil, et al., Nucl. Acids Res., 14: 5641-5650 (1986) and hereby incorporated by reference); and An, et al., Plant Cell, 1: 115-122 (1989)); and the CaMV 19S gene (Mogen, et al., Plant Cell, 2: 1261-1272 (1990)).

Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DèLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)) are useful in the invention.

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art. Especially useful in connection with the nucleic acids of the present invention are expression systems which are operable in plants. These include systems which are under control of a tissue-specific promoter, as well as those which involve promoters that are operable in all plant tissues.

Organ-specific promoters are also well known. For example, the patatin class I promoter is transcriptionally activated only in the potato tuber and can be used to target gene expression in the tuber (Bevan, M., 1986, Nucleic Acids Research 14:4625-4636). Another potato-specific promoter is the granule-bound starch synthase (GBSS) promoter (Visser, R. G. R, et al., 1991, Plant Molecular Biology 17:691-699). Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, P., 1986, Trans. R. Soc. London B314:343).

For in situ production of the antisense mRNA of TTG3, those regions of the TTG3 gene which are transcribed into GST mRNA, including the untranslated regions thereof, are inserted into the expression vector under control of the promoter system in a reverse orientation. The resulting transcribed mRNA is then complementary to that normally produced by the plant.

The resulting expression system or cassette is ligated into or otherwise constructed to be included in a recombinant vector which is appropriate for plant transformation. The vector may also contain a selectable marker gene by which transformed plant cells can be identified in culture. Usually, the marker gene will encode antibiotic resistance. These markers include resistance to G418, hygromycin, bleomycin, kanamycin, and gentamicin. After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

A host cell of the invention, such as a prokaryotic or eukaryotic host cell in culture, can be used to produce (i.e., express) a polypeptide of the invention encoded in an open reading frame of a polynucleotide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

A number of types of cells may act as suitable host cells for expression of a polypeptide encoded by an open reading frame in a polynucleotide of the invention. Plant host cells include, for example, plant cells that could function as suitable hosts for the expression of a polynucleotide of the invention include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutum and Glycine max.

Alternatively, it may be possible to produce a polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain a functional polypeptide, if the polypeptide is of sufficient length and conformation to have activity. Such covalent attachments may be accomplished using known chemical or enzymatic methods.

A polypeptide may be prepared by culturing transformed host cells under culture conditions suitable to express the recombinant protein. The resulting expressed polypeptide or protein may then be purified from such culture (e.g., from culture medium or cell extracts) using known purification processes, such as gel filtration and ion exchange chromatography. The purification of the polypeptide or protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, a polypeptide or protein may also be expressed in a form which will facilitate purification. For example, it may be expressed as a fusion protein containing a six-residue histidine tag. The histidine-tagged protein will then bind to a Ni-affinity column. After elution of all other proteins, the histidine-tagged protein can be eluted to achieve rapid and efficient purification. One or more reverse-phase high performance liquid chromatography (RP-HPLC) steps employing hydrophobic RP-HPLC media, e.g., silica gel having pendant methyl or other aliphatic groups, can be employed to further purify a polypeptide. Some or all of the foregoing purification steps, in various combinations, can also be employed to provide a substantially homogeneous isolated recombinant polypeptide. The protein or polypeptide thus purified is substantially free of other plant proteins or polypeptides and is defined in accordance with the present invention as “isolated.”

Transformed Plants Cells and Transgenic Plants

The invention includes a protoplast, plants cells, plant tissue and plants (e.g., monocots and dicots transformed with a TTG3 nucleic acid (i.e, sense or antisense), a vector containing a TTG3 nucleic acid (i. e, sense or antisense)or an expression vector containing a TTG3 nucleic acid (i.e, sense or antisense). As used herein, “plant” is meant to include not only a whole plant but also a portion thereof (i. e., cells, and tissues, including for example, leaves, stems, shoots, roots, flowers, fruits and seeds).

The plant can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

The invention also includes cells, tissues, including for example, leaves, stems, shoots, roots, flowers, fruits and seeds and the progeny derived from the tranformed plant.

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88 and Andrew Bent in, Clough S J and Bent A F, 1998. Floral dipping: a simplified method for Agrobacterium-mediated transformation of Arabidopsis thaliana. The methods chosen vary with the host plant, and include chemical transfection methods such as calcium phosphate, polyethylene glycol (PEG) transformation, microorganism-mediated gene transfer such as Agrobacterium (Horsch, et al., Science, 227: 1229-31 (1985)), electroporation, protoplast transformation, micro-injection, flower dipping and biolistic bombardment.

Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10: 1-32 (1991). Descriptions of the Agrobacterium vector systems and methods for Agrobacterium-mediated gene transfer are provided in Gruber et al., supra; and Moloney, et al, Plant Cell Reports, 8: 238-242 (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the plant has both developing flowers and open flowers. The plants are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

Direct Gene Transfer

A generally applicable method of plant transformation is microprojectile-mediated transformation, where DNA is carried on the surface of microprojectiles measuring about 1 to 4 mu.m. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)). Another method for physical delivery of DNA to plants is sonication of target cells as described in Zang, et al., BioTechnology, 9: 996-996 (1991). Alternatively, liposome or spheroplast fusions have been used to introduce expression vectors into plants. See, for example, Deshayes, et al., EMBO J., 4: 2731-2737 (1985); and Christou, et al., Proc. Nat'l. Acad. Sci. (USA), 84: 3962-3966 (1987). Direct uptake of DNA into protoplasts using CaCl.sub.2 precipitation, polyvinyl alcohol or poly-L-ornithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int;l. Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994).

Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection.

Aerosol Beam Microinjection

Alternatively, nucleic acid molecules can be introduced into cells by an aerosol beam. Aerosol beam technology is used to accelerate wet or dry particles to speeds enabling the particles to penetrate living cells. Aerosol particles suspended in an inert gas are accelerated to very high velocity durig the jet expansion of the gas as it passes from a region of higher gas pressure to a region of lower gas pressure through a small orifice. The accelerated particles are positioned to impact a preferred target, for example a plant cell. The particles are constructed as droplets of a sufficiently small size so that the cell survives the penetration. See for example U.S. Pat. No. 5,240,842 and U.S. Application 20010026941.

Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the transgenic plant with a plant from a second taxon; (2) recovering reproductive material from the progeny of the cross; and (3) growing transgenic plants from the reproductive material. Where desirable or necessary, the agronomic characteristics of the second taxon can be substantially preserved by expanding this method to include the further steps of repetitively: (1) backcrossing the transgenic progeny with non-transgenic plants from the second taxon; and (2) selecting for expression of an associated marker gene among the progeny of the backcross, until the desired percentage of the characteristics of the second taxon are present in the progeny along with the gene or genes imparting marker gene trait.

By the term “taxon” herein is meant a unit of botanical classification. It thus includes, genus, species, cultivars, varieties, variants and other minor taxonomic groups which lack a consistent nomenclature.

Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregant and can transmit the TTG3 gene and its activity to its progeny. A more preferred transgenic plant is homozygous for the gene, and transmits that gene to all of its offspring on sexual mating. Seed from a transgenic plant may be grown in the field or greenhouse, and resulting sexually mature transgenic plants are self-pollinated to generate true breeding plants. The progeny from these plants become true breeding lines that are evaluated for increased expression of the TTG3 transgene.

Method of Producing Transgenic Plants

Also included in the invention are methods of producing a transgenic plant. The method includes introducing into one or more plant cells a compound that alters TTG3 expression or activity in the plant to generate a transgenic plant cell and regenerating a transgenic plant from the transgenic cell. In some aspects the compound increases TTG3 expression or activity. Alternatively, the compound decreases TTG3 expression or activity. The compound can be, e.g., (i) a TTG3 polypeptide; (ii) a TTG3 nucleic acid; (iii) a nucleic acid that increases expression of a TTG3 nucleic acid ; (iv) a nucleic acid that decreases the expression of a TTG3 ; (v) a TTG3 antisense nucleic acid and derivatives, fragments, analogs and homologs thereof. A nucleic acid that increases expression of a TGG3 nucleic acid includes, e.g., promoters, enhancers. The nucleic acid can be either endogenous or exogenous. Preferably, the compound is a TTG3 polypeptide or a TTG3 nucleic acid. For example the compound comprises the nucleic acid sequence of SEQ ID NO: 1, 2, 6, or 7 or fragement thereof. Alternatively, the compound is a TTG3 antisense nucleic acid. Preferrably, the compound is a TTG3 nucleic acid sequence endogenous to the species being transformed. Alternatively, the compound is a TTG3 nucleic acid sequence exogenous to the species being transformed.

In various aspects the transgenic plant has an altered phenotype as compared to a wild type plant (i. e., untransformed). By altered phenotype is meant that the plant has a one or more characteristic that is different from the wild type plant. For example, when the transgenic plant has been contacted with a compound that decreases the expression or activity of a TTG3 nucleic acid the plant has a phentypes such as increased seed oil content, decreased seed fiber content, reduced trichome production, altered trichome structure, transparent testa, reduced anthocyanin production reduced proanthocyaninidin production and reduced flavanol production compared to a wild type plant. Alternatively, when the the transgenic plant has been contacted with a compound that increases the expression or activity of a TTG3 nucleic acid the plant has a phentypes such as decreased seed oil content, increased seed fiber content, normal or increases trichome production, trichome structure, anthocyanin production, proanthocyaninidin production and flavanol production compared to a wild type plant.

The plant can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

Methods or Identification of Transformation

The present invention also provides methods visually identifying plants transformed with a nucleotide sequence (e.g., a heterologous gene). A TTG3 nucleic acid or fragment thereof is transformed into a plant that exhibits an abnormal (i.e., ttg3)phenotype for a morphological marker. If the transformation of the plant is successful, the progeny of the transformed plant will exhibit a normal phenotype. Alternatively, a TTG3 anti-sense nucleic acid or fragment thereof is transformed into a plant that exhibits a normal (e.g., wildtype) phenotype of a morphological marker. If the transformation of the plant is successful, the progeny of the transformed plant will exhibit an abnormal (i.e., ttg3)phenotype for a morphological marker. Optionally, the plant is further transformed with a second nucleic acid encoding a gene of interest. The second nucleic acid sequence is operably linked to a promoter that is functional in a plant cell. The second nucleic acid sequence is preferably linked to the TTG3 construct in that it is on the same vector. Alternatively, the second nucleic acid sequence is not linked to the TTG3 construct in that it is not on the same vector.

Morphological markers useful in the present invention are those which, when normally expressed (i.e, in the wild type form) cause the plant to have a visually observable phenotypical characteristic (e.g., when the morphological marker is normally expressed, the plant has trichomes, or the plant is of a particular size, or the plant has a particular shape of leaf, or the plant forms a crown gall, etc). However, when these morphological markers are mutated, the mutation causes the plant to exhibit visually observable characteristics that differ from the normal phenotype (i.e., the visually observable characteristic encoded or correlated with the morphological marker is abnormal when the morphological marker is mutated). The morphological marker is for example trichome production, seed oil content, seed fiber content, anthocyanin production, proanthocyaninidin production or flavanol production. For example, a plant that normally has trichomes does not have trichomes or has fewer trichomes when the morphological marker is mutated; a plant of normal size is dwarfed when the morphological marker is mutated; a plant that has a certain shape of leaf has a different shape of leaf when the morphological marker is mutated.

A “mutation” as used herein, may be an addition, insertion, deletion or substitution of one or more nucleotides in the morphological marker, which may cause the addition, insertion, deletion or substitution,of one or more amino acids in the polypeptide encoded by the nucleic acid sequence of the morphological marker. Mutations in the morphological markers of the present invention may lead to the creation of stop codons resulting in truncated polypeptides; removal of stop codons resulting in extended polypeptides; or a frameshift resulting in a polypeptide lacking the function of the polypeptide encoded by the gene.

The term “heterologous” is used to indicate that a nucleic acid sequence (i.e., a gene) or a protein has a different natural origin with respect to its present host (i.e., a cell or plant that into which it is transformed). “Heterologous” is also used to indicate that one or more of the domains present in a protein differ in their natural origin with respect to other domains present. “Homologous” is used to indicate that a nucleic acid sequence or a protein is of the same natural origin as its present host (i.e., a cell or plant that into which it is transformed). “Expression” refers to the transcription and translation of a structural heterologous nucleic acid to yield the encoded protein. When Agrobacterium-mediated transformation is used in the practice of the invention, the heterologous nucleic acid to be expressed is preferably incorporated into the T-region and is flanked by T-DNA border sequences of the Agrobacterium vector. Any heterologous gene or nucleic acid that is desired to be expressed in a plant is suitable for the practice of the present invention. Heterologous genes to be transformed and expressed in the plants of the present invention include but are not limited to genes that encode resistance to diseases and insects, genes conferring nutritional value, genes conferring antifungal, antibacterial or antiviral activity, and the like. Alternatively, therapeutic (e.g., for veterinary or medical uses) or immunogenic (e.g., for vaccination) peptides and proteins can be expressed in plants transformed with the according to the present invention. Likewise, the transfer of any nucleic acid for controlling gene expression in a plant is contemplated as an aspect of the present invention. For example, the nucleic acid to be transferred can encode an antisense oligonucleotide. Alternately, plants may be transformed with one or more genes to reproduce enzymatic pathways for chemical synthesis or other industrial processes. Heterologous nucleic acids useful in the present invention may be naturally occurring and may be obtained from prokaryotes or eukaryotes (e.g., bacteria, fungi, yeast, viruses, plants, insects, and mammals), or the nucleic acids may be synthesized in whole or in part.

Screening Methods

The isolated nucleic acid molecules of the invention can be used to express TTG3 protein (e.g., via a recombinant expression vector in a host cell), to detect TTG3 mRNA (e.g., in a biological sample) or a genetic lesion in a TTG3 gene, and to modulate TTG3 activity, as described further, below. In addition, the TTG3 proteins can be used to screen compounds that modulate the TTG3 protein activity or expression. In addition, the anti-TTG3 antibodies of the invention can be used to detect and isolate TTG3 proteins and modulate TTG3 activity.

The invention provides a method (also referred to herein as a “screening assay”) for identifying modulators, ie., candidate or test compounds or agents (e.g., peptides, peptidomimetics, small molecules or other drugs) that bind to TTG3 proteins or have a stimulatory or inhibitory effect on, e.g., TTG3 protein expression or TTG3 protein activity. The invention also includes compounds identified in the screening assays described herein.

In one embodiment, the invention provides assays for screening candidate or test compounds which bind to a TTG3 nucleic acid, TTG3 protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145. A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fugal, bacterial, or algal extracts, are known in the art and can be screened with any of the assays of the invention. Examples of methods for the synthesis of molecular libraries can be found in the art, for example in: DeWitt, et al., 1993. Proc. Natl. Acad Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.). In one embodiment, an assay is a cell-based assay in which a cell which expresses a TTG3 nucleic acid, TTG3 protein or polypeptide, or a biologically-active portion thereof, is contacted with a test compound and the ability of the test compound to bind to a TTG3 nucleic acid, TTG3 protein or polypeptide determined. The cell, for example, can be of mammalian origin, plant cell or a yeast cell. Determining the ability of the test compound to bind to the TTG3 nucleic acid, TTG3 protein or polypeptide can be accomplished, for example, by coupling the test compound with a radioisotope or enzymatic label such that binding of the test compound to the TTG3 nucleic acid, TTG3 protein or polypeptideor biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a TTG3 nucleic acid, TTG3 protein or polypeptide, or a biologically-active portion thereof, with a known compound which binds TTG3 to form an assay mixture, contacting the assay mixture with a test compound, and determining the ability of the test compound to interact with a TTG3 protein, wherein determining the ability of the test compound to interact with a TTG3 nucleic acid, TTG3 protein or polypeptide comprises determining the ability of the test compound to preferentially bind to TTG3 nucleic acid, TTG3 protein or polypeptide or a biologically-active portion thereof as compared to the known compound.

In another embodiment, an assay is a cell-based assay comprising contacting a cell expressing a TTG3 nucleic acid, TTG3 protein or polypeptide, or a biologically-active portion thereof, with a test compound and determining the ability of the test compound to modulate (e.g., stimulate or inhibit) the activity of the TTG3 nucleic acid, TTG3 protein or polypeptide or biologically-active portion thereof. Determining the ability of the test compound to modulate the activity of TTG3 nucleic acid, TTG3 protein or polypeptide or a biologically-active portion thereof can be accomplished, for example, by determining the ability of the TTG3 nucleic acid, TTG3 protein or polypeptide protein to bind to or interact with a TTG3 target molecule. As used herein, a “target molecule” is a molecule with which a TTG3 nucleic acid, TTG3 protein or polypeptide binds or interacts in nature, for example, a molecule on the surface of a cell which expresses a TTG3 interacting protein, a molecule on the surface of a second cell, a molecule in the extracellular milieu, a molecule associated with the internal surface of a cell membrane or a cytoplasmic molecule. A TTG3 target molecule can be a non-TTG3 molecule or a TTG3 nucleic acid, TTG3 protein or polypeptide of the invention In one embodiment, a TTG3 target molecule is a component of a signal transduction pathway that facilitates transduction of an extracellular signal (e.g. a signal generated by binding of a compound to a membrane-bound molecule) through the cell membrane and into the cell. The target, for example, can be a second intercellular protein that has catalytic activity or a protein that facilitates the association of downstream signaling molecules with TTG3. Determining the ability of the TTG3 nucleic acid, TTG3 protein or polypeptide to bind to or interact with a TTG3 target molecule can be accomplished by one of the methods described above for determining direct binding. In one embodiment, determining the ability of the TTG3 nucleic acid, TTG3 protein or polypeptide to bind to or interact with a TTG3 target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a TTG3-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a TTG3 nucleic acid, TTG3 protein or polypeptide or biologically-active portion thereof with a test compound and determining the ability of the test compound to bind to the TTG3 nucleic acid, TTG3 protein or polypeptide or biologically-active portion thereof. Binding of the test compound to the TTG3 nucleic acid, TTG3 protein or polypeptide can be determined either directly or indirectly as described above. In one such embodiment, the assay comprises contacting the TTG3 nucleic acid, TTG3 protein or polypeptide or biologically-active portion thereof with a known compound which binds TTG3 to form an assay mixture, contacting the assay mixture with a test compound, and determining the ability of the test compound to interact with a TTG3 nucleic acid, TTG3 protein or polypeptide, wherein determining the ability of the test compound to interact with a TTG3 nucleic acid, TTG3 protein or polypeptide comprises determining the ability of the test compound to preferentially bind to TTG3 nucleic acid, TTG3 protein or polypeptide or biologically-active portion thereof as compared to the known compound.

In still another embodiment, an assay is a cell-free assay comprising contacting TTG3 nucleic acid, TTG3 protein or polypeptide or biologically-active portion thereof with a test compound and determining the ability of the test compound to modulate (e.g. stimulate or inhibit) the activity of the TTG3 nucleic acid, TTG3 protein or polypeptide or biologically-active portion thereof. Determining the ability of the test compound to modulate the activity of TTG3 can be accomplished, for example, by determining the ability of the TTG3 nucleic acid, TTG3 protein or polypeptide to bind to a TTG3 target molecule by one of the methods described above for determining direct binding. In an alternative embodiment, determining the ability of the test compound to modulate the activity of TTG3 protein can be accomplished by determining the ability of the TTG3 nucleic acid, TTG3 protein or polypeptide further modulate a TTG3 target molecule. For example, the catalytic/enzymatic activity of the target molecule on an appropriate substrate can be determined as described above.

In yet another embodiment, the cell-free assay comprises contacting the TTG3 nucleic acid, TTG3 protein or polypeptide or biologically-active portion thereof with a known compound which binds TTG3 nucleic acid, TTG3 protein or polypeptide to form an assay mixture, contacting the assay mixture with a test compound, and determining the ability of the test compound to interact with a TTG3 nucleic acid, TTG3 protein or polypeptide, wherein determining the ability of the test compound to interact with a TTG3 nucleic acid, TTG3 protein or polypeptide comprises determining the ability of the TTG3 nucleic acid, TTG3 protein or polypeptide to preferentially bind to or modulate the activity of a TTG3 target molecule.

The cell-free assays of the invention are amenable to use of both the soluble form or the membrane-bound form of TTG3 nucleic acid, TTG3 protein or polypeptide. In the case of cell-free assays comprising the membrane-bound form of TTG3 nucleic acid, TTG3 protein or polypeptide, it may be desirable to utilize a solubilizing agent such that the membrane-bound form of TTG3 nucleic acid, TTG3 protein or polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton®X-100, Triton®X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either TTG3 nucleic acid, TTG3 protein or polypeptide or its target molecule to facilitate separation of complexed from uncomplexed forms of one or both of the proteins, as well as to accommodate automation of the assay. Binding of a test compound to TTG3 nucleic acid, TTG3 protein or polypeptide or interaction of TTG3 nucleic acid, TTG3 protein or polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-TTG3 fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or TTG3 nucleic acid, TTG3 protein or polypeptide, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of TTG3 protein binding or activity determined using standard techniques.

Other techniques for immobilizing proteins on matrices can also be used in the screening assays of the invention. For example, either the TTG3 nucleic acid, TTG3 protein or polypeptide or its target molecule can be immobilized utilizing conjugation of biotin and streptavidin. Biotinylated TTG3 nucleic acid, TTG3 protein or polypeptide or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with TTG3 nucleic acid, TTG3 protein or polypeptide or target molecules, but which do not interfere with binding of the TTG3 nucleic acid, TTG3 protein or polypeptide to its target molecule, can be derivatized to the wells of the plate, and unbound target or TTG3 nucleic acid, TTG3 protein or polypeptide trapped in the wells by antibody conjugation. Methods for detecting such complexes, in addition to those described above for the GST-immobilized complexes, include immunodetection of complexes using antibodies reactive with the TTG3 nucleic acid, TTG3 protein or polypeptide or target molecule, as well as enzyme-linked assays that rely on detecting an enzymatic activity associated with the TTG3 protein or target molecule.

In another embodiment, modulators of TTG3 nucleic acid, TTG3 protein or polypeptide expression are identified in a method wherein a cell is contacted with a candidate compound and the expression of TTG3 mRNA or protein in the cell is determined. The level of expression of TTG3 mRNA or protein in the presence of the candidate compound is compared to the level of expression of TTG3 mRNA or protein in the absence of the candidate compound. The candidate compound can then be identified as a modulator of TTG3 mRNA or protein expression based upon this comparison. For example, when expression of TTG3 mRNA or protein is greater (i.e., statistically significantly greater) in the presence of the candidate compound than in its absence, the candidate compound is identified as a stimulator of TTG3 mRNA or protein expression. Alternatively, when expression of TTG3 mRNA or protein is less (statistically significantly less) in the presence of the candidate compound than in its absence, the candidate compound is identified as an inhibitor of TTG3 mRNA or protein expression. The level of TTG3 mRNA or protein expression in the cells can be determined by methods described herein for detecting TTG3 mRNA or protein.

In yet another aspect of the invention, the TTG3 nucleic acid, TTG3 protein or polypeptide can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with TTG3 (“TTG3-binding proteins” or “TTG3-bp”) and modulate TTG3 activity. Such TTG3-binding proteins are also likely to be involved in the propagation of signals by the TTG3 proteins as, for example, upstream or downstream elements of the TTG3 pathway.

The two-hybrid system is based on the modular nature of most transcription factors, which consist of separable DNA-binding and activation domains. Briefly, the assay utilizes two different DNA constructs. In one construct, the gene that codes for TTG3 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TTG3-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with TTG3.

In yet another aspect of the invention are methods which utilize the transgenic plants of the invention to identify TTG3-interacting components via genetic screening protocols. These components can be for example, regulatory elements which modify TTG3-gene expression, interacting proteins which directly modify TTG3 activity or interacting proteins which modify components of the same signal transduction pathway and thereby exert an effect on the expression or activity of TTG3. Briefly, genetic screening protocols are applied to the transgenic plants of the invention and in so doing identify related genes which are not identified using a wild type background for the screen. For example an activation tagged library (Weigel, et al., 2000. Plant Physiol. 122: 1003-1013), can be produced using the transgenic plants of the invention as the genetic background. Plants are then screened for altered phenotypes from that displayed by the parent plants. Alternative methods of generating libraries from the transgenic plants, of the invention can be used, for example, chemical or irradiation induced mutations, insertional inactivation or insertional activation methods.

In yet another aspect of the invention are methods which utilize the transgenic plants of the invention to identify functional homologues of TTG3 from a variety of species. The TTG3 gene can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus. Briefly, libraries can be made containing the genome of the plant one wishes to find a TTG3 gene from. The library is transformed into ttg3 mutants to create a population of transgenic lines. Transformed lines are screened and individules isolated that display a loss of a ttg3 phenotype. The inserted nucleic acid is isolated and characterized to identify the TTG3 gene. Alternatively, the library can be made as an expression library derived from cDNA library. An advantage of this functional approach is that TTG3 genes that have little similarity at the nucleotide sequence level can be identified based on function.

The invention further pertains to novel agents identified by the aforementioned screening assays and uses thereof.

### EXAMPLES

### Example 1

### Mutant Isolation

Arabidopsis plants were transformed with a T-DNA construct derived from the pBI121 vector. The vector carried a gene construct for the over-expression of a nucleic acid sequence.

Arabidopsis lines containing said T-DNA construct were grown under standard conditions, 22° C., 16 hour photoperiod and 200 ummol/m2/s. A plant line was identified as having a ttg3 phenotype and selected for further analysis. The plant was initially characterized by the lack of trichomes on the leaves, except for a few on the leaf margins, and by seeds that are yellow in color, indicative of altered tannin biosynthesis.

### Example 2

### T-DNA Construct has no Identifiable Contribution to the TTG3 Phenotype

From the screened population one line of sibling plants displayed the ttg3 phenotype. All other lines were morphologically indistinguishable from wild-type. The phenotype is therefore unrelated to the gene carried by the T-DNA construct, but rather believed to be the result of an insertional effect at the site of T-DNA integration into the host chromosome.

### Example 3

### Mutant Identification

The T-DNA insertion site was identified by genome walking as described by Lin et al. 2001 (Lin and Li, 2001). Genomic DNA was isolated from leaf tissues of the ttg3 mutant using Qiagen DNeasy Plant Mini Kit. Restriction digest using SnaBI was performed and the resulting fragments were annealed with an adapter formed by the sequences of the oligonucleotides identified by SEQ ID NO: 18 and SEQ ID NO: 19. Fragments were amplified using PCR and PCR primer pairs that were homologous to the adapter and to a sequence known to be present in the T-DNA insert. PCR was performed using primers identified-by SEQ ID NO:20 and SEQ ID NO:21. A nested set of primers was used in a second PCR reaction using the product of the first PCR reaction as template and primers identified by SEQ ID NO:22 and SEQ ID NO:23 to produce a 1 kb DNA fragment. Primers identified by SEQ ID NO:20 and SEQ ID NO:22 specifically bind to the adapter while the primers identified by SEQ ID NO:21 and SEQ ID NO:23 specifically bind to the T-DNA left border. A 1 kb fragment was produced, isolated and cloned into a pBluescript TA vector by TA cloning. The cloned fragment was sequenced using T7 and T3 promoter primers. The resulting sequence is identified by SEQ ID NO: 1.

### Example 4

### Sequence Analysis

The nucleic acid sequence of SEQ ID NO:1, SEQ ID NO:2, SEQ ID NO:6 or SEQ ID NO:7 was compared to the Arabidopsis database using the BLAST alignment tool (NCBI). Clones having accession numbers AC018664 and AC008262 were identified as having significant homology to SEQ ID NO:1. Both of these accession numbers represent results of Arabidopsis genomic sequencing projects. Accession number AC008262 is a genomic clone containing a fragment of chromosome 1 on BAC F4N2. Annotation of this sequence identifies a 366 bp coding open reading frame over a genomic region of 870 bp. The encoded polypeptide is predicted to be 121 amino acids in length (Protein Id: AAF2706 1.1) and has been determined solely based on sequence analysis alone rather than experimental evidence. BLAST analysis reported the sequence identity to SEQ ID NO:1 as 99%.

In the case of AC018664, the sequence is reported by BLAST analysis to be 99% identical to SEQ ID NO:1 however annotation does not identify the sequence as a gene or as a coding sequence having an encoded protein. Analysis of the sequence listing indicates a predicted nucleic acid coding sequence is included herein as SEQ ID NO:3 and the corresponding predicted amino acid sequence is included herein as SEQ ID NO:5 or SEQ ID NO:8.

Analysis of the nucleic acid sequence of SEQ ID NO:1, SEQ ID NO:2, SEQ ID NO:6 or SEQ ID NO:7 and comparison to the sequence contained in accession numbers AC018664 and AC008262 indicates that the site of T-DNA insertion in the ttg3 mutant was within a putative promoter region. This insertion inhibits the expression or activity of the TTG3 gene thereby resulting in the ttg3 phenotype. The sequence identified by SEQ ID NO: 1, SEQ ID NO:2, SEQ ID NO:6 or SEQ ID NO:7 and isolated from the ttg3 mutant is therefore homologous to that of a wild-type TTG3 Arabidopsis gene.
Sequences Producing Significant Alignments: (Bits)Score Value
gi|12325073|gb|AC018364.51|AC018364 Arabidopsis thaliana chr . . . 1354 0.0
gi|5757471|gb|AC008262.4|AC008262 Genomic sequence for Arab . . . 1354 0. 0

Further sequence analysis identified the EST sequences T44742, AA040949, H76582, AA728500, AV822679, CB260172 and the predicted open reading frame of AC008262 have homology to TTG3. EST sequences are derived from cDNA libraries and therefore represent mRNA transcripts. The EST sequences identified include sequence which, based on the TTG3 genomic sequence, would be predicted to be intron sequence. The ESTs cover the entire putative coding sequence predicted in accession number AC018364 and additionally, contain the predicted intron regions. This indicates that the introns are not spliced, or not spliced correctly, in a mature RNA molecule or the sequence is not translated and may represent a non-coding RNA molecule.

The transcribed sequence of TTG3 has been determined to be the sequence represented by SEQ ID NO:7. Data derived from EST sequence homology analysis, Northern blot analysis using short sequence specific radiolabeled probes to delineate transcribed from untranscribed sequence and RT-PCR analysis using region specific primers all predict a more 3′ transcription initiation and a more 3′ transcription termination relative to the sequence presented in SEQ ID NO:1.

| SEQ ID NO:1 | |
|---|---|
| aaattctttacttaccaatccggtggagacacgtgagcccctatggcaat | |
| aaacttataaccttcttccgccactgcctatcacaacacctattagcttt | |
| gatgtgtggtggacagtggacttgtaatgATGGTGAGTGGCCTATATTCA | |
| TTATTCTTTTTAGAATCATACAAGAGTTTTTGCTTAAAATGTTTATCGAG | |
| TCACGGCTATTATATTTTTTCTGTTACTATTgtattcattttattcttaa | |
| agacaaaattaaaatgactctggccctctctctcttctctctctcaaaat | |
| tttcagAGAGAGAGATTGACATCAAACCAAAATTTTCGACGACGGTAAAT | |
| TGATGTCGTCGGTGATGGTTGAATTGCCGTCCGGTGTAGTATCCGGCTTT | |
| CGTCTGACATATATTGGCTTCCACAGCAGTACATGgtcggcccttgtcta | |
| gcatcatcggctctttcagcggtgatggctagctcacggtgttaatccgg | |
| cgtagttcggttttcttttccttttggtttatatcacggttacgtttcga | |
| ccgttgattgtctttagataagaattcattgggtatgtgtgttggtgatg | |
| gcgttggttttcagtccaagcttgcttaattagatctaattccaacctat | |
| cctttgagtttaaggtttacggataataattaggcagtttgcgtttttga | |
| gatgaagattcaaagctctttcctcagcttattatgattgcctttagctg | |
| tatctcttatattcaagcttcgattgggtctaagtaacagaggtggttaa | |
| tcgaagatcttttttctcttttgagattaatagatccgtattagatcctt | |
| acttctgttttgtagTTGTTGGAAAAATTCAGATGAAGCTAATCTTCGAG | |
| TTTCATTCGACTTAGTTTCAGTTTCCGGCAGAATCGATGGCTTCACGGCG | |
| AAGGATGATAGATGGAAAACTTTCGTCTCCTCAGGTTACATGTGTATTCG | |
| ATGCTAA | |
| Predicted TTG3 SEQ ID NO:1 + FULLtranscribed (+7) | |
| SEQ ID NO:2 | |
| aaattctttacttaccaatccggtggagacacgtgagcccctatggcaat | |
| aaacttataaccttcttccgccactgcctatcacaacacctattagcttt | |
| gatgtgtggtggacagtggacttgtaatgatggtgagtggcctatattca | |
| ttattctttttagaatcatacaagagtttttgcttaaaatgtttatcgag | |
| tcacggctattatattttttctgttactattgtattcattttATTCTTAA | |
| AGACAAAATTAAAATGACTCTGGCCCTCTCTCTCTTCTCTCTCTCAAAAT | |
| TTTCAGAGAGAGAGATTGACATCAAACCAAAATTTTCGACGACGGTAAAT | |
| TGATGTCGTCGGTGATGGTTGAATTGCCGTCCGGTGTAGTATCCGGCTTT | |
| CGTCTGACATATATTGGCTTCCACAGCAGTACATGGTCGGCCCTTGTCTA | |
| GCATCATCGGCTCTTTCAGCGGTGATGGCTAGCTCACGGTGTTAATCCGG | |
| CGTAGTTCGGTTTTCTTTTCCTTTTGGTTTATATCACGGTTACGTTTCGA | |
| CCGTTGATTGTCTTTAGATAAGAATTCATTGGGTATGTGTGTTGGTGATG | |
| GCGTTGGTTTTCAGTCCAAGCTTGCTTAATTAGATCTAATTCCAACCTAT | |
| CCTTTGAGTTTAAGGTTTACGGATAATAATTAGGCAGTTTGCGTTTTTGA | |
| GATGAAGATTCAAAGCTCTTTCCTCAGCTTATTATGATTGCCTTTAGCTG | |
| TATCTCTTATATTCAAGCTTCGATTGGGTCTAAGTAACAGAGGTGGTTAA | |
| TCGAAGATCTTTTTTCTCTTTTGAGATTAATAGATCCGTATTAGATCCTT | |
| ACTTCTGTTTTGTAGTTGTTGGAAAAATTCAGATGAAGCTAATCTTCGAG | |
| TTTCATTCGACTTAGTTTCAGTTTCCGGCAGAATCGATGGCTTCACGGCG | |
| AAGGATGATAGATGGAAAACTTTCGTCTCCTCAGGTTACATGTGTATTCG | |
| ATGCTAACACGTGTCTGAGATATATTAGCTTGATCCAATCTTCTTCTCTA | |
| AATGTAATGTTGGGCCAGTTGGACTTAAAATAGTCTCTGTAAACCGTTTT | |
| ATGTTGTTGGGCTTTTGCTCGTTCGTTGTATGTTATGTTCATCTTAATAA | |
| AAATCGTCACTTGTG | |
| Original rescue seq. | |
| SEQ ID NO:6 | |
| atgATGGTGAGTGGCCTATATTCATTATTCTTTTTAGAATCATACAAGAG | |
| TTTTTGCTTAAAATGTTTATCGAGTCACGGCTATTATATTTTTTCTGTTA | |
| CTATTgtattcattttattcttaaagacaaaattaaaatgactctggccc | |
| tctctctcttctctctctcaaaattttcagAGAGAGAGATTGACATCAAA | |
| CCAAAATTTTCGACGACGGTAAATTGATGTCGTCGGTGATGGTTGAATTG | |
| CCGTCCGGTGTAGTATCCGGCTTTCGTCTGACATATATTGGCTTCCACAG | |
| CAGTACATGgtcggcccttgtctagcatcatcggctctttcagcggtgat | |
| ggctagctcacggtgttaatccggcgtagttcggttttcttttccttttg | |
| gtttatatcacggttacgtttcgaccgttgattgtctttagataagaatt | |
| cattgggtatgtgtgttggtgatggcgttggttttcagtccaagcttgct | |
| taattagatctaattccaacctatcctttgagtttaaggtttacggataa | |
| taattaggcagtttgcgtttttgagatgaagattcaaagctctttcctca | |
| gcttattatgattgcctttagctgtatctcttatattcaagcttcgattg | |
| ggtctaagtaacagaggtggttaatcgaagatcttttttctcttttgaga | |
| ttaatagatccgtattagatccttacttctgttttgtagTTGTTGGAAAA | |
| ATTCAGATGAAGCTAATCTTCGAGTTTCATTCGACTTAGTTTCAGTTTCC | |
| GGCAGAATCGATGGCTTCACGGCGAAGGATGATAGATGGAAAACTTTCGT | |
| CTCCTCAGGTTACATGTGTATTCGATGCTAA | |
| TTG3-B cDNA sequence Columbia (−7) Rescue seq B | |
| SEQ ID NO:7 | |
| attcttaaagacaaaattaaaatgactctggccctctctctcttctctct | |
| ctcaaaattttcagagagagaatcaaaccaaaattttcgacgacggtaaa | |
| ttgatgtcgtcggtgatggttgaattgccgtccggtgtagtatccggctt | |
| tcgtctgacatatattggcttccacagcagtacatggtcggcccttgtct | |
| agcatcatcggctctttcagcggtgatggctagctcacggtgttaatccg | |
| gcgtagttcggttttcttttccttttggtttatatcacggttacgtttcg | |
| accgttgattgtctttagatcagaattcattgggtatgtgtgttggtgat | |
| ggcgttggttttcagtccaagcttgcttaattagatctaattccaaccta | |
| tcctttgagtttaaggtttacggataataattaggcagtttgcgtttttg | |
| agatgaagattcaaagctctttcctcagcttattatgattgcctttagct | |
| gtatctcttatattcaagcttcgattgggtctaagtaacagaggtggtta | |
| atcgaagatcttttttctcttttgagattaatagatccgtattagatcct | |
| tacttctgttttgtagttgttggaaaaattcagatgaagctaatcttcga | |
| gtttcattcgacttagtttcagtttccggcagaatcgatggcttcacggc | |
| gaaggatgatagatggaaaactttcgtctcctcaggttacatgtgtattc | |
| gatgctaacacgtgtctgagatatattagcttgatccaatcttcttctct | |
| aaatgtaatgttgggccagttggacttaaaatagtctctgtaaaccgttt | |
| tatgttgttgggcttttgctcgttcgttgtatgttatgttcatcttaata | |
| aaatcgtcacttgtg | |
| Complement of TTG3 | |
| SEQ ID NO:80 | |
| CACAAGTGACGATTTTATTAAGATGAACATAACATACAACGAACGAGCAA | |
| AAGCCCAACAACATAAAACGGTTTACAGAGACTATTTTAAGTCCAACTGG | |
| CCCAACATTACATTTAGAGAAGAAGATTGGATCAAGCTAATATATCTCAG | |
| ACACGTGTTAGCATCGAATACACATGTAACCTGAGGAGACGAAAGTTTTC | |
| CATCTATCATCCTTCGCCGTGAAGCCATCGATTCTGCCGGAAACTGAAAC | |
| TAAGTCGAATGAAACTCGAAGATTAGCTTCATCTGAATTTTTCCAACAAC | |
| TACAAAACAGAAGTAAGGATCTAATACGGATCTATTAATCTCAAAAGAGA | |
| AAAAAGATCTTCGATTAACCACCTCTGTTACTTAGACCCAATCGAAGCTT | |
| GAATATAAGAGATACAGCTAAAGGCAATCATAATAAGCTGAGGAAAGAGC | |
| TTTGAATCTTCATCTCAAAAACGCAAACTGCCTAATTATTATCCGTAAAC | |
| CTTAAACTCAAAGGATAGGTTGGAATTAGATCTAATTAAGCAAGCTTGGA | |
| CTGAAAACCAACGCCATCACCAACACACATACCCAATGAATTCTTATCTA | |
| AAGACAATCAACGGTCGAAACGTAACCGTGATATAAACCAAAAGGAAAAG | |
| AAAACCGAACTACGCCGGATTAACACCGTGAGCTAGCCATCACCGCTGAA | |
| AGAGCCGATGATGCTAGACAAGGGCCGACCATGTACTGCTGTGGAAGCCA | |
| ATATATGTCAGACGAAAGCCGGATACTACACCGGACGGCAATTCAACCAT | |
| CACCGACGACATCAATTTACCGTCGTCGAAAATTTTGGTTTGATGTCAAT | |
| CTCTCTCTCTGAAAATTTTGAGAGAGAGAAGAGAGAGAGGGCCAGAGTCA | |
| TTTTAATTTTGTCTTTAAGAATaaaatgaatacaatagtaacagaaaaaa | |
| tataatagccgtgactcgataaacattttaagcaaaaactcttgtatgat | |
| tctaaaaagaataatgaatataggccactcaccatcattacaagtccact | |
| gtccaccacacatcaaagctaataggtgttgtgataggcagtggcggaag | |
| aaggttataagtttattgccataggggctcacgtgtctccaccggattgg | |
| taagtaaagaattt | |
| predicted ORF (AC018664) | |
| SEQ ID NO:3 | |
| atggtgagtggcctatattcattattctttttagaatcatacaagagttt | |
| ttgcttaaaatgtttatcgagtcacggctattatattttttctgttacta | |
| ttagagagaatcaaaccaaaattttcgacgacggtaaattgatgtcgtcg | |
| gtgatggttgaattgccgtccggtgtagtatccggctttcgtctgacata | |
| tattggcttccacagcagtacatgttgttggaaaaattcagatgaagcta | |
| atcttcgagtttcattcgacttagtttcagtttccggcagaatcgatggc | |
| ttcacggcgaaggatgatagatggaaaactttcgtctcctcaggttacat | |
| gtgtattcgatgctaa | |
| Predicted polypeptide sequence (AC018664) | |
| SEQ ID NO:4 | |
| MVSGLYSLFFLESYKSFCLKCLSSHGYYIFSVTIRENQTKIFDDGKLMSS | |
| VMVELPSGVVSGFRLTYIGFHSSTCCWKNSDEANLRVSFDLVSVSGRIDG | |
| FTAKDDRWKTFVSSGYMCIRC | |
| CLUSTALW Analysis | |
| SEQ ID NO:1 | AAATTCTTTACTTACCAATCCGGTGGAGACACGTGAGCCCCTATGGCAATAAACTTATAA |
| EST:T44742 | ------------------------------------------------------------ |
| AC008262 | ------------------------------------------------------------ |
| SEQ ID NO:1 | CCTTCTTCCGCCACTGCCTATCACAACACCTATTAGCTTTGATGTGTGGTGGACAGTGGA |
| EST:T44742 | ------------------------------------------------------------ |
| AC008262 | ------------------------------------------------------------ |
| SEQ ID NO:1 | CTTGTAATGATGGTGAGTGGCCTATATTCATTATTCTTTTTAGAATCATACAAGAGTTTT |
| EST:T44742 | ------------------------------------------------------------ |
| AC008262 | ---------ATGGTGAGTGGCCTATATTCATTATTCTTTTTAGAATCATACAAGAGTTTT |
| | zzzzzzzzz*************************************************** |
| SEQ ID NO:1 | TGCTTAAAATGTTTATCGAGTCACGGCTATTATATTTTTTCTGTTACTATTGTATTCATT |
| EST:T44742 | ------------------------------------------------------------ |
| AC008262 | TGCTTAAAATGTTTATCGAGTCACGGCTATTATATTTTTTCTGTTACTATT--------- |
| | *************************************************** |
| SEQ ID NO:1 | TTATTCTTAAAGACAAAATTAAAATGACTCTGGCCCTCTCTCTCTTCTCTCTCTCAAAAT |
| SEQ ID NO:7 | --ATTCTTAAAGACAAAATTAAAATGACTCTGGCCCTCTCTCTCTTCTCTCTCTCAAAAT |
| EST:T44742 | -----------------------ATGACTCTGGCCCTCTCTCTCTTCTCTCTCTCAAAAT |
| AC008262 | ------------------------------------------------------------ |
| EST:AV822679 | zGATTCTTAAAGACAAAATTAAAATGACTCTGGCCCTCTCTCTCTTCTCTCTCTCAAAAT |
| | z*********************************************************** |
| SEQ ID NO:1 | TTTCAGAGAGAGAGATTGACATCAAACCAAAATTTTCGACGACGGTAAATTGATGTCGTC |
| SEQ ID NO:7 | TTTCAGAGAGAGA-------ATCAAACCAAAATTTTCGACGACGGTAAATTGATGTCGTC |
| EST:T44742 | TTTCAGAGAGAGAGATTGACATCAAACCAAAATTTTCGACGACGGTAAATTGATGTCGTC |
| AC008262 | ------AGAGAGA-------ATCAAACCAAAATTTTCGACGACGGTAAATTGATGTCGTC |
| EST:AV822679 | TTTCAGAGAGAGA-------ATCAAACCAAAATTTTCGACGACGGTAAATTGATGTCGTC |
| | ************************************************************ |
| SEQ ID NO:1 | GGTGATGGTTGAATTGCCGTCCGGTGTAGTATCCGGCTTTCGTCTGACATATATTGGCTT |
| SEQ ID NO:7 | GGTGATGGTTGAATTGCCGTCCGGTGTAGTATCCGGCTTTCGTCTGACATATATTGGCTT |
| EST:T44742 | GGTGATGGTTGAATTGCCGTCCGGTGTAGTATCCGGCTTTCGTCTGACATATATTGGCTT |
| AC008262 | GGTGATGGTTGAATTGCCGTCCGGTGTAGTATCCGGCTTTCGTCTGACATATATTGGCTT |
| EST:AV822679 | GGTGATGGTTGAATTGCCGTCCGGTGTAGTATCCGGCTTTCGTCTGACATATATTGGCTT |
| | ************************************************************ |
| SEQ ID NO:1 | CCACAGCAGTACATGGTCGGCCCTTGTCTAGCATCATCGGCTCTTTCAGCGGTGATGGCT |
| SEQ ID NO:7 | CCACAGCAGTACATGGTCGGCCCTTGTCTAGCATCATCGGCTCTTTCAGCGGTGATGGCT |
| EST:T44742 | CCACAGCAGTACATGGTCGGCCCTTGTCTAGCATCATCGGCTCTTTCAGCGGTGATGGCT |
| AC008262 | CCACAGCAGTACATG--------------------------------------------- |
| EST:AV822679 | CCACAGCAGTACATGGTCGGCCCTTGTCTAGCATCATCGGCTCTTTCAGCGGTGATGGCT |
| | ************************************************************ |
| SEQ ID NO:1 | AGCTCACGGTGTTAATCCGGCGTAGTTCGGTTTTCTTTTCCTTTT--GGTTTATATCACG |
| SEQ ID NO:7 | AGCTCACGGTGTTAATCCGGCGTAGTTCGGTTTTCTTTTCCTTTT--GGTTTATATCACG |
| EST:T44742 | AGCTCACGGTGTTAATCCGGCGTAGTTCGGTTTTNTTTTCCTTTTTGGGTTTATATCACG |
| AC008262 | ------------------------------------------------------------ |
| EST:AV822679 | AGCTCACGGTGTTAATCCGGCGTAGTTCGGTTTTCTTTTCCTTTT--GGTTTATATCACG |
| EST:CB260172 | ------------------------------------TTTCCTTTT--GGTTTATATCACG |
| | *********************************0************************** |
| SEQ ID NO:1 | G-TTACGTTTCGACC-GTTGATT-GTCTTTAGATAAGAA---TTCATTGGG-----TATG |
| SEQ ID NO:7 | G-TTACGTTTCGACC-GTTGATT-GTCTTTAGATAAGAA---TTCATTGGG-----TATG |
| EST:T44742 | GGTTACGTTTCGACCCGTTGATTTGTNTTTNAGNNAAANANTTNNATTNGGGGAATNTTG |
| AC008262 | ------------------------------------------------------------ |
| EST:AV822679 | G-TTACGTTTCGACC-GTTGATT-GTCTTTAGATCAGAA---TTCATTGGG-----TATG |
| EST:CB260172 | G-TTACGTTTCGACC-GTTGATT-GTCTTTAGATAAGAA---TTCATTGGG-----TATG |
| | * ************* ******* **0***00000*0*0zzz*00***0**zzzzz00** |
| SEQ ID NO:1 | TGTGTTGGTGATGGCG-TTGGTTTTCAG-TCCAAGCTTGCTTAATTAGATCTAATTCCAA |
| SEQ ID NO:7 | TGTGTTGGTGATGGCG-TTGGTTTTCAG-TCCAAGCTTGCTTAATTAGATCTAATTCCAA |
| EST:T44742 | TTTTTGGGAAAAGGNGGTTGGTTTTNANGCCCAAAGTT---------------------- |
| AC008262 | ------------------------------------------------------------ |
| EST:AA728500 | ------------------------------GCCAGCT-GCTNAATTNA-CCAAATNCCA- |
| EST:AV822679 | TGTGTTGGTGATGGCG-TTGGTTTTCAG-TCCAAGCTTGCTTAATTAGATCTAATTCCAA |
| EST:CB260172 | TGTGTTGGTGATGGCG-TTGGTTTTCAG-TCCAAGCTTGCTTAATTAGATCTAATTCCAA |
| | *0*0*0**00*0**0* ********0*0 00*0*00*****0****00*0*0***0*** |
| SEQ ID NO:1 | CCTATCCTTTGAGTTTAAGGTTTACGGATAATAATTAGGCAGTTTGCGTTTTTGAGATGAA- |
| SEQ ID NO:7 | CCTATCCTTTGAGTTTAAGGTTTACGGATAATAATTAGGCAGTTTGCGTTTTTGAGATGAA- |
| AC008262 | ------------------------------------------------------------ |
| EST:AA728500 | CCCNCCCNTGGGGTTAAGGGTTTCCGGNNAAAANTTAGGCNGTTTNCGTTTT-GAGATGAAG |
| EST:AV822679 | CCTATCCTTTGAGTTTAAGGTTTACGGATAATAATTAGGCAGTTTGCGTTTTTGAGATGAA- |
| EST:CB260172 | CCTATCCTTTGAGTTTAAGGTTTACGGATAATAATTAGGCAGTTTGCGTTTTTGAGATGAA- |
| | **000**0*0*0*****0***00**0*0******0****0********************** |
| SEQ ID NO:1 | GATTCAAAGCTCTTT-CCTCAGCTTATTATGATTGCCTTTAGCTG-TATCTCTTATATTCAAG |
| SEQ ID NO:7 | GATTCAAAGCTCTTT-CCTCAGCTTATTATGATTGCCTTTAGCTG-TATCTCTTATATTCAAG |
| AC008262 | zzz------------------------------------------------------------ |
| EST:AA728500 | GATNCAAAGCCCTTTCCCNCAGCTTATTAAGGATCCCNTTAGCTGGAACCCCTTATATTCAAG |
| EST:H76582 | zzz----------------------------------------------CTCTTATATTCAAG |
| EST:AV822679 | GATTCAAAGCTCTTT CCTCAGCTTATTATGATTGCCTTTAGCTG TATCTCTTATATTCAAG |
| EST:CB260172 | GATTCAAAGCTCTTT CCTCAGCTTATTATGATTGCCTTTAGCTG TATCTCTTATATTCAAG |
| | ***0******0**** **0**********0*00*0**0******* 0*0*0************ |
| SEQ ID NO:1 | CTTCGATTGGGTCTAA-GTAACAGAGGTGGTTAA-TCGAAGATCTTTTTTCTCTTTTGAG |
| SEQ ID NO:7 | CTTCGATTGGGTCTAA-GTAACAGAGGTGGTTAA-TCGAAGATCTTTTTTCTCTTTTGAG |
| AC008262 | ------------------------------------------------------------ |
| EST:AA728500 | CTCCGATTGGGTCTANGGTACCAGAGGTGGTTAAACCGAAGACCTTTTTNCNCTTTTGAG |
| EST:H76582 | CTTCGATNGGGTCTAA-GTAACAGAGGTGGTTAA-TCGAAGATCTNTNTTCTCTTTTGAG |
| EST:AV822679 | CTTCGATTGGGTCTAA-GTAACAGAGGTGGTTAA-TCGAAGATCTTTTTTCTCTTTTGAG |
| EST:CB260172 | CTTCGATTGGGTCTAA-GTAACAGAGGTGGTTAA-TCGAAGATCTTTTTTCTCTTTTGAG |
| | **0****0*******0 ***0************* 0******0**0*0*0*0******** |
| SEQ ID NO:1 | ATTAATAGATCCGTATTAGATCCTTACTTCTGTTTTGTAGTTGTTGGAAAAATTCAGATG |
| SEQ ID NO:7 | ATTAATAGATCCGTATTAGATCCTTACTTCTGTTTTGTAGTTGTTGGAAAAATTCAGATG |
| AC008262 | ----------------------------------------TTGTTGGAAAAATTCAGATG |
| EST:AA728500 | ATTAATAGANCCGTATTAGATCCTTACTCCTGTTTTGTAGTTGTTGGAAAAATTCNGATG |
| EST:H76582 | ATTAATAGATCCGTATTAGATCCTTACTTCTGNTNTGNAGTTGTTGGAAAAATTCAGATG |
| EST:AV822679 | ATTAATAGATCCGTATTAGATCCTTACTTCTGTTTTGTAGTTGNTGGAAAAATTCAGATG |
| EST:CB260172 | ATTAATAGATCCGTATTAGATCCTTACTTCTGTTTTGTAGTTGTTGGAAAAATTCAGATG |
| | *********0******************0***0*0**0*****0***********0**** |
| SEQ ID NO:1 | AAGCTAATCTTCGAGTTTCATTCGACTTAGTTTCAGTTTCCGGCAGAATCGATGGCTTCA |
| SEQ ID NO:7 | AAGCTAATCTTCGAGTTTCATTCGACTTAGTTTCAGTTTCCGGCAGAATCGATGGCTTCA |
| AC008262 | AAGCTAATCTTCGAGTTTCATTCGACTTAGTTTCAGTTTCCGGCAGAATCGATGGCTTCA |
| EST:AA728500 | AAGCTAATCTTCGAGTTTCATTCGACTTAGTTTCAGTTTCCGGCAGAATCGATGGCTTCA |
| EST:H76582 | AAGCTAATCTTCGAGTTTCATTCGACTTAGTNTCAGTTTCCGGAGAAATCGATGGCTTCA |
| EST:CB260172 | AAGCTAATCTTCGAGTTTCATTCGACTTAGTTTCAGTTTCCGGCAGAATCGATGGCTTCA |
| EST:AV822679 | AAGCTAATCTTCGAGTTTCAT--------------------------------------- |
| | *******************************0**************************** |
| SEQ ID NO:1 | CGGCGAAGGATGATAGATGGAAAACTTTCGTCTCCTCAGGTTACATGTGTATTCGATGCT |
| SEQ ID NO:7 | CGGCGAAGGATGATAGATGGAAAACTTTCGTCTCCTCAGGTTACATGTGTATTCGATGCT |
| AC008262 | CGGCGAAGGATGATAGATGGAAAACTTTCGTCTCCTCAGGTTACATGTGTATTCGATGCT |
| EST:AA728500 | CGGCGAAGGATGATAGATGGAAAACTTTCGTCTCCTCAGGTTACATGTGTATTCGATGCT |
| EST:H76582 | CGGCGANGGATGATAGATGGAAAACTTTCGTCTCCTCAGGTTACATGTGTATTCGATGCT |
| EST:CB260172 | CGGCGAAGGATGATAGATGGAAAACTTTCGTCTCCTCAGGTTACATGTGTATTCGATGCT |
| | ******0***************************************************** |
| SEQ ID NO:1 | AA---------------------------------------------------------- |
| SEQ ID NO:7 | AACACGTGTCTGAGATATATTAGCTTGATCCAATCTTCTTCTCTAAATGTAATGTTGGGC |
| AC008262 | AA---------------------------------------------------------- |
| EST:AA728500 | AACACGTGTCTGAGATATATTAGCTTGATCCAATCTTCTTCTCTAAA-TGTAATGTTGGG |
| EST:H76582 | AACACGTGTCTGAGATATATTAGCTTGATCCAATCTTCTTCTCTAAAATGTAANGTTGGG |
| EST:CB260172 | AACACGTGTCTGACATATATTAGCTTGATCCAATCTTCTTCTCTAAA-TGTAATGTTGGG |
| | *************0********************************* *****0****** |
| SEQ ID NO:1 | ------------------------------------------------------------ |
| SEQ ID NO:7 | CAGTTGGACTTAAAATAGTCTCTGTAAACCGTTTTATGTTGTTGGGCTTTTGCTCGTTCG |
| EST:AA728500 | CCAGTTGGACTTAAAATA-GTCTCTGTAAACCGTTTT-ATGTT-GTTGGGCTTTTGCTCG |
| EST:H76582 | CCAGTTGGACTTAAAATAAGTCTCTGTAAACCGTTTTTATGNCCGTTGGGCTTTTGCTCG |
| EST:CB260172 | CCAGTTGGACTTAAAATA-GTCTCTGTAAACCGTTTT-ATGTT-GTTGGGCTTTTGCTCG |
| | ****************** ****************** ***00 **************** |
| SEQ ID NO:1 | ------------------------------------------------------------ |
| SEQ ID NO:7 | TTGTATGTTAT-----GTTCATCTTAAT--AAAATCGTCACTTGTG |
| EST:AA728500 | TTCG-TTGTATGTTATGTTCATCTTAAT--AAAATCGTCACTTGTG-------------- |
| EST:H76582 | TTCGGNTGAATGTAATGTTCATCTTAAATAAANACCGTCACTTGGNGGAAAAAANTTNNC |
| EST:CB260172 | TTCG-TTGTATGTTATGTTCATCTTAAT--AAAATCGTCACTTGTGAAAAAAAAAA |
| | **** ***0****0*************0 **0*0**********0 |
| SEQ ID NO:1 | --- |
| SEQ ID NO:7 | |
| EST:AA728500 | --- |
| EST:H76582 | GNG |

‘*’ represents an identical nt position
‘0” represents a highly conserved nt position

### Example 5

### Gene Expression

Northern analysis was performed on the ttg3 mutant to assess the expression levels of the TTG3 gene, SEQ ID NO: 1, SEQ ID NO:2, SEQ ID NO:6 or SEQ ID NO:7 and the TTG1 gene. TTG1 is a gene that is known to be involved in transparent testa and glabrous phenotypes (Walker et al., 1999). The TTG1 gene sequence was identified in the TAIR accession number AT5G24520 and Genbank accession number NC 003076.

Total RNA was isolated from young leaf tissues of ttg3 plants and wild-type Arabidopsis var. Ws by use of the Qiagen RNAeasy Plant Mini Kit. Total RNA (10 μg) was separated by electrophoresis on a formaldehyde-1% agrose gel. Blotting was performed using Hybond N+ membrane according to manufacturer's instruction (Amersham Life Science). Blots were probed with ³²P-labeled DNA probes for the TTG3 or TTG1 genes. Blots were further probed using a probe against the constitutively expressed β-tubulin gene as a loading control, as described in Brandstatter and Kieber (1998).

The 882 bp TTG3 probe was produced by PCR amplification using primers identified by SEQ ID NO:24 and SEQ ID NO:25. The 1026 bp TTG1 probe was produced by PCR amplification using primers identified by SEQ ID NO:27 and SEQ ID NO:28. The 500 bp β-tubulin probe was produced by RT-PCR amplification using primers identified by SEQ ID NO:29 and SEQ ID NO:30.

Probes were prepared using the Prime-a-Gene system (Promega) and α-32P-dCTP. Hybridization was carried out using ExpressHyb system (Clontech) and a Phosphor imager was used to expose and quantify the blots.

RNA expression of TTG3 in fully expanded leaves of ttg3 mutants was reduced to 33% of the wild-type plants. The result suggests that, like TTG1, TTG3 is involved in tricome development, tannin and anthocyanidin production. It is likely that TTG1 and TTG3 are both components of a common pathway.

RNA expression of TTG1 in fully expanded leaves of ttg3 mutants was reduced to 51% of the wild-type plants. The down-regulation of TTG1 in ttg3 mutants suggests that TTG3 may act as an up-stream of TTG1 and is a positive regulator of TTG1 expression.

RNA expression of TTG1, TTG2 and TTG3 in fully expanded leaves of a ttg1 mutant was determined by Northern blot analysis. TTG2 and TTG3 expression are not altered in the ttg1 mutant. This is the expected result assuming TTG3 acts upstream of TTG1 and that TTG3 may act as a positive regulator of TTG1 expression.

Total RNA was isolated from developing leaf, mature leaf, stem and root, flower bud, open flower, and developing silique tissue from both ttg3 plants and wild-type Arabidopsis var. Ws by use of the Qiagen RNAeasy Plant Mini Kit. Northern analysis of the above tissues with TTG3 and TTG1 probes was performed to determine tissue specific expression patterns. TTG3 and TTG1 were expressed in all tissues tested from both ttg3 and wild-type plants. The expression levels in the ttg3 tissues were reduced relative to the wild-type.

Expression of TTG3 gene expression in wild type plants under cold stress and water stress were examined. Arabidopsis (Columbia) plants, four replicates per time point, were grown to first bolting, tendays, under 120 μE of light with a regime of 16 hour light and 8 hour dark, at 22° C. On day 10 optimal samples were harvested (whole shoot of the seedlings) into liquid N (time 0). Plants were moved to a growth chamber at 4° C. for a time period of 1 hour and 3 hours. The fully-expanded 5^{th} leaves were collected. Northern analysis showed that TTG3 expression was induced by 73% and 77% after 1 and 3 hours cold treatment relative to pretreatment expression levels. The TTG3 promoter is inducible by cold stress.

Arabidopsis (Columbia) plants, four replicates per time point, were grown as above. Plants were subjected to four days of drought stress by cessation of watering. Plants were sampled (whole shoots of the seedlings) on days 2, 3 and 4 after the onset of water stress treatment. Wilting of plants was observed on day 4. Northern analysis showed that TTG3 expression was induced by 21% after 2 and 329% after 3 days of drought treatment relative to pretreatment expression levels. The TTG3 expression at day 4 was 47% relative to pretreatment expression levels. The TTG3 promoter is inducible by drought stress.

### Example 6

### Anthocyanin Study

Plants were grown at 22° C., 16 hour photoperiod and ˜200 ummol/m2/s. Twelve replicates of the ttg3 mutant, a Ws control and the parent line were transplanted from agar plates into soil after 7 days on plates, grown under optimal conditions for an additional 3 days and then exposed to 4° C. for 5 days. Additional seedlings of the mutant and controls were also maintained on plates (0.7% agar, 0.5×MS, 1% sucrose+vitamins) and also exposed to the same cold treatment. After the cold treatment, none of the mutant plants, grown either in pots or on plates, displayed any visual signs of anthocyanin production along leaf petioles. The controls, however, had considerable anthocyanin accumulation, primarily at the apical meristem and along leaf petioles. Extraction of anthocyanin pigments from cold treated plants showed that ttg3 possessed only 5% of the anthocyanin content of the Ws control and 6% relative to the parent line, thereby correlating with the visual observations and indicative of a block in anthocyanin production.

Growth of ttg3 and control plants under optimal conditions showed that seedlings of the ttg3 mutant had an anthocyanin content of 39%, relative to the control plant. Following a cold treatment of 6 days (4° C.) there was a 2 fold stimulation of anthocyanin in ttg3 and in Ws wild type a 4.4 fold stimulation relative to pre-cold treatment anthocyanin levels. Comparison of ttg3 to wild type showed ttg3 had an anthocyanin content of only 19% relative to wild type.

### Example 7

### Physiology of the ttg3 Mutant

Plants were grown at 22° C., 16 hour photoperiod and ˜200 ummol/m2/s. Eight replicates of the ttg3 mutant, a Ws control and the parent line were grown under optimal conditions. Growth analysis was performed at three developmental stages and consisted of examination of a number of developmentally relevant parameters. At the vegetative stage, leaf number and shoot fresh weight was recorded. At the bolting stage, days to first flower, leaf number, plant height and shoot fresh weight were recorded. At the flowering stage days to first flower, leaf number, plant height, number of main stems, number of branches, number of pods, leaf fresh weight and stem fresh weight was recorded. No significant differences were observed between the ttg3 mutant and the parent line while only slight differences were observed relative to the wild-type controls.

### Example 8

### Drought Tolerance in the ttg3 Mutant

Drought studies with the Arabidopsis ttg3 mutant and wild type controls showed ttg3 had a slightly reduced shoot biomass higher soil water content during the drought treatment, higher shoot fresh to dry weight ratio (turgidity), and the water loss relative to shoot biomass tended to be reduced. These data are indicative of a drought tolerant phenotype. Examination of stomata density on mature leaves and stems, indicated that leaf stomata density was significantly lower on the upper surface of the leaf (67% of wild type). On the lower leaf surface the difference was not significant; however, the trend was for reduced stomatal density (84% of wild type). No differences in stomata density were found on the main stems.

Plants were grown in a replicated water-stress experiment. All pots were filled with equal amounts of homogeneous premixed and wetted soil. Growth conditions were 16 hour daylight (150-200 μmol/m²/s) at 22° C. and 70% relative humidity. On the day that the first flower opened drought treatment was initiated first by equalizing the soil water content in each pot on a weight basis and then by cessation of watering. Pots were weighed daily for to determine daily water loss. At the end of the water stress treatment plants were harvested for shoot biomass determination. At the end of the experiment the dry weight of the pots was determined and this allowed calculation of soil water content as a percentage of initial. Water loss relative to final shoot biomass was calculated.

Alternatively, at the end of the water stress treatment plants are re-watered and allowed to complete the life cycle and determination of biomass and yield data obtained. Physiological parameters are assessed under stressed and optimal conditions. For example, shoot and root biomass accumulation, soil water content, and water loss alone or as a function of parameters such as biomass, seed yield, leaf number and leaf area.

### Example 9

### Seedling Growth, Plate Study

Seeds were plated (8 seeds/plate) on 0.7% agar supplemented with 1% sucrose, 0.5×MS and vitamins. There were 8 replicates for the ttg3 mutant, a Ws control and the parent line. Plates were grown vertically at 22° C., 16 hour photoperiod and ˜200 ummol/m2/s. Photographs were taken on days 4 and 7 and seedlings were harvested after 10 days of growth. Image analysis (Image J) was used to obtain actual root lengths.

There were no significant differences in seedling biomass between the ttg3 mutant and controls. Examination of the 5 day old roots showed that the ttg3 mutant has longer roots and significantly lower root hair density than the wild type.

### Example 10

### Arabidopsis ttg3 Seed Analysis

Plants were grown at 22° C., 16 hour photoperiod and ˜200 ummol/m²/s. Ten replicates of the ttg3 mutant, two Ws wild-type controls and the parent line were grown under optimal conditions. Seeds were then harvested and weighed. Results of four experiments indicated there was no significant difference in seed yield between the ttg3 mutant and the parent line.

Seed composition analysis was performed as follows. Two replicates for each entry, ttg3 mutant, parent line and Ws control, were analyzed for fatty acid composition, total oil total protein and acid fiber. Each replicate contained ˜2 g of seed. Seeds were analyzed by POS Pilot Plant Corp (Analytical Services, 118 Veterinary Rd, Saskatoon, Saskatchewan, S7N 2R4)

Oil analysis results are presented in Table 2, which shows that the ttg3 mutant seeds have increased oil, 114% relative to the parent and 120% relative to the Ws control, and reduced acid fiber, 60% relative to the parent line and 61% relative to the Ws control. Protein content was not affected (107% relative to the parent line and 96% relative to the Ws control). Fatty acid composition was similar for the mutant and each control, as shown in Table 3.

A second POS pilot seed analysis was done on two samples of ttg3 and two samples of wild type Arabidopsis, ecotype Ws. Protein, oil and acid digestible fiber contents are presented in Table 4 and fatty acid composition shown in Table 5. The results of the second analysis confirmed the first analysis.

Together these analysis show that the ttg3 mutant has increased seed oil and decreased seed fiber contents while protein levels remain largely unchanged. Moreover, the seed oil profiles are equivalent to that of wild type. Seed size was assessed by determining 100 seed weights, no significant differences were found

TABLE 2
Protein, Oil, and Fiber analysis 1.

| | Protein (%) | Oil (%) | Acid Fiber (%) |
|---|---|---|---|
| Mutant | 24.6 | 45.9 | 5.11 |
| 57 | 23.0 | 40.2 | 8.49 |
| Ws | 25.6 | 38.2 | 8.36 |

TABLE 3
Fatty acid composition 1.

| | C14 | C16 | C16:1 | C18 | C18:1 | C18:2 | C18:3n3 | C20 |
|---|---|---|---|---|---|---|---|---|
| Mutant | 0.07 | 6.38 | 0.24 | 3.15 | 18.06 | 28.34 | 15.38 | 2.19 |
| Ws | 0.08 | 6.44 | 0.27 | 3.07 | 17.6 | 28.7 | 15.1 | 2.18 |
| 57 | 0.07 | 6.46 | 0.26 | 3.13 | 16.69 | 28.15 | 15.95 | 2.26 |
| | C20:1 | C20:2 | C20:3n3 | C22 | C22:1n9 | C22:2 | C24 | C24:1 |
| Mutant | 21.23 | 1.94 | 0.38 | 0.26 | 1.84 | 0.04 | 0.09 | 0.13 |
| Ws | 21.6 | 1.88 | 0.37 | 0.28 | 1.88 | 0.05 | 0.1 | 0.13 |
| 57 | 21.77 | 2.02 | 0.41 | 0.28 | 1.95 | 0.05 | 0.09 | 0.14 |

TABLE 4
Protein, Oil, and Fiber analysis 2.

| | Protein (%) | Oil (%) | Acid Fiber (%) |
|---|---|---|---|
| ttg3 Mutant | 19.6 (99% of Ws) | 52 (118% of Ws) | 5.3 (60% of Ws) |
| Ws | 19.7 | 44 | 8.9 |

TABLE 5
Fatty acid composition 2.

| | C14 | C16 | C16:1 | C18 | C18:1 | C18:2 | C18:3n3 | C20 |
|---|---|---|---|---|---|---|---|---|
| Mutant | 0.06 | 6.45 | 0.27 | 3.47 | 17.23 | 27.8 | 14.98 | 2.31 |
| Ws | 0.07 | 6.47 | 0.28 | 3.39 | 15.69 | 27.52 | 15.91 | 2.38 |
| | C20:1 | C20:2 | C20:3n3 | C22 | C22:1n11 | C22:1n9 | C24 | C24:1 |
| Mutant | 19.42 | 1.93 | | 0.28 | 1.80 | 0.04 | 0.11 | 0.14 |
| Ws | 20.03 | 1.99 | | 0.29 | 1.77 | 0.05 | 0.11 | 0.14 |

### Example 11

### Construct Generation

The TTG3 gene can be manipulated to provide over expression or down regulation in a plant cell. For over expression a TTG3 sequence is provided in a sense orientation functionally adjacent to a promoter sequence. The promoter sequence may provide for constitutive expression such as 35S CaMv promoter, or be a promoter having specific expression characteristics, such as a seed promoter (napin, Ban). Down-regulation can be achieved using known molecular techniques, for example, antisense technology or hair-pin technology. The particular method is one of choice provided that the result is reduced expression of a TTG3 sequence.

Suitible promotor useful in preparing nucleic acid constructs for the over an unde expression of TTG3 include the following:

HPR Promoter

SEQ ID NO:9

gaagcagcagaagccttgatcatcttcctttgtctcaacctgaaactctt
ttttttctttcattgtttgttctcttttcactgtggatgtagataattgt
ttttaatgaaatgaagaaatattgatttgccttttgacataattttgtta
ataatcttgattacaaattttagtcagtgtttgatgcatagttgcatact
gcagagttgagtttggatatggccacgtcagcattatctcgttaccaaaa
cgtaaggtccaaactcagataatacaaacgaagcagttctttgtcactct
atcatcaacatatgaaccacaccaaaaaagaacaaaatcgtagataatga
tcatgcaaaaccgaccgttggatcttactttcgatttcaaaccacataaa
tcttagtgactgagctaaaaaactgaaattttttaaaaggcaagacctcc
tctgtttccatattctcaccacagaagaactcttgaggctttctcttttc
tctaccatggcg
BAN Promoter

SEQ ID NO:10

attgcttaaggccagattctgtgaaacatggacaagaacagagcaagtta
tgttgaattgactcgtgtaattcgtgaaacagaacatagcaagtccaagt
tgtgttaaaaactgcagagaatttgacagattggtggaagtaaaaagcat
tcttttgcaactcattttaagatcggcaaagaaaaaattgaagtaacaga
accttactgtaacactattcgttactctaaagctgtgttatattgtttag
agacagaaataatcaaactcttgtgataatttggtagatgataacaaatc
agaactctgaaggtcaatcttttttgattcttaggtgaagacaagttggt
tatttcaaagatcacgtgcttaccttctaaaacagccttattgatctact
gttgtacctaatgagcaaggactatttgcaaatctttttacttcttatat
agaagtctcaagacgataaactcataacaactaaatctctatctctgtaa
tttcaaaagtacaatcatggac
SC Promoter

SEQ ID NO:11

aagcttaaattgacattatatatgaaagacaataaataagatacaacgtt
ttatataaacaatatggattatatacaatagttaaaaaatccttagatgt
ctgttaaacacagcgttttggtgagttaactcgtatggtataagtttaaa
gcattccagtaactatattggtatcaaaatctacattcatattaattcaa
ataattttttacgtttaccaaaataaatccatataaacatgcgattgatc
ttgggcaagagaggtgttaaaatagtatcttggtatgactattagttgaa
atttgactaatgcattcaagaatgtatatttattgtaagaaatgctcacg
tatcttatttgataaagaatgctcacgtaccttaataaatacgtgtgcat
agagtttgtgtgtatatatagatggttatgatcagggaagaagaacacaa
cacaactcacctcaaacagacaatttaattccaaaagataaaaacaatca
aaagagatct
Napin Promoter

SEQ ID NO:12

cttttaaaccaacttagtaaacgtttttttttttaattttatgaagttaa
gtttttaccttgtttttaaaaagaatcgttcataagatgccatgccagaa
cattagctacacgttacacatagcatgcagccgcggagaattgtttttct
tcgccacttgtcactcccttcaaacacctaagagcttctctctcacagca
cacacatacaatcacatgcgtgcatgcattattacacgtgatcgccatgc
aaatctcctttatagcctataaattaactcatccgcttcactctttactc
aaaccaaaactcatcaatacaatgtagattaaaaacatacacg

Exemplary constructs useful in modulating TTG3 expression in plants are decribed below:

P_{35S}-TTG3:

A portion of SEQ ID NO:1 predicted to include the TTG3 transcribed sequence was cloned into pBI121ΔGUS, a modified pBI121 vector lacking the GUS gene, downstream of the 35S promoter (P_{35S}-TTG3).

The P_{35S}-TTG3 construct was produced as follows. A 874 bp region of SEQ ID NO:1 was PCR amplified using the primer pair identified as SEQ ID NO:24 and SEQ ID NO:25. The resulting fragment was digested with XbaI and SacI, and inserted into pBI121ΔGUS digested with XbaI and SacI.

P_{TTG3}-TTG3

TTG3 was cloned into pBI101 such that the TTG3 gene was expressed by its endogenous promoter (P_{TTG3}-TTG3). The P_{TTG3}-TTG3 construct was produced as follows. A 1972 bp region was PCR amplified using genomic DNA as template and the primer pair identified as SEQ ID NO:26 and SEQ ID NO:25. The resulting fragment was digested with XbaI and SacI, and inserted into pBI101 digested with XbaI and SacI.

P_{35S}-TTG3-B

The P_{35S}-TTG3-B construct was produced as follows. A 915 bp region was RT-PCR amplified using the primer pair identified as SEQ ID NO:34 and SEQ ID NO:35 from ecotype Columbia total RNA and therefore lacks the 7 nt insertion “GATTGAC” found in ecotypes Ws and Ler. The resulting fragment was digested with XbaI and BamHI, and inserted into pBI121ΔGUS digested with XbaI and BamI. The TTG3-B sequence, represented by SEQ ID NO:7 represents the predicted transcribed region based on EST sequence analysis.

P_{HPR}-TTG3

The promoter region of P_{35S}-TTG3 was removed by digestion with HindIII and BamHI and replaced with a PCR amplified HPR promoter. The HPR promoter was obtained by PCR amplification using the primer pair identified as SEQ ID NO:53 and SEQ ID NO:54.

P_{35S}-Antisense-TTG3

Construction of the TTG3 antisense vector is done as described herein. The TTG3 fragment was produced using PCR amplification of SEQ ID NO:1. Template for the PCR reaction was genomic DNA. Appropriate primers were those identified as SEQ ID NO:32 and SEQ ID NO:33. The PCR generated fragment was digested with BamHI and XbaI and cloned into a modified pBI121 vector (pBI121ΔGUS) digested with BamHI and XbaI. The GUS sequence had been removed from pBI121 by digestion with SmaI and EcolCRI and the vector ligated after purification of the vector from the GUS insert to produce the pBI121-ΔGUS vector. The insertion of the TTG3 BamHI-XbaI fragment results in the construct P_{35S}-antisense-TTG3.

P_{5C}-HP-TTG3

The pBI121 vector was digested with SmaI and SacI, the GUS sequence and the vector fragments were purified from one another. The isolated GUS fragment was digested using EcoRV and the 1079 bp. blunt ended EcoRV/SacI fragment isolated. This was ligated back into the digested parent vector at the SmaI/SacI sites. This intermediate vector (pBI121tGUS) is used in the subsequent production of the hairpin vectors. The seedcoat (SC) promoter was obtained by PCR amplification using the primer pair identified as SEQ ID NO:49 and SEQ ID NO:50. The PCR product was digested with HindIII and XbaI and cloned into the pBI121tGUS vector replacing the HindIII and XbaI 35S promoter fragment. The TTG3 fragment to be used as the gene specific hairpin sequence is isolated by PCR. Primers identified as SEQ ID NO:32 and SEQ ID NO:33 were used to generate an antisense fragment of 874 bp in length and the primers identified as SEQ ID NO:46 and SEQ ID NO:25 were used to generate an sense fragment of 874 bp in length. Cloning of the sense orientation fragment was achieved by digesting the PCR TTG3 fragment with SacI and ligation into the SacI site at the 3′ end of GUS. To insert the antisense fragment upsteam of GUS, a BamHI and XbaI digest was performed and the PCR amplified TTG3 fragment digested with BamHI and XbaI was ligated into the vector to yield the final construct, P_{35S}-HP-TTG3.

P_{35S}-HP-TTG3-2

The P_{35S}-HP-TTG3-2 construct was produced using a portion of the TTG3 gene rather than the full TTG3 gene as follows. A 725 bp region was RT-PCR amplified from total RNA using the primer pair identified as SEQ ID NO:38 and SEQ ID NO:39 for cloning as the antisense orientation fragment. The amplified product is digested with XbaI and BamHI and cloned into pBI121tGUS as the antisense TTG3 sequence. A 417 bp region was PCR amplified from total RNA using the primer pair identified as SEQ ID NO:40 and SEQ ID NO:41 for cloning as the sense orientation fragment. The amplified product is digested with SacI and cloned as the sense TTG3 sequence, thereby resulting in the P_{35S}-HP-TTG3-2 vector construct.

P_{35S}-HP-TTG3-3

The P_{35S}-HP-TTG3-3 construct was produced using a portion of the TTG3 gene rather than the full TTG3 gene as follows. A 417 bp region was RT-PCR amplified from total RNA using the primer pair identified as SEQ ID NO:42 and SEQ ID NO:43 for cloning as the antisense orientation fragment. The amplified product is digested with BamHI and XbaI and cloned into pBI121tGUS as the antisense TTG3 sequence. A 417 bp region was PCR amplified from total RNA using the primer pair identified as SEQ ID NO:44 and SEQ ID NO:45 for cloning as the sense orientation fragment. The amplified product is digested with SacI and cloned as the sense TTG3 sequence, thereby resulting in the P_{35S}-HP-TTG3-3 vector construct.

P_{BAN}-HP-TTG3

The P_{BAN}-HP-TTG3 construct was derived from P_{SC}-HP-TTG3 by the substitution of the BANYLUS gene promoter for that of the seed coat promoter. The BAN promoter was obtained by PCR amplification using the primer pair identified as SEQ ID NO:47 and SEQ ID NO:48. The PCR product was digested with HindIII and XbaI and replaced the 35S promoter of pBI121. The BAN promoter was then re-excised from the vector by ApaI and XhaI digestion and the resulting fragment replaced the seed coat promoter of the P_{SC}-HP-TTG3 construct similarly digested thereby producing the P_{BAN}-HP-TTG3 construct

P_{35S}-HP-TTG3

The P₃₅S-HP-TTG3 construct was derived from P_{SC}-HP-TTG3 by the substitution of the CaMV 35S gene promoter for that of the promoter Seed Coat (SC). The 35S promoter was obtained by ApaI and XbaI digestion of the pBI121 vector and cloned so as to replace the SC promoter of the P_{SC}-HP-TTG3 construct.

P_{NAPIN}-HP-TTG3

The P_{NAPIN}-HP-TTG3 construct was derived from P_{35S}-HP-TTG3 by the substitution of the napin gene promoter for that of the CaMV 35S promoter. The napin promoter was obtained by PCR amplification using the primer pair identified as SEQ ID NO:51 and SEQ ID NO:52. The PCR product was digested with HindIII and XbaI and replaced the 35S promoter of pBI121. The Napin promoter was then re-excised from the vector by ApaI and XbaI digestion and the resulting fragment replaced the seed coat promoter of the P_{SC}-HP-TTG3 construct similarly digested thereby producing the P_{Napin}-HP-TTG3 construct

pGAD-TTG3-FL

The pEGAD-AE binary vector, derived from pEGAD by removal of the GFP sequence, was double digested with SmaI and BamHI. A 915 bp TTG3 gene fragment was amplified by PCR using the primer pair identified as SEQ ID NO:61 and SEQ ID NO:62. The PCR product was cut by SmaI and BamHI double digestion, and then ligated into previously digested pEGAD-AE vector.

pGAD-T5-D200

pEGAD-AE binary vector, derived from pEGAD by removal of the GFP sequence, was double digested with SmaI and BamHI. A 718 bp TTG3 gene fragment was amplified by PCR using the primer pair identified as SEQ ID NO:63 and SEQ ID NO:62. The resulted PCR fragment covers TTG3 sequence from 198 to 916 nucleotide position. The PCR product was cut by SmaI and BamHI double digestion, and then ligated into previously digested pEGAD-AE vector.

pGAD-T5-D400

pEGAD-AE binary vector, derived from pEGAD by removal of the GFP sequence, was double digested with SmaI and BamHI. A 517 bp TTG3 gene fragment was amplified by PCR using the primer pair identified as SEQ ID NO:64 and SEQ ID NO:62. The resulted PCR fragment covers TTG3 sequence from 400 to 916 nucleotide position. The PCR product was cut by SmaI and BamHI double digestion, and then ligated into previously digested pEGAD-AE vector.

pGAD-T5-D600

pEGAD-AE binary vector, derived from pEGAD by removal of the GFP sequence, was double digested with SmaI and BamHI. A 319 bp TTG3 gene fragment was amplified by PCR using the primer pair identified as SEQ ID NO:65 and SEQ ID NO:62. The resulted PCR fragment covers TTG3 sequence from 598 to 916 nucleotide position. The PCR product was cut by SmaI and BamHI double digestion, and then ligated into previously digested

pGAD-T5-D800

pEGAD-AE binary vector, derived from pEGAD by removal of the GFP sequence, was double digested with SmaI and BamHI. A 120 bp TTG3 gene fragment was amplified by PCR using the primer pair identified as SEQ ID NO:66 and SEQ ID NO:62. The resulted PCR fragment covers TTG3 sequence from 797 to 916 nucleotide position. The PCR product was cut by SmaI and BamHI double digestion, and then ligated into previously digested

pGAD-T3-D200

pEGAD-AE binary vector, derived from pEGAD by removal of the GFP sequence, was double digested with SmaI and BamHI. A 717 bp TTG3 gene fragment was amplified by PCR using the primer pair identified as SEQ ID NO:61 and SEQ ID NO:67. The resulted PCR fragment covers TTG3 sequence from 1 to 718 nucleotide position. The PCR product was cut by SmaI and BamHI double digestion, and then ligated into previously digested

pGAD-T3-D400

pEGAD-AE binary vector, derived from pEGAD by removal of the GFP sequence, was double digested with SmaI and BamHI. A 520 bp TTG3 gene fragment was amplified by PCR using the primer pair identified as SEQ ID NO:61 and SEQ ID NO:68. The resulted PCR fragment covers TTG3 sequence from 1 to 521 nucleotide position. The PCR product was cut by SmaI and BamHI double digestion, and then ligated into previously digested

pGAD-T3-D600

pEGAD-AE binary vector, derived from pEGAD by removal of the GFP sequence, was double digested with SmaI and BamHI. A 319 bp TTG3 gene fragment was amplified by PCR using the primer pair identified as SEQ ID NO:61 and SEQ ID NO:69. The resulted PCR fragment covers TTG3 sequence from 1 to 320 nucleotide position. The PCR product was cut by SmaI and BamHI double digestion, and then ligated into previously digested

pGAD-T3-D800

pEGAD-AE binary vector, derived from pEGAD by removal of the GFP sequence, was double digested with SmaI and BamHI. A 123 bp TTG3 gene fragment was amplified by PCR using the primer pair identified as SEQ ID NO:61 and SEQ ID NO:70. The resulted PCR fragment covers TTG3 sequence from 1 to 124 nucleotide position. The PCR product was cut by SmaI and BamHI double digestion, and then ligated into previously digested

### Example 12

### Rescue of an Arabidopsis ttg3 Mutant by a Wild-Type TTG3 Gene

Rescue of the ttg3 phenotype by SEQ ID NO:6 or SEQ ID NO:7 expression was performed in order to confirm the relationship between the gene and the phenotype. Constructs were produced to demonstrate that SEQ ID NO:6 or SEQ ID NO:7 was capable of complementing the ttg3 mutant by restoration of a wild-type phenotype. Constructs were made for transformation into the ttg3 mutant. Constructs were introduced into Arabidopsis ttg3 mutant plants by the method of dipping flowering plants into an Agrobacterium culture transformed with the desired vector construct based on the method of Andrew Bent in Clough S J and Bent A F, 1998.

T1 seeds from the transformed plants were harvested and the seeds examined visually. The T1 seeds produced by the ttg3 plants transformed with P_{35S}-TTG3, P_{35S}-TTG3-B and ttg3:P_{TTG3}-TTG3, were a mixture of brown seeds and seeds with brown patches representing a rescue event or yellow seeds representing untransformed seeds. The T1 seeds were germinated on plates and grown for physiology, segregation, genetic and molecular analysis. The T1 plants developing from brown T1 seed, a rescue event, produced normal leaf trichomes. RNA was isolated from leaf tissue for Northern analysis and RNA expression levels of TTG3 that were equivalent to wild type levels. The levels of TTG1 expression in the ttg3 mutant transformed with P_{35S}-TTG3 was assessed by Northern alalysis and shown to be restored to wild-type levels.

### Example 13

### Plants Having Increased Expression of TTG3

Wild type Arabidopsis plants were transformed with the P_{35S}-TTG3, P_{35S}-TTG3-B or the P_{TTG3}-TTG3 construct by the Arabidopsis standard transformation methods. Normal trichome development was observed in all T1 lines.

The 35S-TTG3 construct was transformed into Arabidopsis and eleven homozygous transgenic events were produced in ecotype Columbia (Col.) and twenty-one homozygous events were produced in Ws background. From the eleven Columbia events six showed increased expression of TTG3 relative to Columbia wild-type, ranging from 2 fold to 8.6 fold increase. From the twenty-one Ws events 16 showed increased expression relative to Ws wild type, ranging from 0.5 fold to 38 fold increase. Seed composition analysis was done, as described in Example 20, on all the lines and found that all 11 events in Columbia background showed reduced lipid (oil) content, by as much as a 27% reduction and a corresponding increased fiber content, as much as 15.5% over wild type levels. Over-expression of TTG3 in wild-type does not increase the level of TTG1 expression above that of wild-type, as determined by Northern analysis.

Physiological assessment of the 35S-TTG3 lines in Arabidopsis ecotypes Ws and Columbia had the following characteristics.

None of the 35S-TTG3 transgenic lines showed any differences in trichomes on the first set of true leaves, mature rosette leaves, stem leaves, stems, flowers, sepals and no trichomes were found on cotyledons. No differences were found in optimal seed yield between transgenic lines and controls.

Cold stress was applied as described in Example 6 to 35S-TTG3 transgenic lines in both Ws and Col. ecotypes, with the exception that the stress period was for 6 days. No significant differences were observed in plant shoot biomass before or after cold stress. However, a few transgenic lines showed slightly lower inhibition of growth by cold stress relative to controls. The anthocyanin content was measured and a trend identified for marginally higher levels of anthocyanin after cold stress in the two ecotypes relative to controls. Anthocyanin content was expressed as a percentage of optimal, transgenic lines showed a 2.4 fold greater stimulation of anthocyanin production.

Stem cross-sections were taken of the main stem 2 cm. above soil level at the time of first open flower from 35S-TTG3 transgenic lines and the ttg3 mutant. The number of vascular bundles, stem diameter, thickness of the cortex layer and thickness of the fiber layer were measured using a Lica stereoscope. The ttg3 mutant line appeared to have a reduced layer of fiber than wild type controls. The 35S-TTG3 transgenic lines appeared to have an increased thickness of the stem fiber layer.

Plants may be produced that have increased expression of the TTG3 gene by a variety of methods. For example, a promoter tagging approach may be used. A promoter sequence lacking an operably linked 3′ gene is inserted into a plant genome such that expression of a gene located 3′ to the insertion site may be expressed. In such a way, a plant may be produced in which a heterologous promoter becomes situated upstream of the TTG3 gene and directs expression of the gene.

Plants may be produced that have increased expression of the TTG3 gene by a variety of methods. For example, a promoter tagging approach may be used. A promoter sequence lacking an operably linked 3′ gene is inserted into a plant genome such that expression of a gene located 3′ to the insertion site may be expressed. In such a way, a plant may be produced in which a heterologous promoter becomes situated upstream of the TTG3 gene and directs expression of the gene. Alternatively, a gene construct having at least a portion of a TTG3 sequence in a sense orientation such as P_{35S}-TTG3 or P_{TTG3}-TTG3, described above, can be transformed into a plant and transgenic plants over-expressing the TTG3 gene may be identified by molecular and phenotypic characterization.

### Example 14

### TTG3 Promoter Analysis

The TTG3 promoter identified as SEQ ID NO:5 or SEQ ID NO:8 was operatively linked to a GUS gene in the vector pBI101. The promoter was obtained by PCR amplification using the primer pair identified by SEQ ID NO:26 and SEQ ID NO:31. The resulting PCR product was digested with XbaI and SmaI, and inserted into pBI101 digested with XbaI and SmaI wherein the promoter sequence would provide for transcription of the adjacent GUS gene. The constructs were introduced into wild-type Arabidopsis plants by the flower dipping method as described above. T1 seeds from the transformed plants are harvested for analysis of GUS staining activity in the various tissue of the T1 plants. Subsequent generations can likewise be analyzed for promoter activity.

A series of 5′ TTG3 promoter deletions were made to assess the promoter characteristics. The series was comprised of five constructs all having a common 3′ end that included the first 49 bp of translated sequence. For naming of the constructs the site of the T-DNA insertion in the ttg3 mutant was used as the base reference point. The series comprised promoter sequences of constructs of 1264 bp (P-TTG3), 1002 bp (P-700), 706 bp (P-400), 482 bp (P-200) and 290 bp (P-TDNA). Promoter sequences were obtained by PCR amplification from Arabidopsis genomic DNA using the following primer pairs. P-TTG3 was amplified using the primier pair identified by SEQ ID NO:26 and SEQ ID NO:60. P-700 was amplified using the primier pair identified by SEQ ID NO:56 and SEQ ID NO:60. P-400 was amplified using the primier pair identified by SEQ ID NO:57 and SEQ ID NO:60. P-200 was amplified using the primier pair identified by SEQ ID NO:58 and SEQ ID NO:60. P-TDNA was amplified using the primier pair identified by SEQ ID NO:59 and SEQ ID NO:60. The resulting PCR products were digested-with XbaI and SmaI, and inserted into pBI101 digested with XbaI and SmaI wherein promoter sequences would provide for transcription of the adjacent GUS gene. The constructs were introduced into wild-type Arabidopsis plants by the flower dipping method as described above. T1 seeds from the transformed plants are harvested for analysis of GUS staining activity in the various tissue of the T1 plants. Subsequent generations can likewise be analyzed for promoter activity.

Plants were grown and tissue harvested at appropriate developmental stages; for example, young leaf, mature leaf, stem and root, flower bud, open flower, developing silique tissue, seed or any such desired tissue. Plant tissue was added to 1 mL GUS staining solution (50 mM NaPO₄, pH 7.0, 0.1% Triton 3-100, 1 mM EDTA, 2 mM DTT, 0.5 mg/mL 3-GlcA) and incubated overnight at 37° C. The staining solution was replaced with 1 mL fixation buffer (10% formaldehyde, 50% ethanol) and incubated for 30 minutes at room temperature. The fixation buffer was replaced with 80% ethanol and incubated for 1 hour at room temperature. The 80% ethanol was replaced with 100% ethanol and incubated for 1 hour at room temperature. The tissue was assessed for blue staining, indicating GUS activity.

Analysis of P-TTG3 activity was examined in leaf tissue of one week old seedlings and found to possess activity at the growing apex, the expanding leaf tip, clusters of cells at developing leaf veins, [at the base of trichomes] and in the hypocotyl vascular tissue. In mature leaves expression was observed in clustered foci about the leaf margin. Analysis of seed tissue is performed. Analysis of the P-700 promoter showed no differences from the expression characteristics obtained using the P-TTG3 promoter. Analysis of the P-200 promoter determined expression to be present in young leaf tips. Analysis of the P-TDNA promoter showed no detectable expression.

A TTG3 promoter sequence requires additional sequence than the 290 bp of the P-TDNA promoter and that the P-200 sequence provides limited activity while the P-700 sequence is sufficient for full activity.

Predicted TTG3 Promoter

SEQ ID NO:5

gcgaatacgtaaacgtatgacctggtttgttcggtttgcgagttaaagtc
aatttgaagaaaaagagtaatttaagataagctaatatgaaatctgatct
aatgtggcatttcaatattgtaatatcttgcttgataaagagaaaacaaa
taatgcgaatgcggcatctctctctcttgtctccacgtcaacgattttgc
tagatttgattccaatcccttttccagcttacctaccttttccctcaaaa
aattgtgttaaatatggcttcatctcttaagaaatactttccattttgga
agtaatttcctaatttcttagcaaggaaaaaaacctaaaaaggaaatgac
tataacattttataatgtaaaaggattaaaaaaaagggtagaatatcata
attatcatttttacattgttaagaaacttatttatgtatccatggttact
tttgtttattttttataattaaaacaaaaaaagtaattccttttaattta
tcattaaattcgaaagtataaaaagcgttggaccacaatccattctgaga
aaaaaccccatcctatgaaatttaactcataaaagtgtcaaaatgttctt
gaaaattcaaaatttatcatgaataaaactttaaatatttctactcacta
ttcttattttttagtatatatatctatatattatatttagaatccttcac
aaaaataattgttgcataatactcattggttttacacgttttctgggctc
aagttttgttccaatttttttgatatatgaaaattctaaggtctataaac
atattggatgcaatgaaaaggtctaaataaacaaaatcacaatctatctt
cacaacaatttcttagaaattcaaacttcacataagttgtttgaatcctt
ttttaattaaaaggatatattttaatatgagttaactaaaataattagac
acttaaaccaaattaaaagtaataaattctttacttaccaatccggtgga
gacacgtgagcccctatggcaataaacttataaccttcttccgccactgc
ctatcacaacacctattagctttgatgtgtggtggacagtggacttgtaa
tg
Predicted TTG3-B Promoter

SEQ ID NO:8

gcgaatacgtaaacgtatgacctggtttgttcggtttgcgagttaaagtc
aatttgaagaaaaagagtaatttaagataagctaatatgaaatctgatct
aatgtggcatttcaatattgtaatatcttgcttgataaagagaaaacaaa
taatgcgaatgcggcatctctctctcttgtctccacgtcaacgattttgc
tagatttgattccaatcccttttccagcttacctaccttttccctcaaaa
aattgtgttaaatatggcttcatctcttaagaaatactttccattttgga
agtaatttcctaatttcttagcaaggaaaaaaacctaaaaaggaaatgac
tataacattttataatgtaaaaggattaaaaaaaagggtagaatatcata
attatcatttttacattgttaagaaacttatttatgtatccatggttact
tttgtttattttttataattaaaacaaaaaaagtaattccttttaattta
tcattaaattcgaaagtataaaaagcgttggaccacaatccattctgaga
aaaaaccccatcctatgaaatttaactcataaaagtgtcaaaatgttctt
gaaaattcaaaatttatcatgaataaaactttaaatatttctactcacta
ttcttattttttagtatatatatctatatattatatttagaatccttcac
aaaaataattgttgcataatactcattggttttacacgttttctgggctc
aagttttgttccaatttttttgatatatgaaaattctaaggtctataaac
atattggatgcaatgaaaaggtctaaataaacaaaatcacaatctatctt
cacaacaatttcttagaaattcaaacttcacataagttgtttgaatcctt
ttttaattaaaaggatatattttaatatgagttaactaaaataattagac
acttaaaccaaattaaaagtaataaattctttacttaccaatccggtgga
gacacgtgagcccctatggcaataaacttataaccttcttccgccactgc
ctatcacaacacctattagctttgatgtgtggtggacagtggacttgtaa
tgatggtgagtggcctatattcattattctttttagaatcatacaagagt
ttttgcttaaaatgtttatcgagtcacggctattatattttttctgttac
tattgtattcatttt
P-TTG3

SEQ ID NO:13

gcgaatacgtaaacgtatgacctggtttgttcggtttgcgagttaaagtc
aatttgaagaaaaagagtaatttaagataagctaatatgaaatctgatct
aatgtggcatttcaatattgtaatatcttgcttgataaagagaaaacaaa
taatgcgaatgcggcatctctctctcttgtctccacgtcaacgattttgc
tagatttgattccaatcccttttccagcttacctaccttttccctcaaaa
aattgtgttaaatatggcttcatctcttaagaaatactttccattttgga
agtaatttcctaatttcttagcaaggaaaaaaacctaaaaaggaaatgac
tataacattttataatgtaaaaggattaaaaaaaagggtagaatatcata
attatcatttttacattgttaagaaacttatttatgtatccatggttact
tttgtttattttttataattaaaacaaaaaaagtaattccttttaattta
tcattaaattcgaaagtataaaaagcgttggaccacaatccattctgaga
aaaaaccccatcctatgaaatttaactcataaaagtgtcaaaatgttctt
gaaaattcaaaatttatcatgaataaaactttaaatatttctactcacta
ttcttattttttagtatatatatctatatattatatttagaatccttcac
aaaaataattgttgcataatactcattggttttacacgttttctgggctc
aagttttgttccaatttttttgatatatgaaaattctaaggtctataaac
atattggatgcaatgaaaaggtctaaataaacaaaatcacaatctatctt
cacaacaatttcttagaaattcaaacttcacataagttgtttgaatcctt
ttttaattaaaaggatatattttaatatgagttaactaaaataattagac
acttaaaccaaattaaaagtaataaattctttacttaccaatccggtgga
gacacgtgagcccctatggcaataaacttataaccttcttccgccactgc
ctatcacaacacctattagctttgatgtgtggtggacagtggacttgtaa
tgatggtgagtggcctatattcattattctttttagaatcatacaagagt
ttttgcttaaaatgtttatcgagtcacggctattatattttttctgttac
tattgtattcattttATTCTTAAAGACAAAATTAAAATGACTCTGGCCCT
CTCTCTCTTCTCTCT
P700

SEQ ID NO:14

tatggcttcatctcttaagaaatactttccattttggaagtaatttccta
atttcttagcaaggaaaaaaacctaaaaaggaaatgactataacatttta
taatgtaaaaggattaaaaaaaagggtagaatatcataattatcattttt
acattgttaagaaacttatttatgtatccatggttacttttgtttatttt
ttataattaaaacaaaaaaagtaattccttttaatttatcattaaattcg
aaagtataaaaagcgttggaccacaatccattctgagaaaaaaccccatc
ctatgaaatttaactcataaaagtgtcaaaatgttcttgaaaattcaaaa
tttatcatgaataaaactttaaatatttctactcactattcttatttttt
agtatatatatctatatattatatttagaatccttcacaaaaataattgt
tgcataatactcattggttttacacgttttctgggctcaagttttgttcc
aatttttttgatatatgaaaattctaaggtctataaacatattggatgca
atgaaaaggtctaaataaacaaaatcacaatctatcttcacaacaatttc
ttagaaattcaaacttcacataagttgtttgaatccttttttaattaaaa
ggatatattttaatatgagttaactaaaataattagacacttaaaccaaa
ttaaaagtaataaattctttacttaccaatccggtggagacacgtgagcc
cctatggcaataaacttataaccttcttccgccactgcctatcacaacac
ctattagctttgatgtgtggtggacagtggacttgtaatgatggtgagtg
gcctatattcattattctttttagaatcatacaagagtttttgcttaaaa
tgtttatcgagtcacggctattatattttttctgttactattgtattcat
tttATTCTTAAAGACAAAATTAAAATGACTCTGGCCCTCTCTCTCTTCTC
TCT
p400

SEQ ID NO:15

catcctatgaaatttaactcataaaagtgtcaaaatgttcttgaaaattc
aaaatttatcatgaataaaactttaaatatttctactcactattcttatt
ttttagtatatatatctatatattatatttagaatccttcacaaaaataa
ttgttgcataatactcattggttttacacgttttctgggctcaagttttg
ttccaatttttttgatatatgaaaattctaaggtctataaacatattgga
tgcaatgaaaaggtctaaataaacaaaatcacaatctatcttcacaacaa
tttcttagaaattcaaacttcacataagttgtttgaatccttttttaatt
aaaaggatatattttaatatgagttaactaaaataattagacacttaaac
caaattaaaagtaataaattctttacttaccaatccggtggagacacgtg
agcccctatggcaataaacttataaccttcttccgccactgcctatcaca
acacctattagctttgatgtgtggtggacagtggacttgtaatgatggtg
agtggcctatattcattattctttttagaatcatacaagagtttttgctt
aaaatgtttatcgagtcacggctattatattttttctgttactattgtat
tcattttATTCTTAAAGACAAAATTAAAATGACTCTGGCCCTCTCTCTCT
TCTCTCT
P200

SEQ ID NO:16

attctaaggtctataaacatattggatgcaatgaaaaggtctaaataaac
aaaatcacaatctatcttcacaacaatttcttagaaattcaaacttcaca
taagttgtttgaatccttttttaattaaaaggatatattttaatatgagt
taactaaaataattagacacttaaaccaaattaaaagtaataaattcttt
acttaccaatccggtggagacacgtgagcccctatggcaataaacttata
accttcttccgccactgcctatcacaacacctattagctttgatgtgtgg
tggacagtggacttgtaatgatggtgagtggcctatattcattattcttt
ttagaatcatacaagagtttttgcttaaaatgtttatcgagtcacggcta
ttatattttttctgttactattgtattcattttATTCTTAAAGACAAAAT
TAAAATGACTCTGGCCCTCTCTCTCTTCTCTCT
P-TDNA

SEQ ID NO:17

aattctttacttaccaatccggtggagacacgtgagcccctatggcaata
aacttataaccttcttccgccactgcctatcacaacacctattagctttg
atgtgtggtggacagtggacttgtaatgatggtgagtggcctatattcat
tattctttttagaatcatacaagagtttttgcttaaaatgtttatcgagt
cacggctattatattttttctgttactattgtattcattttATTCTTAAA
GACAAAATTAAAATGACTCTGGCCCTCTCTCTCTTCTCTCT

### Example 15

### Isolation of TTG3 Homologues from Plants

Isolation of a cDNA or genomic sequence corresponding to the TTG3 gene contained in the genomic sequence identified by SEQ ID NO:2 from a desired plant species can be accomplished by RT-PCR techniques and appropriate primer pairs. Alternatively, appropriate primers can be used to amplify from genomic DNA. The plant may be an Arabidopsis, Brassica, corn, soybean, sunflower, cotton or any other desired plant species.

Total RNA was isolated from young leaf tissue of Arabidopsis using a Qiagen RNAeasy Plant Mini Kit. Reverse transcription of TTG3 was performed using 5 μg of RNA reverse transcriptase (Invitrogen Supescript Reverse Transcriptase II, RNaseH) and a TTG3 specific primer identified by SEQ ID NO:25. Subsequently PCR amplification of the RT-PCR products was performed using the primer pair identified by SEQ ID NO:34 and SEQ ID NO:25. DNA fragments were cloned into a suitable vector, such as pBluescript TA, and subjected to sequence analysis. Alternatively one can design primers that amplify a portion of the transcribed sequence, such as with the primer pair identified by SEQ ID NO:38 and SEQ ID NO:39 or SEQ ID NO:42 and SEQ ID NO:43.

Other methods are available to identify a TTG3 homologue and are apparent to one of skill in the art. For example functional cloning, homology searches across highly related species, gene walking or RNA profiling can be used to obtain a TTG3 gene from a desired plant species.

Functional cloning may be accomplished by construction of a genomic library in a vector suitable for transformation into plants. Transformation of the library into an Arabidopsis ttg3 mutant line is done according to the standard protocols. Transformants are screened for lines in which at least a ttg3 phenotype has been rescued, that of wild type. The sequence introduced by the genomic library is identified and further analysis, either bioinformatics or continued functional mapping identifies the TTG3 sequence from the species from which the genomic library was made. The observation that the Arabidopsis TTG3 gene is functional in Brassica, based on the modification of oil and fiber profiles, suggests that although there may be little sequence homology between species, the gene is functionally related.

Homology searches across related species can be used to identify a TTG3 sequence in a step wise fashion. Analysis of a number of species and sub-species between two species of choice, i.e. Arabidopsis to Brassica, is done in a phylogentetic step wise manner to preserve homology between each step. Similarly, one may use genetic markers or genes that are linked to Arabidopsis TTG3 to identify regions from other plants of interest which are likely to contain the TTG3 gene.

### Example 16

### Isolation of Genomic TTG3 Sequence from Plant Species

Genomic DNA is isolated from plant tissue using Qiagen DNeasy Plant Mini Kit. PCR is used to amplify the sequence of interest using appropriately designed primer pairs. For example, PCR is performed using primers identified by SEQ ID NO:24 and SEQ ID NO:25. Alternatively, primers identified by SEQ ID NO:26 and SEQ ID NO:25 may be used. Other primer pairs will be obvious to the skilled practitioner. Degenerate nucleotides can be incorporated into the primers which can be used such that specificity constraints are relaxed, allowing for greater variability in sequence specificity. A DNA fragment so produced, is isolated and cloned into a vector, such as pBluescript by TA cloning for example. The cloned fragment is sequenced to confirm the identity of the cloned fragment.

### Example 17

### Production and Isolation of Plants Having Mutations in TTG3

Mutagens such as fast neutron irradiation, chemical mutagens such as EMS, T-DNA insertional inactivation in the gene or its regulatory sequences may be used to produce gene mutations, interruptions or inhibited expression in a TTG3 gene. The desired mutant is screened and isolated based on selection of the TTG3 phenotype. Genetic and molecular analysis is performed to confirm the nature of the mutation.

Alternatively, the process known as gene tilling (McCallum, et al. 2000) can be used to isolate a suitable mutant of SEQ ID NO: 1, SEQ ID NO:2, SEQ ID NO:6 or SEQ ID NO:7 and having the TTG3 phenotype.

### Example 18

### Down-Regulation of TTG3 in Plants

Plants can be produced having the desirable traits of a ttg3 mutant by the down-regulation of SEQ ID NO:1 SEQ ID NO:2, SEQ ID NO:6, or SEQ ID NO:7. Down-regulation can be achieved via a variety of mechanisms such as, but not limited to, mutant generation, antisense technology, co-suppression, dominant-negative expression RNAi technology or other RNA transcript mediated regulation mechanisms such as those involving non-coding-RNA transcripts. Transformation of a desired species can be done using a variety of methods known in the art and one applicable to the particular species being transformed can be chosen.

For example SEQ ID NO:1 SEQ ID NO:2, SEQ ID NO:6, or SEQ ID NO:7 or a portion thereof, is cloned in an antisense orientation downstream of a promoter operable in plants, for example 35SCaMV promoter, in a vector such as pBI121 or pEGAD. The construct is transformed via Agrobacterium into Brassica, corn, cotton, sunflower or soybean. The transformed cell is regenerated into a transformed plant having the ttg3 traits and phenotypes. Transformed plants are screened for those having the desired characteristics.

Construction of the TTG3 antisense vector is done as described herein and in Example 11. The TTG3 fragment is produced using PCR amplification of SEQ ID NO:1 SEQ ID NO:2, SEQ ID NO:6, or SEQ ID NO:7. Template for the PCR reaction is genomic DNA or RNA if the primers are within the transcribed region, specifically SEQ ID NO:7. Other sources of template can be used such as the cDNA transcribed from the TTG3 gene. Appropriate primers are those identified as SEQ ID NO:34 and SEQ ID NO:35. The PCR generated fragment is digested with BamHI and XbaI and cloned into a modified pBI121 vector (pBI121ΔGUS) digested with BamHI and XbaI. The GUS sequence had been removed from pBI121 by digestion with SmaI and Eco1CRI and the vector ligated after purification of the vector from the GUS insert to produce the pBI121-ΔGUS vector. The insertion of a TTG3 BamHI-XbaI fragment results in the construct P_{35S}-antisense-TTG3-B.

Down-regulation of genes can be achieved by hairpin (HP) constructs. A fragment is cloned in both the sense and antisense orientation, which are separated by a spacer sequence. A spacer sequence can be any sequence capable of being transcribed and does not terminate transcription. Transcription produces a RNA transcript in which the sense and antisense portions are complimentary and anneal to form a dsRNA molecular species that through post transcriptional gene silencing mechanisms recognize and down-regulate both the transgene product and the endogenous gene. Construction of a P_{35S}-HP-TTG3 construct was done as described below and in Example 11. Generally, the cloning strategy may involve truncating the GUS gene of pBI121 and flanking the GUS sequence with a TTG3 gene sequence fragment in the antisense orientation upstream of the GUS and, in the sense orientation, on the downstream side of GUS. The TTG3 gene sequence may be the entire gene sequence or a portion thereof. The portion thereof is preferably at least 50 nucleotides in length and more preferably at least 20 nucleotides in length. The GUS sequence represents a spacer and can be replaced with other suitable sequences as desired, so long as it permits transcription to proceed through the spacer region. The pBI121 vector was digested with SmaI and SacI, the GUS sequence and the vector fragments were purified from one another. The isolated GUS fragment was digested using EcoRV and the 1079 bp. blunt ended EcoRV/SacI fragment isolated. This was ligated back into the digested parent vector at the SmaI/SacI sites. This intermediate vector is used in the subsequent production of the hairpin vectors. The TTG3 fragment to be used as the gene specific hairpin sequence is isolated by PCR. Primers identified as SEQ ID NO:32 and SEQ ID NO:33 were used to generate a fragment approximately 1 kb in length. Alternatively, the primer pairs identified as SEQ ID NO:34 and SEQ ID NO:35 may be used. Other hair pin constructs can be made as described in Experiment 11 using primer sets as described therein. Cloning of the sense orientation fragment was achieved by digesting the PCR TTG3 fragment with SacI and ligation into the SacI site at the 3′ end of GUS. To insert the same fragment upsteam of GUS, a BamHI and XbaI digest was performed and the PCR amplified TTG3 fragment digested with BamHI and XbaI was ligated into the vector to yield the final construct, P_{35S}-HP-TTG3.

Down-regulation of genes can be achieved via a co-suppression mechanism in which a DNA construct having a sense construct designed to over-express the gene results in the suppression of both transgene and homologous endogenous genes. Transgenic plants are screened for lines or individuals having the characteristics of a down-regulated TTG3. Over-expression constructs such as those described herein can be used to produce a co-suppression event.

The above vector constructs are modified to place the genes under the control of alternative promoters, such as, but not limited to, a promoter region that drives gene expression such as promoters from the following genes, TTG3, HPR, BAN, SC, Napin, RD29A or MuA. Alternatively cryptic promoters may be employed. This is accomplished by excising the 35S promoter sequence and replacing it with a promoter sequence of choice. Promoters may be constitutive, have tissue, developmental, temporal spatial specificity or possess other unique expression characteristics.

### Example 19

### TTG3 Acts Upstream of TTG1

RNA expression of TTG1 in fully expanded leaves of ttg3 mutants was reduced to 51% of the wild-type plants. Over-expression of TTG3 by the P_{35S}-TTG3 construct in the ttg3 mutant restores TTG1 expression to wild-type levels. Over-expression of TTG3 in wild-type does not increase the level of TTG1 expression above the normal wild-type level.

RNA expression of TTG1, TTG2 and TTG3 in fully expanded leaves of a ttg1 mutant was determined by Northern blot analysis. TTG2 and TTG3 expression were not altered in the ttg1 mutant. These are the expected result assuming TTG3 acts upstream of TTG1 and indicative that TTG3 may act as a positive regulator of TTG1 expression.

### Example 20

### Brassica Transformation

Transgenic Brassica napus plants were produced using constructs to result in over-expression or down-regulation of the TTG gene

Transgenic Brassica plants were produced using Agrobacterium mediated transformation of cotyledon petiole tissue. Seeds were sterilized as follows. Seeds were wetted with 95% ethanol for a short period of time such as 15 seconds. Approximately 30 ml of sterilizing solution I was added (70% Javex, 100 μl Tween20) and left for approximately 15 minutes. Solution I was removed and replaced with 30 ml of solution II (0.25% mecuric chloride, 100 μl Tween20) and incubated for about 10 minutes. Seeds were rinsed with at least 500 ml double distilled sterile water and stored in a sterile dish. Seeds were germinated on plates of ½ MS medium, pH 5.8, supplemented with 1% sucrose and 0.7% agar. Fully expanded cotyledons were harvested and placed on Medium I (Murashige minimal organics (MMO), 3% sucrose, 4.5 mg/L benzyl adenine (BA), 0.7% phytoagar, pH5.8). An Agrobacterium culture containing the nucleic acid construct of interest was grown for 2 days in AB Minimal media. The cotyledon explants were dipped such that only the cut portion of the petiole is contacted by the Agrobacterium solution. The explants were then embedded in Medium I and maintained for 5 days at 24° C., with 16, 8 hr light dark cycles. Explants were transferred to Medium II (Medium I, 300 mg/L timentin,) for a further 7 days and then to Medium III (Medium II, 20 mg/L kanamycin). Any root or shoot tissue which had developed at this time was dissected away. Transfer explants to fresh plates of Medium III after 14-21 days. When regenerated shoot tissue developed the regenerated tissue was transferred to Medium IV (MMO, 3% sucrose, 1.0% phytoagar, 300 mg/L timentin, 20 mg/L kanamycin). Once healthy shoot tissue developed shoot tissue dissected from any callus tissue was dipped in 10× IBA and transferred to Medium V (Murashige and Skooge (MS), 3% sucrose, 0.2 mg/L indole butyric acid (IBA), 0.7% agar, 300 mg/L timentin, 20 mg/L kanamycin) for rooting. Healthy plantlets were transferred to soil.

Transgenic soybean, corn and cotton can be produced using Agrobacterium-based methods which are known to one of skill in the art. Alternatively one can use a particle or non-particle biolistic bombardment transformation method. An example of non-particle biolistic transformation is given in U.S. patent application 20010026941. Viable plants are propagated and homozygous lines are generated. Plants are tested for the presence of drought tolerance, physiological and biochemical phenotypes as described elsewhere.

### Example 21

### Expression of an Arabidopsis TTG3 gene in Brassica napus

The constructs identified as P_{35S}-TTG3 and P_{HPR}-TTG3 and were transformed into Brassica napus and transgenic plants recovered and analyzed. The constructs identified as P_{TTG}-TTG3 and P_{35S}-TTG3-B are transformed into Brassica napus and transgenic plants recovered and analyzed.

P_{35S}-TTG3 transgenic plants were assessed to confirm the presence of the construct by PCR, Southern and Northern blot analysis. Transgenic plants were confirmed by PCR and Southern blot analysis. PCR was performed using the primer pair identified by SEQ ID NO:36 and SEQ ID NO:37. Analysis of T0 plants by Northern blot demonstrated that 13 of 16 lines expressed the Arabidopsis TTG3 gene. The Arabidopsis TTG3 gene does not hybridize with any homologous Brassica transcripts at the stringency used for the Northern (0.2×SSC, 65° C.). Transgenic plants were advanced to subsequent generations to isolate low copy number transgenic lines. Copy numbers ranged from 1 to 8.

P_{HPR}-TTG3 transgenic plants were assessed to confirm the presence of the construct by PCR, Southern and Northern blot analysis. Transgenic plants were confirmed by PCR and Southern blot analysis. PCR was performed using the primer pair identified by SEQ ID NO:55 and SEQ ID NO:37. Transgenic plants were advanced to subsequent generations to isolate low copy insert transgenic plants.

Analysis of T1 Seed

Transgenic T1 seed was harvested and analyzed for seed fiber and oil contents, based on the method of Bligh, E. G. and Dyer, W. J., A rapid method of total lipid extraction and purification in Canadian Journal of Biochemistry and Physiology, 1959 Vol. 37. pg 911-917.

Solvent was prepared by mixing 1 part of chloroform with 2 part of methanol and 0.8 part of water. Samples were prepared by addition of approximately 0.1 g of seed to a pre-weighed microfuge tube. The tube was weighed to determine the exact seed weight. Stainless steel beads were added and total tube weight determined. The sample was immersed on liquid nitrogen to freeze tissue. Seeds were ground using a Mixer Mill for a total of 2 minutes at 20 second intervals. Tissue was refrozen in liquid nitrogen and reground for a total of 2 minutes at 20 second intervals. Solvent was added, 0.2 ml and reground for 1 minute followed by an additional 0.4 ml solvent and another 30 seconds of grinding. An additional 0.2 ml of solvent was added and the sample mixed thoroughly by inversion of tube. Chloroform, 0.264 ml was added and mixed well. Water was added 0.264 ml and the sample again well mixed. Samples were centrifuged for 5 minutes at 13000×g to separate the organic and aqueous phases. The fiber component is located at the interface. The upper methanol phase was transferred to a pre-weighed tube and the lower chloroform phase to another pre-weighed tube. The fiber component was re-extracted by addition of 0.3 ml of solvent, ground for 1 minute, 0.1 ml of chloroform and 0.1 ml of water were added and mixing well after each addition. Samples were re-centrifuged for 5 minutes at 13000×g. Methanol and Chloroform layers were transferred and pooled with that collected from the first extraction. Samples were allowed to evaporate overnight and vacuum desiccated to ensure dryness. Tubes weights were determined. The insoluble fraction represents the fiber and protein components, the chloroform layer contained lipids and oils, and the methanol layer contained sugars.

Twenty one P_{35S}-TTG3 lines were examined for oil and fiber content. The seed was T1 seed produced from heterozygous T0 plants and compared to untransformed parental control lines. In all cases, in which oil and fiber content were altered from controls, the variations in oil and fiber content was inversely related. Of the nineteen lines tested thirteen had fiber levels whose means and standard deviation were greater than controls, ten of which also had corresponding reduced oil content. Fiber content of the transgenic lines ranged from 98% to 113% relative to controls while oil contents ranged from 107% to 72% of controls. There was a clear trend observed correlating the increased fiber and decreased oil contents. The six lines that did not show greater fiber contents were approximately equivalent to controls.

Lines having the desired oil and fiber content and are shown to be low copy number transgenic lines are advanced for detailed analysis.

### Example 22

### Reduced Expression of TTG3 in Brassica napus

Introduction of a gene construct to down-regulate the Brassica TTG3 gene is accomplished by a variety of molecular means. Introduction of an antisense, hair-pin or siRNA construct of TTG3 is used to down-regulate an endogenous TTG3 gene.

Constructs useful for down-regulation include P_{35S}-antisense-TTG3, P_{35S}-HP-TTG3, P_{35S}-HP-TTG3-2, P_{35S}-HP-TTG3-3, P_{BAN}-HP-TTG3, P_{SC}-HP-TTG3, P_{Napin}-HP-TTG3.

Using the above constructs, transgenic plants are produced and screened for a variety of characteristics. Among these are reduced expression of endogenous TTG3, increased seed oil and decreased fiber content, yellow seed color and reduced or eliminated trichome development. Other phenotypes may be apparent.

The P_{35S}-HP-TTG3, P_{BAN}-HP-TTG3, P_{Napin}-HP-TTG3 and P_{35S}-antisense-TTG3 constructs were transformed into Brassica napus. Transgenic lines were established and plants are assessed as above.

### Example 23

### Functional Analysis of the TTG3 Gene

A series of progressive TTG3 deletions from either the 5′ end or 3′ end of the TTG3 gene were generated for functional analysis of the gene. The constructs were generated from the pEGAD vector as described in Example 11. The series was comprised of the full length TTG3 gene (915 bp) and 4 deletions from each end resulting in TTG3 5′ deletion fragments of 718, 517, 319 and 120 nucleotides in length and TTG3 3′. deletion fragments of 717, 520, 319 and 123 nucleotides in length.

Constructs are transformed into ttg3 mutant plants and the efficacy of rescue by each construct is assessed as described in Example 12. Other physiological aspects are assessed according to the previous examples. The minimum requirements for gene functionality are thereby determined. Use of said minimum functionally required sequence can be made in designing over-expression or down-regulation constructs.

The following constructs were used to analyze the function of TTG3:

pGAD-TTG3 FL 915 bp

SEQ ID NO:71

attcttaaagacaaaattaaaatgactctggccctctctctcttctctct
ctcaaaattttcagagagagaatcaaaccaaaattttcgacgacggtaaa
ttgatgtcgtcggtgatggttgaattgccgtccggtgtagtatccggctt
tcgtctgacatatattggcttccacagcagtacatggtcggcccttgtct
agcatcatcggctctttcagcggtgatggctagctcacggtgttaatccg
gcgtagttcggttttcttttccttttggtttatatcacggttacgtttcg
accgttgattgtctttagatcagaattcattgggtatgtgtgttggtgat
ggcgttggttttcagtccaagcttgcttaattagatctaattccaaccta
tcctttgagtttaaggtttacggataataattaggcagtttgcgtttttg
agatgaagattcaaagctctttcctcagcttattatgattgcctttagct
gtatctcttatattcaagcttcgattgggtctaagtaacagaggtggtta
atcgaagatcttttttctcttttgagattaatagatccgtattagatcct
tacttctgttttgtagttgttggaaaaattcagatgaagctaatcttcga
gtttcattcgacttagtttcagtttccggcagaatcgatggcttcacggc
gaaggatgatagatggaaaactttcgtctcctcaggttacatgtgtattc
gatgctaacacgtgtctgagatatattagcttgatccaatcttcttctct
aaatgtaatgttgggccagttggacttaaaatagtctctgtaaaccgttt
tatgttgttgggcttttgctcgttcgttgtatgttatgttcatcttaata
aaatcgtcacttgtg
pGAD-T5-D200 718 bp

SEQ ID NO:72

tctagcatcatcggctctttcagcggtgatggctagctcacggtgttaat
ccggcgtagttcggttttcttttccttttggtttatatcacggttacgtt
tcgaccgttgattgtctttagatcagaattcattgggtatgtgtgttggt
gatggcgttggttttcagtccaagcttgcttaattagatctaattccaac
ctatcctttgagtttaaggtttacggataataattaggcagtttgcgttt
ttgagatgaagattcaaagctctttcctcagcttattatgattgccttta
gctgtatctcttatattcaagcttcgattgggtctaagtaacagaggtgg
ttaatcgaagatcttttttctcttttgagattaatagatccgtattagat
ccttacttctgttttgtagttgttggaaaaattcagatgaagctaatctt
cgagtttcattcgacttagtttcagtttccggcagaatcgatggcttcac
ggcgaaggatgatagatggaaaactttcgtctcctcaggttacatgtgta
ttcgatgctaacacgtgtctgagatatattagcttgatccaatcttcttc
tctaaatgtaatgttgggccagttggacttaaaatagtctctgtaaaccg
ttttatgttgttgggcttttgctcgttcgttgtatgttatgttcatctta
ataaaatcgtcacttgtg
pGAD-T5-D400 517 bp

SEQ ID NO:73

tatcctttgagtttaaggtttacggataataattaggcagtttgcgtttt
tgagatgaagattcaaagctctttcctcagcttattatgattgcctttag
ctgtatctcttatattcaagcttcgattgggtctaagtaacagaggtggt
taatcgaagatcttttttctcttttgagattaatagatccgtattagatc
cttacttctgttttgtagttgttggaaaaattcagatgaagctaatcttc
gagtttcattcgacttagtttcagtttccggcagaatcgatggcttcacg
gcgaaggatgatagatggaaaactttcgtctcctcaggttacatgtgtat
tcgatgctaacacgtgtctgagatatattagcttgatccaatcttcttct
ctaaatgtaatgttgggccagttggacttaaaatagtctctgcaaaccgt
tttatgttgttgggcttttgctcgttcgttgtatgttatgttcatcttaa
taaaatcgtcacttgtg
pGAD-T5-D600 319 bp

SEQ ID NO:74

tccTtacttctgttttgtagttgttggaaaaattcagatgaagctaatct
tcgagtttcattcGacttagtttcagtttccggcagaatcgatggcttca
cggcgaaggatgatagatggaaaActttcgtctcctcaggttacatgtgt
attcgatgctaacacgtgtctgagatatattagCttgatccaatcttctt
ctctaaatgtaatgttgggccagttggacttaaaatagtctctGtaaacc
gttttatgttgttgggcttttgctcgttcgttgtatgttatgttcatctt
aatAaaatcgtcacttgtg
pGAD-T5-D800

SEQ ID NO:75

tctctaaatgtaatgttgggccagttggacttaaaatagtctctGtaaac
cgttttatgttgttgggcttttgctcgttcgttgtatgttatgttcatct
taatAaaatcgtcacttgtg
pGAD-T3-D200 717 bp

SEQ ID NO:76

attcttaaagacaaaattaaaatgactctggccctctctctcttctctct
ctcaaaattttcagagagagaatcaaaccaaaattttcgacgacggtaaa
ttgatgtcgtcggtgatggttgaattgccgtccggtgtagtatccggctt
tcgtctgacatatattggcttccacagcagtacatggtcggcccttgtct
agcatcatcggctctttcagcggtgatggctagctcacggtgttaatccg
gcgtagttcggttttcttttccttttggtttatatcacggttacgtttcg
accgttgattgtctttagatcagaattcattgggtatgtgtgttggtgat
ggcgttggttttcagtccaagcttgcttaattagatctaattccaaccta
tcctttgagtttaaggtttacggataataattaggcagtttgcgtttttg
agatgaagattcaaagctctttcctcagcttattatgattgcctttagct
gtatctcttatattcaagcttcgattgggtctaagtaacagaggtggtta
atcgaagatcttttttctcttttgagattaatagatccgtattagatcct
tacttctgttttgtagttgttggaaaaattcagatgaagctaatcttcga
gtttcattcgacttagtttcagtttccggcagaatcgatggcttcacggc
gaaggatgatagatgga
pGAD-T5-D400 520 bp

SEQ ID NO:77

attcttaaagacaaaattaaaatgactctggccctctctctcttctctct
ctcaaaattttcagagagagaatcaaaccaaaattttcgacgacggtaaa
ttgatgtcgtcggtgatggttgaattgccgtccggtgtagtatccggctt
tcgtctgacatatattggcttccacagcagtacatggtcggcccttgtct
agcatcatcggctctttcagcggtgatggctagctcacggtgttaatccg
gcgtagttcggttttcttttccttttggtttatatcacggttacgtttcg
accgttgattgtctttagatcagaattcattgggtatgtgtgttggtgat
ggcgttggttttcagtccaagcttgcttaattagatctaattccaaccta
tcctttgagtttaaggtttacggataataattaggcagtttgcgtttttg
agatgaagattcaaagctctttcctcagcttattatgattgcctttagct
gtatctcttatattcaagct
pGAD-T5-D600 319 bp

SEQ ID NO:78

attcttaaagacaaaattaaaatgactctggccctctctctcttctctct
ctcaaaattttcagagagagaatcaaaccaaaattttcgacgacggtaaa
ttgatgtcgtcggtgatggttgaattgccgtccggtgtagtatccggctt
tcgtctgacatatattggcttccacagcagtacatggtcggcccttgtct
agcatcatcggctctttcagcggtgatggctagctcacggtgttaatccg
gcgtagttcggttttcttttccttttggtttatatcacggttacgtttcg
accgttgattgtctttaga
pGAD-T5-D800 123 bp

SEQ ID NO:79

attcttaaagacaaaattaaaatgactctggccctctctctcttctctct
ctcaaaattttcagagagagaatcaaaccaaaattttcgacgacggtaaa
ttgatgtcgtcggtgatggttga

### Example 24

### A Selectable Marker in Plant Species where a ttg3 Mutant is Available

The loss of the TTG3 phenotype by introduction of a functional TTG3 gene is an indicator of transformation, as detailed above in the case of Arabidopsis transformation with either P_{35S}-TTG3, P_{35S}-TTG3-B or P_{TTG3}-TTG3. Transformation of a ttg3 mutant line with a vector comprising a wild-type allele of SEQ ID NO: 1, SEQ ID NO:2, SEQ ID NO:6 or SEQ ID NO:7 or homologous TTG3 sequence, can result in a plant having a selectable phenotype, that being the loss of at least a TTG3 phenotype, such as the loss of the transparent testa phenotype. When in use as a selectable marker, a second gene, being a gene of interest, can be included on the vector comprising the TTG3 marker gene. Alternatively, the gene of interest and TTG3 marker gene may be on separate vectors.

### Example 25

### A Selectable Marker in Plant Species Where No ttg3 Mutant is Available

Where no ttg3 mutant is available, use of an antisense TTG3 gene sequence or hair pin TTG3 construct can be used as a selectable marker. Transformation of a plant line with vector constructs such as P_{35S}-HP-TTG3, P_{35S}-antisense-TTG3 or P_{35S}-antisense-TTG3-B will result in down-regulation of the TTG3 gene expression and manifestation of at least a ttg3 phenotype. Selection based on a ttg3 mutant phenotype allows for selection of transformed plants.

### Example 26

### Tissue Specific ttg3 Expression Results in Phenotype Dissection

Inhibition of TTG3 in the seed only can yield a yellow seed phenotype with leaves having normal trichome development. Conversely, inhibition of TTG3 in leaf tissue only may inhibit trichome development and yet allow for a normal seed phenotype. Numerous promoters are available that have expression profiles which one may select as appropriate. Alternatively, one may which to over-express a TTG3 gene in a tissue or developmentally specific manner. Selection of appropriate promoter elements can be done to achieve this goal.

1. An isolated nucleic acid sequence comprising the nucleotide sequence of SEQ ID NO:1, SEQ ID NO:2, SEQ ID NO:6, SEQ ID NO:7 or a fragment thereof.

2. The nucleic acid of claim 1, wherein said fragment is at least 120 nucleotides in length.

3. The nucleic acid of claim 1, wherein said fragment is at least 200 nucleotides in length.

4. The nucleic acid of claim 1, wherein said fragment is at least 300 nucleotides in length.

5. The nucleic acid of claim 1, wherein said fragment is at least 500 nucleotides in length.

6. The nucleic acid of claim 1, wherein said fragment is at 750 nucleotides in length.

7. The isolated nucleic acid of claim 1, wherein said nucleic acid is RNA.

8. A vector comprising the nucleic acid of claim 1.

9. A cell comprising the vector of claim 8.

10. An isolated nucleic acid sequence comprising the sequence of SEQ ID NO:5 or SEQ ID NO:8.

11. An isolated nucleic acid construct comprising, a first nucleic acid sequence comprising SEQ ID NO:5 or SEQ ID NO:8 or fragment thereof operably linked to a second nucleic acid sequence, wherein expression of said second nucleic acid sequence is regulated by said first nucleic acid sequence.

12. An isolated nucleic acid construct comprising, a first nucleic acid sequence comprising SEQ ID NO:5 or SEQ ID NO:8 or fragment thereof operably linked to a second nucleic acid sequence encoding a heterologous gene, wherein said heterologous gene encodes a protein of interest.

13. A vector comprising the nucleic acid construct of claim 11.

14. A cell comprising the vector of claim 13.

15. A vector comprising the nucleic acid construct of claim 12.

16. A cell comprising the vector of claim 15.

17. A plant comprising the cell of claim 14.

18. A plant comprising the cell of claim 16.

19. A plant having altered expression of a TTG3 gene, or a gene homolog of TTG3 due to a non-naturally occuring mutation of said TTG3 gene; wherein, the plant displays at least a phenotype selected from the group of altered seed oil content, altered seed fiber content, altered trichome production, altered trichome structure, transparent testa, altered anthocyanin production, altered proanthocyaninidin production and altered flavanol production compared to a wild type plant.

20. The seed produced by the plant of claim 19, wherein said seed produces a plant that has an altered phenotype selected from the group consisting of altered seed oil content, altered seed fiber content, altered trichome production, altered trichome structure, transparent testa, altered anthocyanin production, altered proanthocyaninidin production and altered flavanol production compared to a wild type plant.

21. A method of producing a transgenic plant, comprising introducing into a plant cell a nucleic acid comprising at least a portion of a TTG3 nucleic acid sequence to generate a transgenic cell; and regenerating a transgenic plant from said transgenic cell.

22. The method of claim 21, wherein said plant has an altered phenotype selected from the group consisting of altered seed oil content, altered seed fiber content, altered trichome production, altered trichome structure, transparent testa, altered anthocyanin production, altered proanthocyaninidin production and altered flavanol production compared to a wild type plant.

23. The method claim 21, wherein said TTG3 nucleic acid is in the sense or the anti-sense orientation

24. The method of claim 21, wherein said TTG3nucleic acid comprises SEQ ID NO: 1, SEQ ID NO: 2, SEQ ID NO: 6 or SEQ ID NO: 7 or a fragment thereof.

25. The method of claim 21, wherein said TTG3 nucleic acid comprises at least 20 consecutive nucleotides complementary to SEQ ID NO: 1, SEQ ID NO: 2, SEQ ID NO: 6 or SEQ ID NO: 7.

26. The method of claim 21, wherein said promoter is selected from the group consisting of a constitutive promoter, an inducible promoter, an ABA inducible promoter, tissue specific promoters or a guard cell-specific promoter.

27. The transgenic plant produced by the method of claim 21.

28. The seed produced by the transgenic plant of claim 27, wherein said seed produces a plant that has an altered phenotype selected from the group consisting of altered seed oil content, altered seed fiber content, altered trichome production, altered trichome structure, transparent testa, altered anthocyanin production, altered proanthocyaninidin production and altered flavanol production compared to a wild type plant.

29. A method of producing a transgenic plant having decreased TTG3 expression comprising:
(a) transforming a plant, tissue culture or plant cell with a nucleic acid construct comprising a promoter operably linked to a nucleic acid sequence that is anti-sense to at least a portion of a TTG3 nucleic acid sequence; and
(b) growing the plant or regenerating a plant from the tissue culture or plant cell.

30. The transgenic plant produced by the method of claim 29.

31. The seed produced by the transgenic plant of claim 30, wherein said seed the produces a plant that displays at least a phenotype selected from the group of increased seed oil content, decreased seed fiber content, reduced trichome production, altered trichome structure, transparent testa, reduced anthocyanin production reduced proanthocyaninidin production and reduced flavanol production compared to a wild type plant.

32. A method of producing a transgenic plant having decreased TTG3 expression comprising:
(a) transforming a plant, tissue culture or plant cell with a nucleic acid construct comprising a promoter operably linked to a portion of a nucleic acid sequence comprising a TTG3 nucleic acid sequence in a sense orientation; and
(b) growing the plant or regenerating a plant from the tissue culture or plant cell.

33. The transgenic plant produced by the method of claim 32.

34. The seed produced by the transgenic plant of claim 33, wherein said seed produces a plant that displays at least a phenotype selected from the group of increased seed oil content, decreased seed fiber content, reduced trichome production, altered trichome structure, transparent testa, reduced anthocyanin production reduced proanthocyaninidin production and reduced flavanol production compared to a wild type plant.

35. A method of producing a transgenic plant having increased TTG3 expression comprising:
(a) transforming a plant, tissue culture or plant cell with nucleic acid construct comprising a promoter operably linked to a portion of a nucleic acid sequence comprising a TTG3 nucleic acid sequence in a sense orientation; and
(b) growing the plant or regenerating a plant from the tissue culture or plant cell.

36. The transgenic plant produced by the method of claim 35.

37. The seed produced by the transgenic plant of claim 36, wherein said seed produces a plant that displays at a phenotype of decreased seed oil content, or increased seed fiber content compared to a wild type plant.

38. A method of detecting transformation of a ttg3 deficient plant with a DNA construct, the method comprising:
(a) transforming a plant, tissue culture or plant cell with a construct comprising a wild-type TTG3 nucleic acid sequence operably linked to a promoter that is functional in said plant cell in a sense orientation;
(b) growing the plant or regenerating a plant from the tissue culture or plant cell; and
(c) detecting the reversion of at least a ttg3 phenotype to a wild-type phenotype, wherein the appearance of a wild-type phenotype is indicative of transformation.

39. The method of claim 38, wherein the DNA construct comprises a second nucleic acid sequence operably linked to a promoter that is functional in a plant cell.

40. A method of detecting transformation of a plant with a DNA construct, the method comprising:
(a) transforming a plant, tissue culture or plant cell with a nucleic acid construct comprising an antisense TTG3 nucleic acid sequence operably linked to a promoter that is functional in said plant cell;
(b) growing the plant or regenerating a plant from the tissue culture or plant cell; and
(c) detecting at least a ttg3 phenotype, wherein the appearance of a ttg3 phenotype is indicative of transformation.

41. The method of claim 40, wherein the DNA construct comprises a second nucleic acid sequence operably linked to a promoter that is functional in a plant cell.


SEQUENCE LISTING (80 sequences)

SEQ ID NO: 1 - DNA, 1007 bp, Arabidopsis thaliana
aaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctctctcaaaattttcagagagagagattgacatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagataagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaa

SEQ ID NO: 2 - DNA, 1164 bp, Arabidopsis thaliana
aaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctctctcaaaattttcagagagagagattgacatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagataagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaacacgtgtctgagatatattagcttgatccaatcttcttctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 3 - DNA, 366 bp, Arabidopsis thaliana
atggtgagtggcctatattcattattctttttagaatcatacaagagtttttgcttaaaatgtttatcgagtcacggctattatattttttctgttactattagagagaatcaaaccaaaattttcgacgacggtaaattgatgtcgtcggtgatggttgaattgccgtccggtgtagtatccggctttcgtctgacatatattggcttccacagcagtacatgttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaa

SEQ ID NO: 4 - protein, 121 aa, Arabidopsis thaliana
MVSGLYSLFFLESYKSFCLKCLSSHGYYIFSVTIRENQTKIFDDGKLMSSVMVELPSGVVSGFRLTYIGFHSSTCCWKNSDEANLRVSFDLVSVSGRIDGFTAKDDRWKTFVSSGYMCIRC

SEQ ID NO: 5 - DNA, 1102 bp, Arabidopsis thaliana
gcgaatacgtaaacgtatgacctggtttgttcggtttgcgagttaaagtcaatttgaagaaaaagagtaatttaagataagctaatatgaaatctgatctaatgtggcatttcaatattgtaatatcttgcttgataaagagaaaacaaataatgcgaatgcggcatctctctctcttgtctccacgtcaacgattttgctagatttgattccaatcccttttccagcttacctaccttttccctcaaaaaattgtgttaaatatggcttcatctcttaagaaatactttccattttggaagtaatttcctaatttcttagcaaggaaaaaaacctaaaaaggaaatgactataacattttataatgtaaaaggattaaaaaaaagggtagaatatcataattatcatttttacattgttaagaaacttatttatgtatccatggttacttttgtttattttttataattaaaacaaaaaaagtaattccttttaatttatcattaaattcgaaagtataaaaagcgttggaccacaatccattctgagaaaaaaccccatcctatgaaatttaactcataaaagtgtcaaaatgttcttgaaaattcaaaatttatcatgaataaaactttaaatatttctactcactattcttattttttagtatatatatctatatattatatttagaatccttcacaaaaataattgttgcataatactcattggttttacacgttttctgggctcaagttttgttccaatttttttgatatatgaaaattctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatg

SEQ ID NO: 6 - DNA, 881 bp, Arabidopsis thaliana
atgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctctctcaaaattttcagagagagagattgacatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagataagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaa

SEQ ID NO: 7 - DNA, 915 bp, Arabidopsis thaliana
attcttaaagacaaaattaaaatgactctggccctctctctcttctctctctcaaaattttcagagagagaatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagatcagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaacacgtgtctgagatatattagcttgatccaatcttcttctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 8 - DNA, 1215 bp, Arabidopsis thaliana
gcgaatacgtaaacgtatgacctggtttgttcggtttgcgagttaaagtcaatttgaagaaaaagagtaatttaagataagctaatatgaaatctgatctaatgtggcatttcaatattgtaatatcttgcttgataaagagaaaacaaataatgcgaatgcggcatctctctctcttgtctccacgtcaacgattttgctagatttgattccaatcccttttccagcttacctaccttttccctcaaaaaattgtgttaaatatggcttcatctcttaagaaatactttccattttggaagtaatttcctaatttcttagcaaggaaaaaaacctaaaaaggaaatgactataacattttataatgtaaaaggattaaaaaaaagggtagaatatcataattatcatttttacattgttaagaaacttatttatgtatccatggttacttttgtttattttttataattaaaacaaaaaaagtaattccttttaatttatcattaaattcgaaagtataaaaagcgttggaccacaatccattctgagaaaaaaccccatcctatgaaatttaactcataaaagtgtcaaaatgttcttgaaaattcaaaatttatcatgaataaaactttaaatatttctactcactattcttattttttagtatatatatctatatattatatttagaatccttcacaaaaataattgttgcataatactcattggttttacacgttttctgggctcaagttttgttccaatttttttgatatatgaaaattctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcatttt

SEQ ID NO: 9 - DNA, 512 bp, Arabidopsis thaliana
gaagcagcagaagccttgatcatcttcctttgtctcaacctgaaactcttttttttctttcattgtttgttctcttttcactgtggatgtagataattgtttttaatgaaatgaagaaatattgatttgccttttgacataattttgttaataatcttgattacaaattttagtcagtgtttgatgcatagttgcatactgcagagttgagtttggatatggccacgtcagcattatctcgttaccaaaacgtaaggtccaaactcagataatacaaacgaagcagttctttgtcactctatcatcaacatatgaaccacaccaaaaaagaacaaaatcgtagataatgatcatgcaaaaccgaccgttggatcttactttcgatttcaaaccacataaatcttagtgactgagctaaaaaactgaaattttttaaaaggcaagacctcctctgtttccatattctcaccacagaagaactcttgaggctttctcttttctctaccatggcg

SEQ ID NO: 10 - DNA, 522 bp, Arabidopsis thaliana
attgcttaaggccagattctgtgaaacatggacaagaacagagcaagttatgttgaattgactcgtgtaattcgtgaaacagaacatagcaagtccaagttgtgttaaaaactgcagagaatttgacagattggtggaagtaaaaagcattcttttgcaactcattttaagatcggcaaagaaaaaattgaagtaacagaaccttactgtaacactattcgttactctaaagctgtgttatattgtttagagacagaaataatcaaactcttgtgataatttggtagatgataacaaatcagaactctgaaggtcaatcttttttgattcttaggtgaagacaagttggttatttcaaagatcacgtgcttaccttctaaaacagccttattgatctactgttgtacctaatgagcaaggactatttgcaaatctttttacttcttatatagaagtctcaagacgataaactcataacaactaaatctctatctctgtaatttcaaaagtacaatcatggac

SEQ ID NO: 11 - DNA, 510 bp, Arabidopsis thaliana
aagcttaaattgacattatatatgaaagacaataaataagatacaacgttttatataaacaatatggattatatacaatagttaaaaaatccttagatgtctgttaaacacagcgttttggtgagttaactcgtatggtataagtttaaagcattccagtaactatattggtatcaaaatctacattcatattaattcaaataattttttacgtttaccaaaataaatccatataaacatgcgattgatcttgggcaagagaggtgttaaaatagtatcttggtatgactattagttgaaatttgactaatgcattcaagaatgtatatttattgtaagaaatgctcacgtatcttatttgataaagaatgctcacgtaccttaataaatacgtgtgcatagagtttgtgtgtatatatagatggttatgatcagggaagaagaacacaacacaactcacctcaaacagacaatttaattccaaaagataaaaacaatcaaaagagatct

SEQ ID NO: 12 - DNA, 343 bp, Arabidopsis thaliana
cttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaatgtagattaaaaacatacacg

SEQ ID NO: 13 - DNA, 1265 bp, Arabidopsis thaliana
gcgaatacgtaaacgtatgacctggtttgttcggtttgcgagttaaagtcaatttgaagaaaaagagtaatttaagataagctaatatgaaatctgatctaatgtggcatttcaatattgtaatatcttgcttgataaagagaaaacaaataatgcgaatgcggcatctctctctcttgtctccacgtcaacgattttgctagatttgattccaatcccttttccagcttacctaccttttccctcaaaaaattgtgttaaatatggcttcatctcttaagaaatactttccattttggaagtaatttcctaatttcttagcaaggaaaaaaacctaaaaaggaaatgactataacattttataatgtaaaaggattaaaaaaaagggtagaatatcataattatcatttttacattgttaagaaacttatttatgtatccatggttacttttgtttattttttataattaaaacaaaaaaagtaattccttttaatttatcattaaattcgaaagtataaaaagcgttggaccacaatccattctgagaaaaaaccccatcctatgaaatttaactcataaaagtgtcaaaatgttcttgaaaattcaaaatttatcatgaataaaactttaaatatttctactcactattcttattttttagtatatatatctatatattatatttagaatccttcacaaaaataattgttgcataatactcattggttttacacgttttctgggctcaagttttgttccaatttttttgatatatgaaaattctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctct

SEQ ID NO: 14 - DNA, 1003 bp, Arabidopsis thaliana
tatggcttcatctcttaagaaatactttccattttggaagtaatttcctaatttcttagcaaggaaaaaaacctaaaaaggaaatgactataacattttataatgtaaaaggattaaaaaaaagggtagaatatcataattatcatttttacattgttaagaaacttatttatgtatccatggttacttttgtttattttttataattaaaacaaaaaaagtaattccttttaatttatcattaaattcgaaagtataaaaagcgttggaccacaatccattctgagaaaaaaccccatcctatgaaatttaactcataaaagtgtcaaaatgttcttgaaaattcaaaatttatcatgaataaaactttaaatatttctactcactattcttattttttagtatatatatctatatattatatttagaatccttcacaaaaataattgttgcataatactcattggttttacacgttttctgggctcaagttttgttccaatttttttgatatatgaaaattctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctct

SEQ ID NO: 15 - DNA, 707 bp, Arabidopsis thaliana
catcctatgaaatttaactcataaaagtgtcaaaatgttcttgaaaattcaaaatttatcatgaataaaactttaaatatttctactcactattcttattttttagtatatatatctatatattatatttagaatccttcacaaaaataattgttgcataatactcattggttttacacgttttctgggctcaagttttgttccaatttttttgatatatgaaaattctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctct

SEQ ID NO: 16 - DNA, 483 bp, Arabidopsis thaliana
attctaaggtctataaacatattggatgcaatgaaaaggtctaaataaacaaaatcacaatctatcttcacaacaatttcttagaaattcaaacttcacataagttgtttgaatccttttttaattaaaaggatatattttaatatgagttaactaaaataattagacacttaaaccaaattaaaagtaataaattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctct

SEQ ID NO: 17 - DNA, 291 bp, Arabidopsis thaliana
aattctttacttaccaatccggtggagacacgtgagcccctatggcaataaacttataaccttcttccgccactgcctatcacaacacctattagctttgatgtgtggtggacagtggacttgtaatgatggtgagtggcctatattcattattctttttagaatcatacaagagtttttgcttaaaatgtttatcgagtcacggctattatattttttctgttactattgtattcattttattcttaaagacaaaattaaaatgactctggccctctctctcttctctct

SEQ ID NO: 18 - DNA, 44 bp, Artificial Sequence
  Description of Artificial Sequence chemically synthesized adapter
ctaatacgactcactatagggctcgagcggccgcccgggcaggt

SEQ ID NO: 19 - DNA, 8 bp, Artificial Sequence
  Description of Artificial Sequence chemically synthesized adapter
acctgccc

SEQ ID NO: 20 - DNA, 27 bp, Artificial Sequence
  Description of Artificial Sequence chemically synthesized primer
ggatcctaatacgactcactatagggc

SEQ ID NO: 21 - DNA, 20 bp, Artificial Sequence
  Description of Artificial Sequence chemically synthesized primer
agctggcgtaatagcgaaga

SEQ ID NO: 22 - DNA, 18 bp, Artificial Sequence
  Description of Artificial Sequence chemically synthesized primer
ctatagggctcgagcggc

SEQ ID NO: 23 - DNA, 20 bp, Artificial Sequence
  Description of Artificial Sequence chemically synthesized primer
cgttggagtccacgttcttt

SEQ ID NO: 24 - DNA, 32 bp, Artificial Sequence
  Description of Artificial Sequence chemically synthesized primer
aaatctagaatgatggtgagtggcctatattc

SEQ ID NO: 25 - DNA, 41 bp, Artificial Sequence
  Description of Artificial Sequence chemically synthesized primer
aaagagctcttagcatcgaatacacatgtaacctgaggaga

SEQ ID NO: 26 - DNA, 41 bp, Artificial Sequence
  Description of Artificial Sequence chemically synthesized primer
aaatctagagcgaatacgtaaacgtatgacctggtttgttc

SEQ ID NO: 27 - DNA, 29 bp, Artificial Sequence
  Description of Artificial Sequence chemically synthesized primer
aaagagctcatggataattcagctccaga

SEQ ID NO: 28 - DNA, 32 bp, Artificial Sequence
  Description of Artificial Sequence chemically synthesized primer
aaagagctcactctaaggagctgcattttgtt

SEQ ID NO: 29 - DNA, 20 bp, Artificial Sequence
  Description of Artificial Sequence chemically synthesized primer
gagattcttcacatccaggg

SEQ ID NO: 30 - DNA, 20 bp, Artificial Sequence
  Description of Artificial Sequence chemically synthesized primer
catctcgtccattccctcac

SEQ ID NO: 31 - DNA, 39 bp, Artificial Sequence
  Description of Artificial Sequence chemically synthesized primer
aaacccgggtacaagtccactgtccaccacacatcaaag

SEQ ID NO: 32 - DNA, 32 bp, Artificial Sequence
  Description of Artificial Sequence chemically synthesized primer
aaaggatccatgatggtgagtggcctatattc

SEQ ID NO: 33 - DNA, 41 bp, Artificial Sequence
  Description of Artificial Sequence chemically synthesized primer
aaatctagattagcatcgaatacacatgtaacctgaggaga

SEQ ID NO: 34 - DNA, 40 bp, Artificial Sequence
  Description of Artificial Sequence chemically synthesized primer
aaatctagagattcttaaagacaaaattaaaatgactctg

SEQ ID NO: 35 - DNA, 39 bp, Artificial Sequence
  Description of Artificial Sequence chemically synthesized primer
aaaggatcccacaagtgacgattttattaagatgaacat

SEQ ID NO: 36 - DNA, 23 bp, Artificial Sequence
  Description of Artificial Sequence chemically synthesized primer
gccgacagtggtcccaaagatgg

SEQ ID NO: 37 - DNA, 18 bp, Artificial Sequence
  Description of Artificial Sequence chemically synthesized primer
gcaagaccggcaacagga

SEQ ID NO: 38 - DNA, 34 bp, Artificial Sequence
  Description of Artificial Sequence chemically synthesized primer
aaaggatccacagcagtacatggtcggcccttgt

SEQ ID NO: 39 - DNA, 35 bp, Artificial Sequence
  Description of Artificial Sequence chemically synthesized primer
aaatctagatgaacataacatacaacgaacgagca

SEQ ID NO: 40 - DNA, 34 bp, Artificial Sequence
  Description of Artificial Sequence chemically synthesized primer
aaagagctcacagcagtacatggtcggcccttgt

SEQ ID NO: 41 - DNA, 35 bp, Artificial Sequence
  Description of Artificial Sequence chemically synthesized primer
aaagagctctgaacataacatacaacgaacgagca

SEQ ID NO: 42 - DNA, 37 bp, Artificial Sequence
  Description of Artificial Sequence chemically synthesized primer
aaaggatccgaccgttgattgtctttagatgatcaga

SEQ ID NO: 43 - DNA, 37 bp, Artificial Sequence
  Description of Artificial Sequence chemically synthesized primer
aaatctagaccatctatcatccttcgccgtgaagcca

SEQ ID NO: 44 - DNA, 37 bp, Artificial Sequence
  Description of Artificial Sequence chemically synthesized primer
aaagagctcgaccgttgattgtctttagatgatcaga

SEQ ID NO: 45 - DNA, 37 bp, Artificial Sequence
  Description of Artificial Sequence chemically synthesized primer
aaagagctcccatctatcatccttcgccgtgaagcca

SEQ ID NO: 46 - DNA, 32 bp, Artificial Sequence
  Description of Artificial Sequence chemically synthesized primer
aaagagctcatgatggtgagtggcctatattc

SEQ ID NO: 47 - DNA, 29 bp, Artificial Sequence
  Description of Artificial Sequence chemically synthesized primer
aaaaagcttattgcttaaggccagattct

SEQ ID NO: 48 - DNA, 33 bp, Artificial Sequence
  Description of Artificial Sequence chemically synthesized primer
aaatctagacttgatgagacttgatgagaatag

SEQ ID NO: 49 - DNA, 33 bp, Artificial Sequence
  Description of Artificial Sequence chemically synthesized primer
aaatctagacttttgattgtttttatcttttgg

SEQ ID NO: 50 - DNA, 35 bp, Artificial Sequence
  Description of Artificial Sequence chemically synthesized primer
aaaaagcttaaattgacattatatatgaaagacaa

SEQ ID NO: 51 - DNA, 35 bp, Artificial Sequence
  Description of Artificial Sequence chemically synthesized primer
aaaagcttttaaaccaacttagtaaacgttttttt

SEQ ID NO: 52 - DNA, 39 bp, Artificial Sequence
  Description of Artificial Sequence chemically synthesized primer
aaatctagacgtgtatgtttttaatctacattgtattga

SEQ ID NO: 53 - DNA, 29 bp, Artificial Sequence
  Description of Artificial Sequence chemically synthesized primer
aaaaagcttgaagcagcagaagccttgat

SEQ ID NO: 54 - DNA, 30 bp, Artificial Sequence
  Description of Artificial Sequence chemically synthesized primer
aaaggatcccgccatggtagagaaaagaga

SEQ ID NO: 55 - DNA, 20 bp, Artificial Sequence
  Description of Artificial Sequence chemically synthesized primer
gaagcagcagaagccttgat

SEQ ID NO: 56 - DNA, 40 bp, Artificial Sequence
  Description of Artificial Sequence chemically synthesized primer
aaatctagatatggcttcatctcttaagaaatactttcca

SEQ ID NO: 57 - DNA, 41 bp, Artificial Sequence
  Description of Artificial Sequence chemically synthesized primer
aaatctagacatcctatgaaatttaactcataaaagtgtca

SEQ ID NO: 58 - DNA, 39 bp, Artificial Sequence
  Description of Artificial Sequence chemically synthesized primer
aaatctagaattctaaggtctataaacatattggatgca

SEQ ID NO: 59 - DNA, 38 bp, Artificial Sequence
  Description of Artificial Sequence chemically synthesized primer
aaatctagaaattctttacttaccaatccggtggagac

SEQ ID NO: 60 - DNA, 38 bp, Artificial Sequence
  Description of Artificial Sequence chemically synthesized primer
aatcccgggagagagaagagagagagggccagagtcat

SEQ ID NO: 61 - DNA, 39 bp, Artificial Sequence
  Description of Artificial Sequence chemically synthesized primer
attcccgggattcttaaagacaaaattaaaatgactctg

SEQ ID NO: 62 - DNA, 39 bp, Artificial Sequence; identical to SEQ ID NO: 35
  Description of Artificial Sequence chemically synthesized primer

SEQ ID NO: 63 - DNA, 32 bp, Artificial Sequence
  Description of Artificial Sequence chemically synthesized primer
attcccgggtctagcatcatcggctctttcag

SEQ ID NO: 64 - DNA, 36 bp, Artificial Sequence
  Description of Artificial Sequence chemically synthesized primer
attcccgggtatcctttgagtttaaggtttacggat

SEQ ID NO: 65 - DNA, 37 bp, Artificial Sequence
  Description of Artificial Sequence chemically synthesized primer
attcccgggtccttacttctgttttgtagttgttgga

SEQ ID NO: 66 - DNA, 35 bp, Artificial Sequence
  Description of Artificial Sequence chemically synthesized primer
attcccgggtctctaaatgtaatgttgggccagtt

SEQ ID NO: 67 - DNA, 30 bp, Artificial Sequence
  Description of Artificial Sequence chemically synthesized primer
aaaggatccatctatcatccttcgccgtga

SEQ ID NO: 68 - DNA, 37 bp, Artificial Sequence
  Description of Artificial Sequence chemically synthesized primer
aaaggatccagcttgaatataagagatacagctaaag

SEQ ID NO: 69 - DNA, 36 bp, Artificial Sequence
  Description of Artificial Sequence chemically synthesized primer
aaaggatcctctaaagacaatcaacggtcgaaacgt

SEQ ID NO: 70 - DNA, 36 bp, Artificial Sequence
  Description of Artificial Sequence chemically synthesized primer
aaaggatcctcaaccatcaccgacgacatcaattta

SEQ ID NO: 71 - DNA, 915 bp, Artificial Sequence; identical to SEQ ID NO: 7
  Description of Artificial Sequence chemically synthesized expression construct

SEQ ID NO: 72 - DNA, 718 bp, Artificial Sequence
  Description of Artificial Sequence chemically synthesized deletion construct
tctagcatcatcggctctttcagcggtgatggctagctcacggtgttaatccggcgtagttcggttttcttttccttttggtttatatcacggttacgtttcgaccgttgattgtctttagatcagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaacacgtgtctgagatatattagcttgatccaatcttcttctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 73 - DNA, 517 bp, Artificial Sequence
  Description of Artificial Sequence chemically synthesized deletion construct
tatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaacacgtgtctgagatatattagcttgatccaatcttcttctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 74 - DNA, 319 bp, Artificial Sequence
  Description of Artificial Sequence chemically synthesized deletion construct
tccttacttctgttttgtagttgttggaaaaattcagatgaagctaatcttcgagtttcattcgacttagtttcagtttccggcagaatcgatggcttcacggcgaaggatgatagatggaaaactttcgtctcctcaggttacatgtgtattcgatgctaacacgtgtctgagatatattagcttgatccaatcttcttctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 75 - DNA, 120 bp, Artificial Sequence
  Description of Artificial Sequence chemically synthesized deletion construct
tctctaaatgtaatgttgggccagttggacttaaaatagtctctgtaaaccgttttatgttgttgggcttttgctcgttcgttgtatgttatgttcatcttaataaaatcgtcacttgtg

SEQ ID NO: 76 - DNA, 717 bp, Artificial Sequence
  Description of Artificial Sequence chemically synthesized deletion construct
attcttaaagacaaaattaaaatgactctggccctctctctcttctctctctcaaaattttcagagagagaatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagatcagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagcttcgattgggtctaagtaacagaggtggttaatcgaagatcttttttctcttttgagattaatagatccgtattagatccttacttctgttttgtagttgttggaaaaattcagatgaagctaatcttcgagtttcattcgacttagtttcagtttccggcagaatcgatggcttcacggcgaaggatgatagatgga

SEQ ID NO: 77 - DNA, 520 bp, Artificial Sequence
  Description of Artificial Sequence chemically synthesized deletion construct
attcttaaagacaaaattaaaatgactctggccctctctctcttctctctctcaaaattttcagagagagaatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttagatcagaattcattgggtatgtgtgttggtgatggcgttggttttcagtccaagcttgcttaattagatctaattccaacctatcctttgagtttaaggtttacggataataattaggcagtttgcgtttttgagatgaagattcaaagctctttcctcagcttattatgattgcctttagctgtatctcttatattcaagct

SEQ ID NO: 78 - DNA, 319 bp, Artificial Sequence
  Description of Artificial Sequence chemically synthesized deletion construct
attcttaaagacaaaattaaaatgactctggccctctctctcttctctctctcaaaattttcagagagagaatcaaaccaaaattttcgacgacggtaaattgatgtcgtcggtgatggttgaattgccgtccggtgtagtatccggctttcgtctgacatatattggcttccacagcagtacatggtcggcccttgtctagcatcatcggctctttcagcggtgatggctagctcacggtgttaatccggcgtagttcggttttcttttccttttggtttatatcacggttacgtttcgaccgttgattgtctttaga

SEQ ID NO: 79 - DNA, 123 bp, Artificial Sequence
  Description of Artificial Sequence chemically synthesized deletion construct
attcttaaagacaaaattaaaatgactctggccctctctctcttctctctctcaaaattttcagagagagaatcaaaccaaaattttcgacgacggtaaattgatgtcgtcggtgatggttga

SEQ ID NO: 80 - DNA, 1164 bp, Arabidopsis thaliana
cacaagtgacgattttattaagatgaacataacatacaacgaacgagcaaaagcccaacaacataaaacggtttacagagactattttaagtccaactggcccaacattacatttagagaagaagattggatcaagctaatatatctcagacacgtgttagcatcgaatacacatgtaacctgaggagacgaaagttttccatctatcatccttcgccgtgaagccatcgattctgccggaaactgaaactaagtcgaatgaaactcgaagattagcttcatctgaatttttccaacaactacaaaacagaagtaaggatctaatacggatctattaatctcaaaagagaaaaaagatcttcgattaaccacctctgttacttagacccaatcgaagcttgaatataagagatacagctaaaggcaatcataataagctgaggaaagagctttgaatcttcatctcaaaaacgcaaactgcctaattattatccgtaaaccttaaactcaaaggataggttggaattagatctaattaagcaagcttggactgaaaaccaacgccatcaccaacacacatacccaatgaattcttatctaaagacaatcaacggtcgaaacgtaaccgtgatataaaccaaaaggaaaagaaaaccgaactacgccggattaacaccgtgagctagccatcaccgctgaaagagccgatgatgctagacaagggccgaccatgtactgctgtggaagccaatatatgtcagacgaaagccggatactacaccggacggcaattcaaccatcaccgacgacatcaatttaccgtcgtcgaaaattttggtttgatgtcaatctctctctctgaaaattttgagagagagaagagagagagggccagagtcattttaattttgtctttaagaataaaatgaatacaatagtaacagaaaaaatataatagccgtgactcgataaacattttaagcaaaaactcttgtatgattctaaaaagaataatgaatataggccactcaccatcattacaagtccactgtccaccacacatcaaagctaataggtgttgtgataggcagtggcggaagaaggttataagtttattgccataggggctcacgtgtctccaccggattggtaagtaaagaattt